(19) United States
(12) Patent Application Publication
(10) Pub. No.: US 2011/0165183 A1
(43) Pub. Date: Jul. 7, 2011

(54) PIPERIDINE DERIVATIVES AS JAK3 INHIBITORS

(75) Inventors: Yarlagadda S. Babu, Birmingham, AL (US); Pooran Chand, Birmingham, AL (US); Pravin L. Kotian, Birmingham, AL (US); V. Satish Kumar, Birmingham, AL (US)

(73) Assignee: BioCryst Pharmaceuticals, Inc., Birmingham, AL (US)

(21) Appl. No.: 13/057,100

(22) PCT Filed: Jul. 31, 2009
(86) PCT No.: PCT/US2009/052449

§ 371 Date: Feb. 1, 2011

Related U.S. Application Data
(60) Provisional application No. 61/085,705, filed on Aug. 1, 2008.
(60) Provisional application No. 61/098,562, filed on Sep. 19, 2008.

(51) Int. Cl. A61K 39/00; C07D 491/048; A61K 31/519; C07D 487/04; A61K 31/53; C07D 513/04

(52) U.S. Cl. 424/184.1; 544/278; 514/260.1; 544/183; 514/243; 544/184; 514/259.3; 544/281; 514/259.31; 544/263; 544/280; 514/265.1; 514/258.1; 544/255

(57) ABSTRACT

The invention provides a compound of formula (I): wherein W is a bicyclic heteroaromatic group; or a salt thereof. The compounds and salts thereof have beneficial therapeutic properties (e.g. immunosuppressant properties).

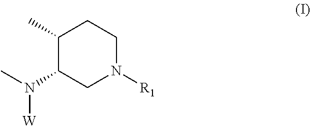

### CROSS-REFERENCE TO RELATED APPLICATIONS

This patent application claims the benefit of priority of U.S. application Ser. No. 61/085,705, filed Aug. 1, 2008 and of U.S. application Ser. No. 61/098,562, filed Sep. 19, 2008, which applications are herein incorporated by reference.

### BACKGROUND OF THE INVENTION

As discussed by Elizabeth Kudlacz et al. (American Journal of Transplantation, 2004, 4, 51-57), Janus kinase 3 (JAK3) is a cytoplasmic protein tyrosine kinase associated with the common gamma chain (γc), which is an integral component of various cytokine receptors.

While effective in the prevention of transplant rejection, commonly used immunosuppressants, such as calcineurin inhibitors, possess a number of significant dose-limiting toxicities, thereby prompting a search for agents with novel mechanisms of action. The inhibition of JAK3 represents an attractive strategy for immunosuppression based upon its limited tissue distribution, lack of constitutive activation and the evidence for its role in immune cell function. JAK3 is a viable target for immunosuppression and transplant rejection. Jak-3 specific inhibitors may also be useful for treatment of hematologic and other malignancies that involve pathologic Jak activation.

Currently, there is a need for compounds, compositions and methods that are useful for treating diseases and conditions associated with pathologic Jak activation.

### SUMMARY OF THE INVENTION

In one embodiment, the invention provides a compound of the invention which is a compound of formula I:

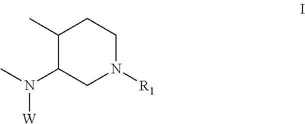

wherein:

R₁ is H, alkyl, cycloalkyl, (cycloalkyl)alkyl, heterocycle, heteroaryl, aryl, wherein any alkyl, cycloalkyl, (cycloalkyl)alkyl, or heterocycle of R₁ may be optionally substituted with one or more (e.g. 1, 2, 3, 4 or 5) Rₐ, and wherein any heteroaryl or aryl, of R₁ may be optionally substituted with one or more (e.g. 1, 2, 3, 4 or 5) R_{c}; or R₁ is —C(R_{g})(Rₕ)—C(Rₖ)(Rₘ)—CN;

each Rₐ group is independently selected from halogen, aryl, heteroaryl, heterocycle, R_{b}, OH, CN, OR_{b}, —O-aryl, —O-heterocycle, —O-heteroaryl, —OC(O)R_{b}, —OC(O)NHR_{b}, oxo, SH, SR_{b}, —S-aryl, —S-heteroaryl, —S(O)R_{b}, —S(O)aryl, —S(O)heteroaryl, —S(O)₂OH, —S(O)₂R_{b}, —S(O)₂aryl, —S(O)₂heteroaryl, —S(O)₂NH₂, —S(O)₂NHR_{b}, —S(O)₂NR_{b}R_{b}, —NH₂, —NHR_{b}, —NR_{b}R_{b}, —NHCOR_{b}, —NHCOaryl —NHCOheteroaryl, —NHCO₂R_{b}, —NHCONH₂, —NHCONHR_{b}, —NHS(O)₂R_{b}, —NHS(O)₂aryl, —NHS(O)₂NH₂, NO₂, ═NOR_{b}, CHO, —C(O)R_{b}, —C(O)OH, —C(O)OR_{b}, —C(O)NH₂, —C(O)NHR_{b}, —C(O)NR_{b}R_{b}, —C(O)heterocycle, —C(O)heteroaryl and —C(O)C(O)R_{b} and wherein any aryl, heteroaryl, or heterocycle of Rₐ may be optionally substituted with one or more (e.g. 1, 2, 3, 4 or 5) R_{c} groups;

each R_{b} is independently lower alkyl or lower cycloalkyl wherein lower alkyl or lower cycloalkyl may be optionally substituted with one or more (e.g. 1, 2 or 3) groups selected from halogen, CN, OH, —O-lower alkyl, —NH-lower alkyl, —C(O)NH-lower alkyl, —C(O)N(lower alkyl)₂, heterocycle and heteroaryl which heterocycle may be substituted with one or more (e.g. 1, 2 or 3) lower alkyl;

each R_{c} is independently halogen, aryl, R_{d}, OH, CN, OR_{d}, —Oaryl, —OC(O)R_{d}, —OC(O)NHR_{d}, SH, SR_{d}, —S-aryl, —S-heteroaryl, —S(O)R_{d}, —S(O)aryl, —S(O)heteroaryl, —S(O)₂OH, —S(O)₂R_{d}, —S(O)₂aryl, —S(O)₂heteroaryl, —S(O)₂NHR_{d}, —S(O)₂NR_{d}R_{d}, —NH₂, —NHR_{d}, —NR_{d}R_{d}, —NHCOR_{d}, —NHCOaryl, —NHCOheteroaryl, —NHCO₂R_{d}, —NHCONH₂, —NHCONHR_{d}, —NHS(O)₂R_{d}, —NHS(O)₂aryl, —NHS(O)₂NH₂, NO₂, CHO, —C(O)R_{d}, —C(O)OH, —C(O)OR_{d}, —C(O)NH₂, —C(O)NHR_{d}, —C(O)NR_{d}R_{d}, —C(O)cyclic amino, —C(O)C(O)R_{d}, heterocycle or heteroaryl wherein any aryl may be optionally substituted with one or more (e.g. 1, 2, 3, 4 or 5) Rₑ groups;

each R_{d} is independently lower alkyl or lower cycloalkyl wherein lower alkyl or lower cycloalkyl may be optionally substituted with one or more (e.g. 1, 2 or 3) groups selected from halogen, CN, OH, —O-lower alkyl, —NH-lower alkyl, —C(O)NH-lower alkyl, —C(O)N(lower alkyl)₂, heterocycle and heteroaryl which heterocycle may be substituted with one or more (e.g. 1, 2 or 3) lower alkyl;

each Rₑ is independently halogen, aryl, R_{f}, OH, CN, OR_{f}, —Oaryl, —OC(O)R_{f}, —OC(O)NHR_{f}, oxo, SH, SR_{f}, —S-aryl, —S-heteroaryl, —S(O)R_{f}, —S(O)aryl, —S(O)heteroaryl, —S(O)₂OH, —S(O)₂R_{f}, —S(O)₂aryl, —S(O)₂heteroaryl, —S(O)₂NHR_{f}, —S(O)₂NR_{f}R_{f}, —NH₂, —NHR_{f}, —NR_{f}R_{f}, —NHCOR_{f}, —NHCOaryl, —NHCOheteroaryl, —NHCO₂R_{f}, —NHCONH₂, —NHCONHR_{f}, —NHS(O)₂R_{f}, —NHS(O)₂aryl, —NHS(O)₂NH₂, NO₂, CHO, —C(O)R_{f}, —C(O)OH, —C(O)OR_{f}, —C(O)NH₂, —C(O)NHR_{f}, —C(O)NR_{f}R_{d}, —C(O)cyclic amino, —C(O)C(O)R_{d}, heterocycle or heteroaryl;

each R_{f} is independently lower alkyl or lower cycloalkyl wherein lower alkyl or lower cycloalkyl may be optionally substituted with one or more (e.g. 1, 2 or 3) groups selected from halogen, CN, OH, —O-lower alkyl, —NH-lower alkyl, —C(O)NH-lower alkyl, —C(O)N(lower alkyl)₂, heterocycle and heteroaryl which heterocycle may be substituted with one or more (e.g. 1, 2 or 3) lower alkyl;

R_{g} and Rₕ taken together are —CH₂—O—CH₂—;

Rₖ and Rₘ are each H, or taken together with the carbon to which they are attached form a C₃-C₆ spiro-carbocyclic ring; and

W is selected from:

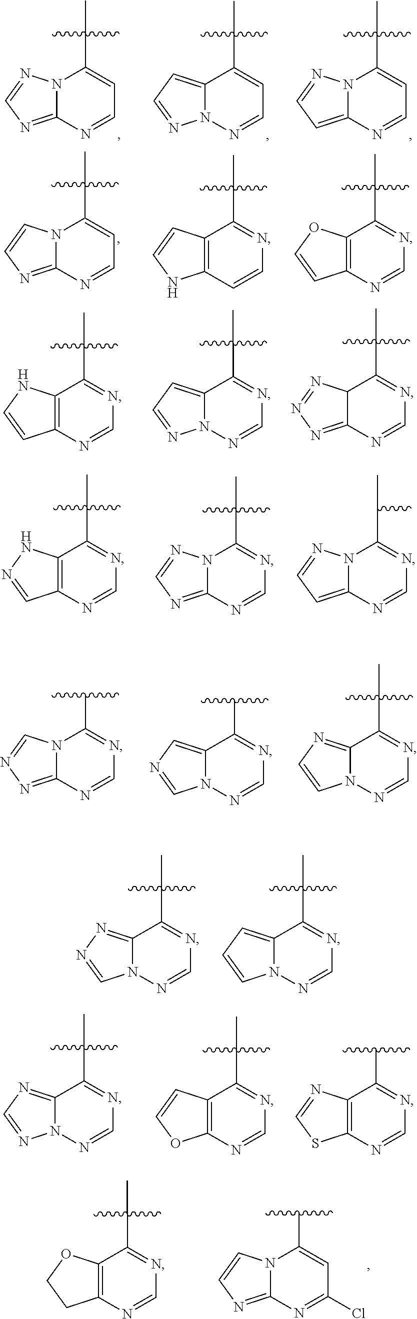

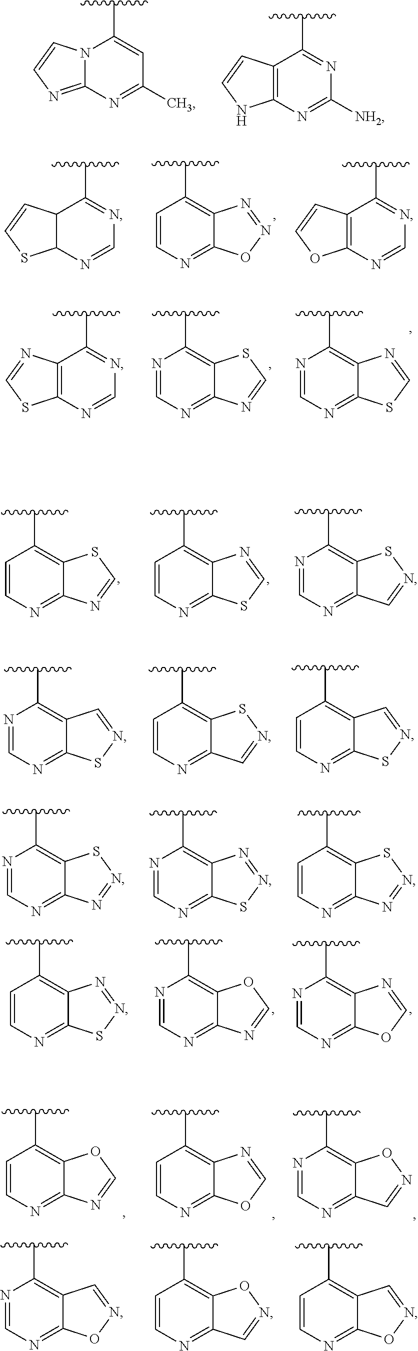

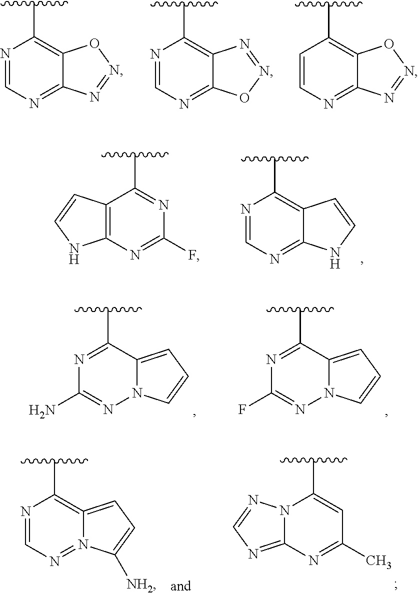

or a salt thereof.

In one embodiment, the invention provides a pharmaceutical composition comprising a compound of formula I, or a pharmaceutically acceptable salt thereof, in combination with a pharmaceutically acceptable diluent or carrier.

In one embodiment, the invention provides method for treating a disease or condition associated with pathologic Jak activation in a mammal, comprising administering a compound of formula I, or a pharmaceutically acceptable salt thereof, to the mammal.

In one embodiment, the invention provides a compound of formula I or a pharmaceutically acceptable salt thereof for use in the prophylactic or therapeutic treatment of a disease or condition associated with pathologic Jak activation (e.g., cancer).

In one embodiment, the invention provides a compound of formula I or a pharmaceutically acceptable salt thereof for use in medical therapy (e.g. for use in treating a disease or condition associated with pathologic Jak activation), as well as the use of a compound of formula I or a pharmaceutically acceptable salt thereof for the manufacture of a medicament useful for the treatment of a disease or condition associated with pathologic Jak activation in a mammal, such as a human.

In one embodiment, the invention provides processes and intermediates disclosed herein (e.g. those illustrated in Schemes 1-7 and in the Examples below) that are useful for preparing compounds of formula I or salts thereof.

### DETAILED DESCRIPTION

The term “alkyl” as used herein refers to alkyl groups having from 1 to 10 carbon atoms which are straight or branched monovalent groups.

The term “lower alkyl” as used herein refers to alkyl groups having from 1 to 6 carbon atoms which are straight or branched monovalent groups. This term is exemplified by groups such as methyl, ethyl, n-propyl, iso-propyl, n-butyl, t-butyl, isobutyl, n-pentyl, neopentyl, and n-hexyl, and the like.

The term “halogen” as used herein refers to fluoro, chloro, bromo and iodo.

The term “cycloalkyl” as used herein refers to a saturated or partially unsaturated cyclic hydrocarbon ring systems, such as those containing 1 to 3 rings and 3 to 8 carbons per ring wherein multiple ring cycloalkyls can have fused and Spiro bonds to one another but not bridging bonds. Therefore, cycloalkyl does not include bridged cyclic hydrocarbons as defined below. Exemplary groups include but are not limited to cyclopropyl, cyclobutyl, cyclopentyl, cyclohexyl, cycloheptyl, cyclooctyl, cyclobutenyl, cyclohexenyl, cyclooctadienyl, decahydronaphthalene and spiro[4.5]decane.

The term “lower cycloalkyl” as used herein refers to a cycloalkyl containing 1 ring and 3-6 carbon atoms. Exemplary groups include cyclopropyl, cyclobutyl, cyclopentyl and cyclohexyl.

The term “aryl” as used herein refers to a monovalent aromatic cyclic group of from 6 to 14 carbon atoms having a single ring (e.g. phenyl) or multiple condensed rings (e.g. naphthyl or anthryl) wherein the condensed rings may be aromatic, saturated or partially saturated provided that at least one of the condensed rings is aromatic. Exemplary aryls include, but are not limited to, phenyl, indanyl naphthyl, 1,2-dihydronaphthyl and 1,2,3,4-tetrahydronaphthyl.

The term “heteroaryl” as used herein refers to a group of from 1 to 10 carbon atoms and 1 to 4 heteroatoms selected from the group consisting of oxygen, nitrogen and sulfur in the ring. The sulfur and nitrogen heteroatoms atoms may also be present in their oxidized forms. Such heteroaryl groups can have a single aromatic ring with at least one heteroatom (e.g. pyridyl, pyrimidinyl or furyl) or multiple condensed rings (e.g. indolizinyl or benzothienyl) wherein all of the condensed rings may or may not be aromatic and/or contain a heteroatom provided that at least one of the condensed rings is aromatic with at least one heteroatom. Exemplary heteroaryl groups include, but are not limited to pyridyl, pyrrolyl, pyrazinyl, pyrimidinyl, pyridazinyl, pyrazolyl, thienyl, indolyl, thiophenyl, imidazolyl, oxazolyl, thiazolyl, furyl, oxadiazolyl, thiadiazolyl, quinolyl, isoquinolyl, benzothiazolyl, benzoxazolyl, indazolyl, indolyl, quinoxalyl, quinazolyl, 5,6,7,8-tetrahydroisoquinoline and the like.

The term “heterocycle” or “heterocyclic” or “heterocycloalkyl” refers to a group of from 1 to 10 carbon atoms and 1 to 4 heteroatoms selected from the group consisting of oxygen, nitrogen and sulfur in the ring. The sulfur and nitrogen heteroatoms atoms may also be present in their oxidized forms. Such heterocycle groups include a single saturated or partially unsaturated ring with at least one heteroatom (e.g. azetidinyl or piperidinyl). Heterocycle groups also include multiple condensed rings wherein the condensed rings may be aryl, cycloalkyl or heterocycle but not heteroaryl provided that at lease one of the condensed rings is a heterocycle (i.e. a saturated or partially unsaturated ring with at least one heteroatom). Heterocycles do not included aza-bridged cyclic hydrocarbons as defined below. Heterocycles may include aziridinyl, azetidinyl, pyrrolizinyl, piperidinyl, homopiperidinyl, morpholinyl, thiomorpholinyl, piperazinyl, tetrahydrofuranyl, tetrahydrothiophenyl, dihydrooxazolyl, tetrahydropyranyl, tetrahydrothiopyranyl, 1,2,3,4-tetrahydroquinolyl, 1,2,3,4-tetrahydroisoquinolyl, benzoxazinyl and dihydrooxazolyl.

The term “cyclic amino” as used herein is a subgroup of heterocycloalkyls and refers to a monovalent 3-membered to 8-membered saturated or partially unsaturated, single, nonaromatic ring which has at least one nitrogen atom, and may have one or more identical or different hetero atoms selected from the group consisting of nitrogen, oxygen, and sulfur wherein the nitrogen or sulfur atoms may be oxidized. Aza-bridged cyclic hydrocarbons are excluded. Cyclic amino includes but is not limited to values such as aziridino, azetidino, pyrrolidino, piperidino, homopiperidino, morpholino, thiomorpholino, and piperazino.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

In cases where compounds are sufficiently basic or acidic, a salt of a compound of formula I can be useful as an intermediate for isolating or purifying a compound of formula I. Additionally, administration of a compound of formula I as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartrate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

A specific compound of formula I is

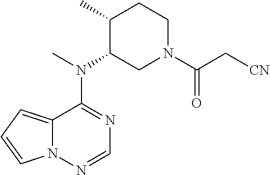

or a salt thereof.

Another specific compound of formula I is

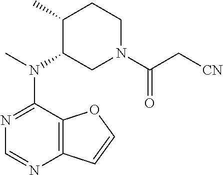

or a salt thereof.

In one embodiment of the invention, the compound of formula I is not:

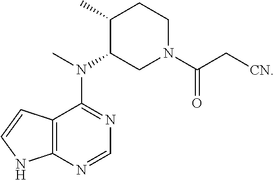

In one specific embodiment the invention provides a compound of formula I which is a compound of formula Ia:

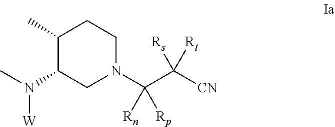

wherein:

Rₙ and Rₚ taken together are oxo (═O) or —CH₂—O—CH₂—;

Rₛ and Rₜ are each H, or taken together with the carbon to which they are attached form a C₃-C₆ spiro-carbocyclic ring; and

W has any of the values defined in claim 1;

or a salt thereof.

In one specific embodiment the invention provides a compound of formula I which is a compound of formula Ib:

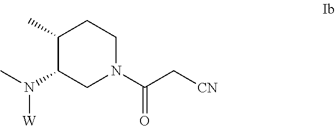

wherein W is selected from:

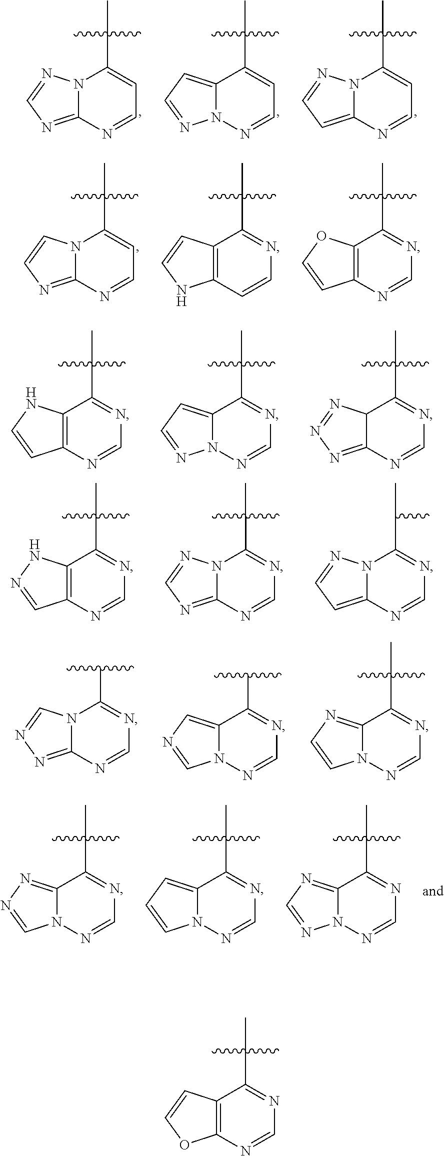

or a salt thereof.

In one specific embodiment of the invention W is selected from:

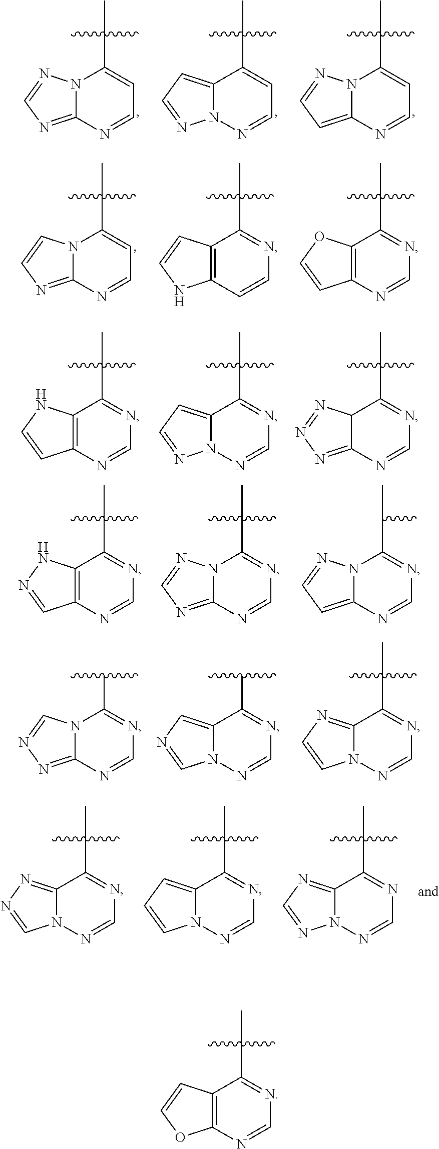

In one specific embodiment of the invention, W is not

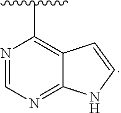

In one specific embodiment of the invention Rₙ and Rₚ taken together are oxo (═O).

In one specific embodiment of the invention Rₙ and Rₚ taken together are —CH₂—O—CH₂—.

In one specific embodiment of the invention Rₛ and Rₜ are each H.

In one specific embodiment of the invention Rₛ and Rₜ taken together with the carbon to which they are attached form a C₃-C₆ spiro-carbocyclic ring.

In one specific embodiment of the invention Rₛ and Rₜ taken together with the carbon to which they are attached form a C₃ spiro-carbocyclic ring.

In one specific embodiment of the invention W is selected from:

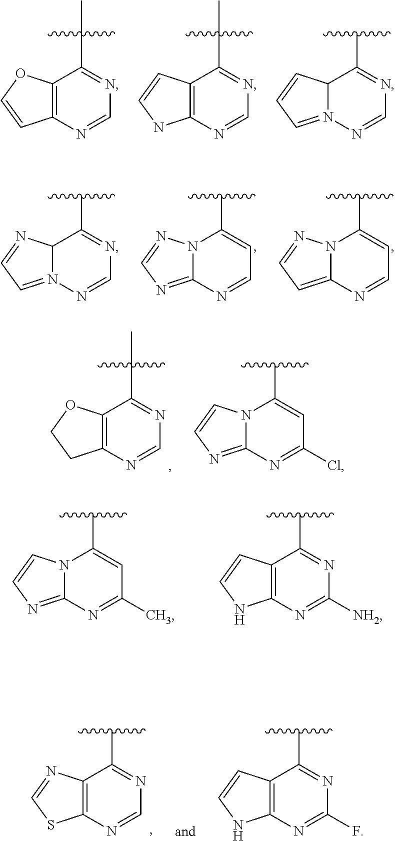

In one specific embodiment of the invention W is selected from:

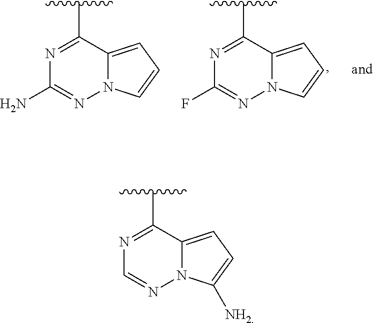

In one specific embodiment the invention provides the compound

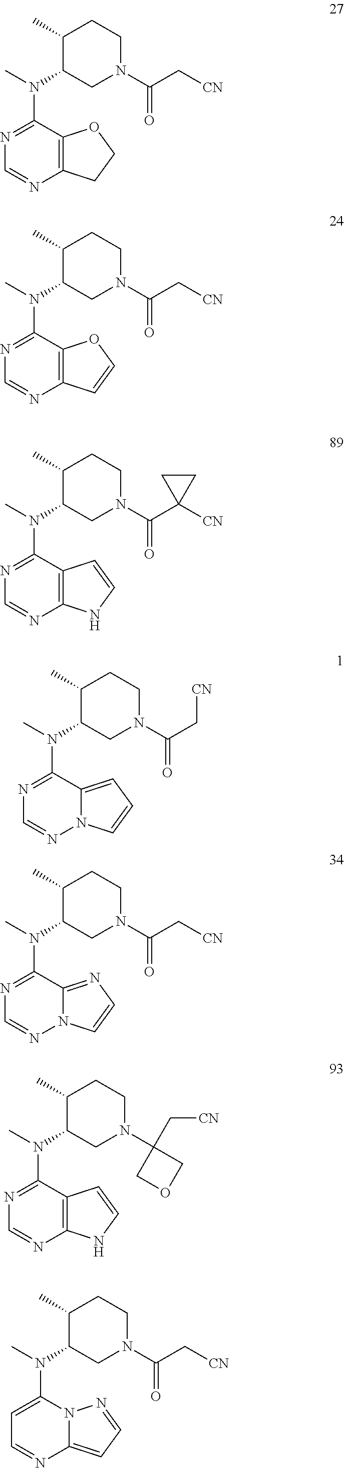

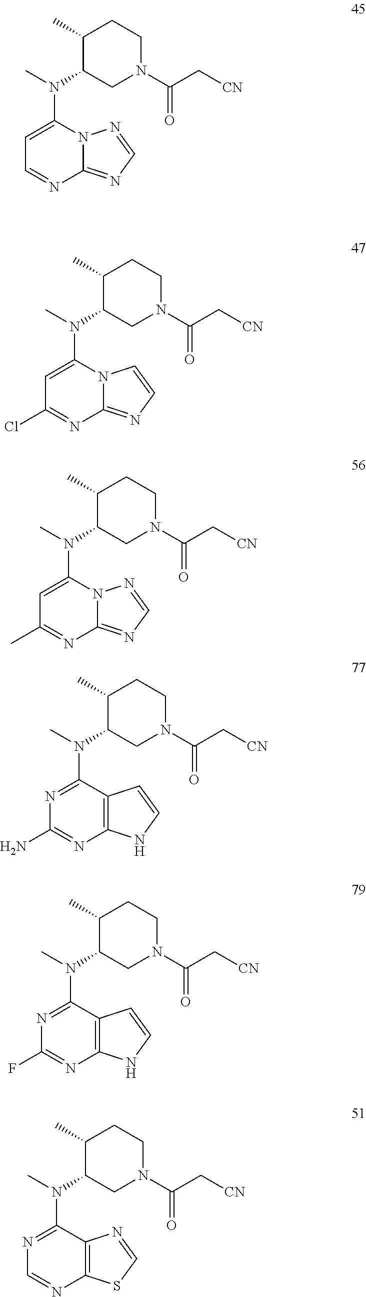

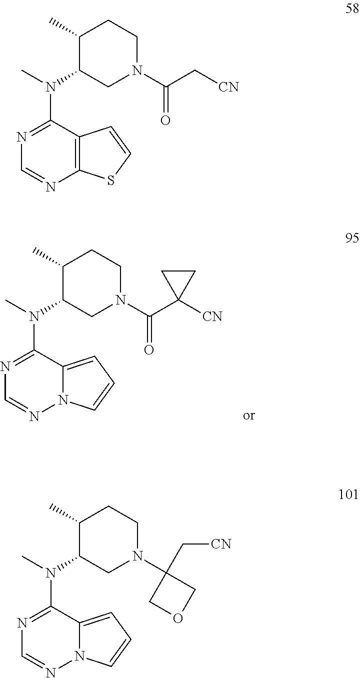

or a salt thereof.

In one specific embodiment the invention provides the compound

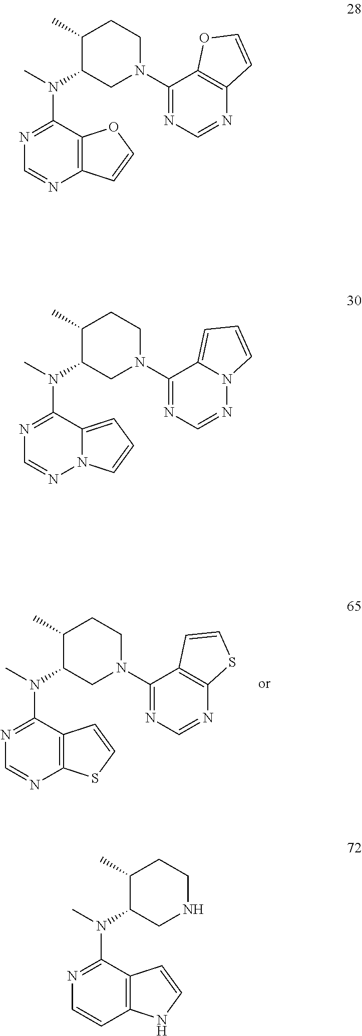

or a salt thereof.

In one specific embodiment the invention provides the compound:

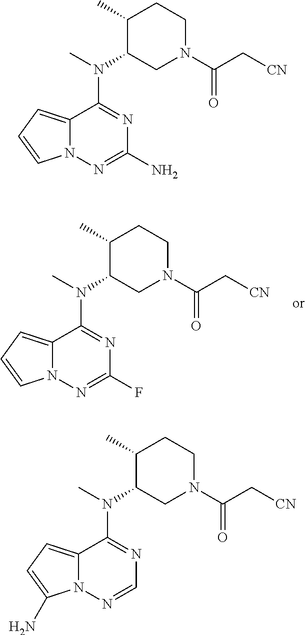

or a salt thereof.

In one specific embodiment of the invention the compound of formula I is a compound of formula Ic:

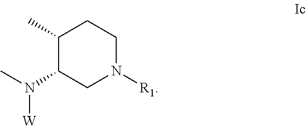

In one specific embodiment of the invention R₁ is alkyl, cycloalkyl, (cycloalkyl)alkyl, heterocycle, heteroaryl, aryl, wherein any alkyl, cycloalkyl, (cycloalkyl)alkyl, or heterocycle of R₁ may be optionally substituted with one or more (e.g. 1, 2, 3, 4 or 5) Rₐ, and wherein any heteroaryl or aryl, of R₁ may be optionally substituted with one or more (e.g. 1, 2, 3, 4 or 5) R_{c}; or R₁ is —C(R_{g})(Rₕ)—C(Rₖ)(Rₘ)—CN.

In one specific embodiment of the invention R₁ is cycloalkyl, (cycloalkyl)alkyl, heterocycle, heteroaryl, aryl, wherein any cycloalkyl, (cycloalkyl)alkyl, or heterocycle of R₁ may be optionally substituted with one or more (e.g. 1, 2, 3, 4 or 5) Rₐ, and wherein any heteroaryl or aryl, of R₁ may be optionally substituted with one or more (e.g. 1, 2, 3, 4 or 5) R_{c}; or R₁ is —C(R_{g}(Rₕ)—C(Rₖ)(Rₘ)—CN.

In one specific embodiment of the invention R₁ is heterocycle, which is optionally substituted with one or more (e.g. 1, 2, 3, 4 or 5) Rₐ.

In one specific embodiment of the invention R₁ is —C(R_{g})(Rₕ)—C(Rₖ)(Rₘ)—CN.

Processes for preparing compounds of formula I are provided as further embodiments of the invention and are illustrated in Schemes 1, 2, and 3.

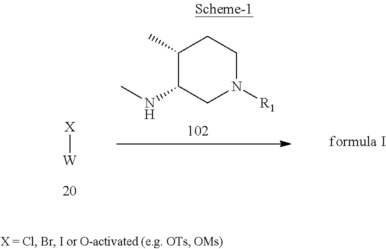

A general method for preparing compounds of formula I is shown in Scheme-2. Reacting a corresponding compound (20) with piperidine 102 (or a salt of 102; e.g. HCl) under conditions suitable to displace the leaving group X to provide the compounds of formula I (22).

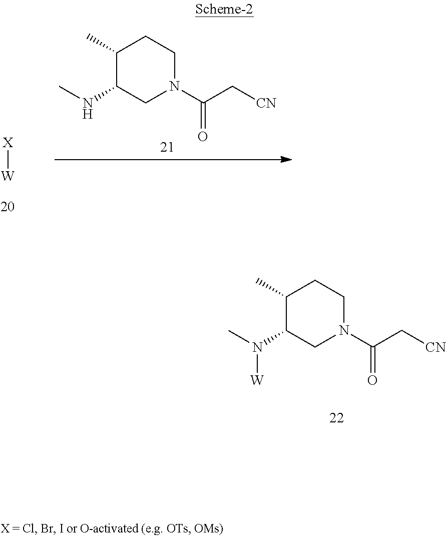

For example, reaction of a compound (20) with piperidine 21 (or a salt of 21; e.g. HCl) under conditions suitable to displace the leaving group X (e.g. Cl, Br, I or activated oxygen) provides the compound of formula I (22).

Additional heteroaryl compounds depicted by structure 20 can be prepared by literature procedures (J. Org. Chem. 1959, 24, 793; J. Med. Chem. 2008, 51, 3649; US2007082901; Justus Liebigs Annalen der Chemie 1962, 657, 141; Nucleosides & Nucleotides 1994, 13(8), 1739; J. Chem. Soc. Chem. Commun. 1993, 840; Liebigs. Ann. Chem. 1993, 367; J. Med. Chem. 1998, 41, 4021; J. Am. Chem. Soc. 1956, 78, 2418; J. Heterocycl. Chem. 1974, 199; Tetrahedron, 1970, 26, 3357; Ger. Offen. Patent DE 2349504, 1973; J. Am Chem. Soc. 2006, 128, 15372; and Tetrahedron Lett. 2007, 48, 5261). When the compound contains a hydroxyl group the hydroxyl group can be converted to a chloro, bromo or iodo or an activated hydroxyl (e.g. OTosyl, OMesyl) according to known literature procedures.

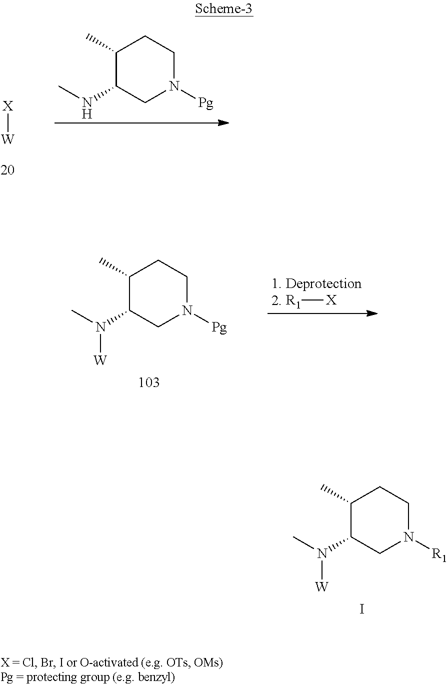

Reaction of a heteroaryl compound (20) with protected piperidine (or a salt thereof) under conditions suitable to displace the leaving group X of the heteroaryl compound provides the protected piperidine intermediate 103, which can be deprotected to provide the corresponding free piperidine 104, which can be allowed to react with a compound of formula R₁—X (wherein X is a suitable leaving group) to provide the compound of formula I.

Processes for preparing intermediate heteroaryl compounds that are useful for preparing compounds of formula I are shown in Schemes 4 and 5.

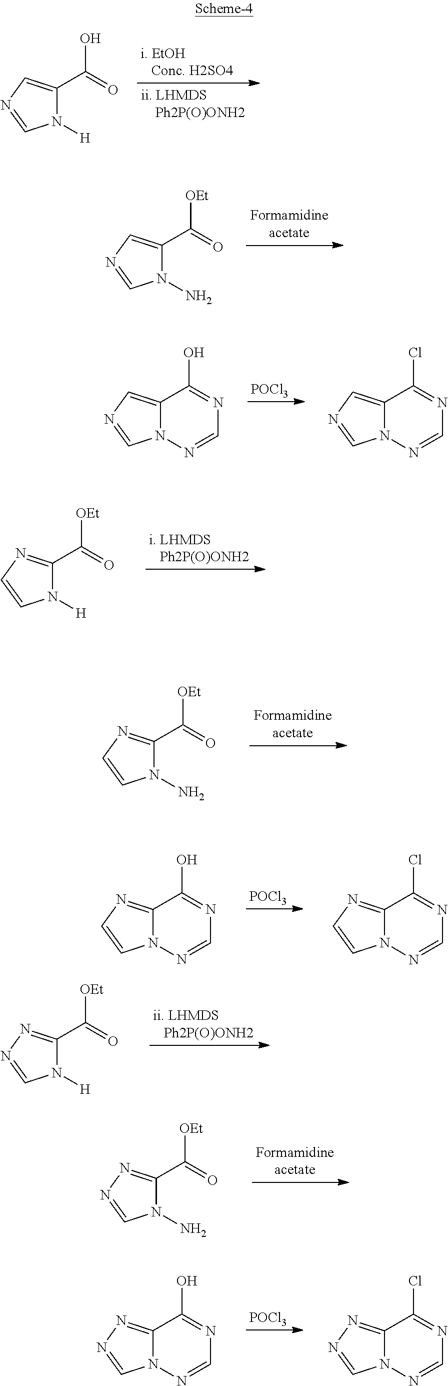

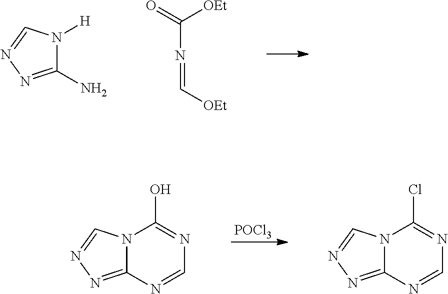

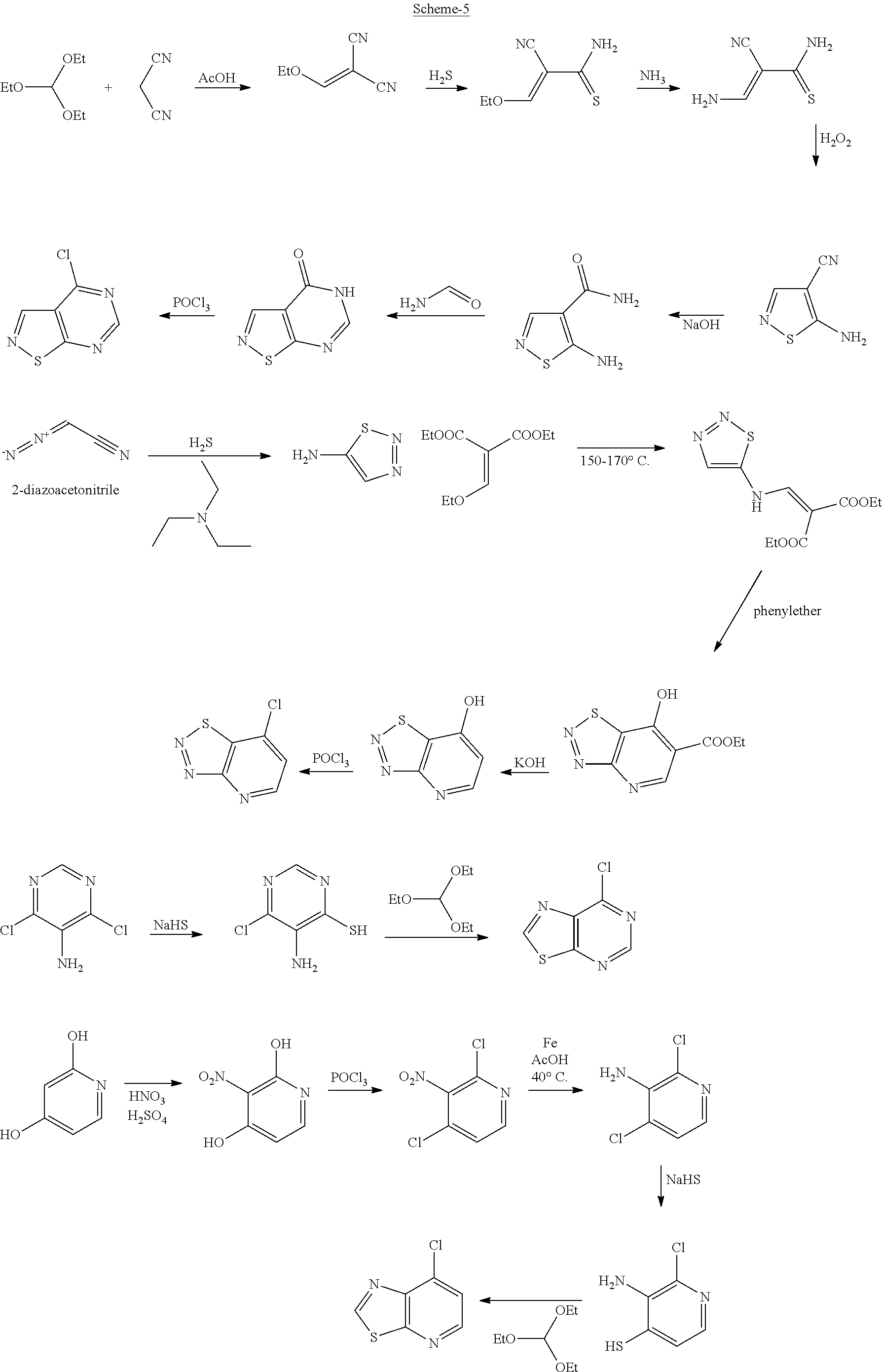

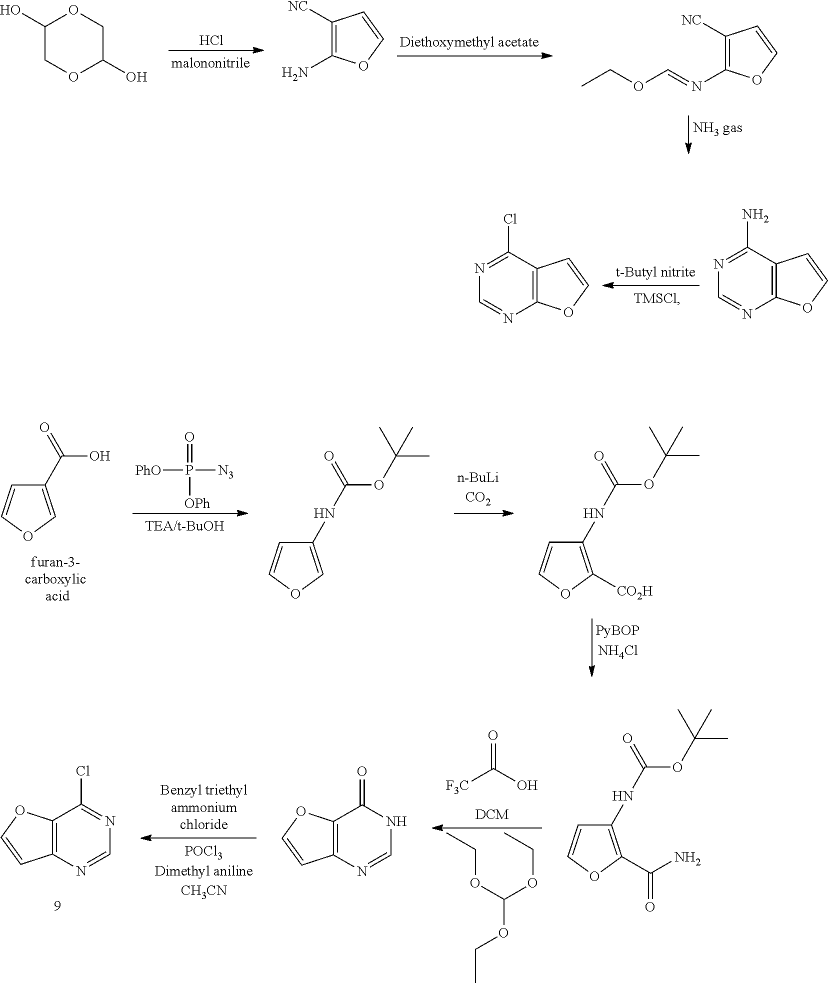

Additional processes for preparing compounds of formula I are provided as further embodiments of the invention and are illustrated in Schemes 6 and 7.

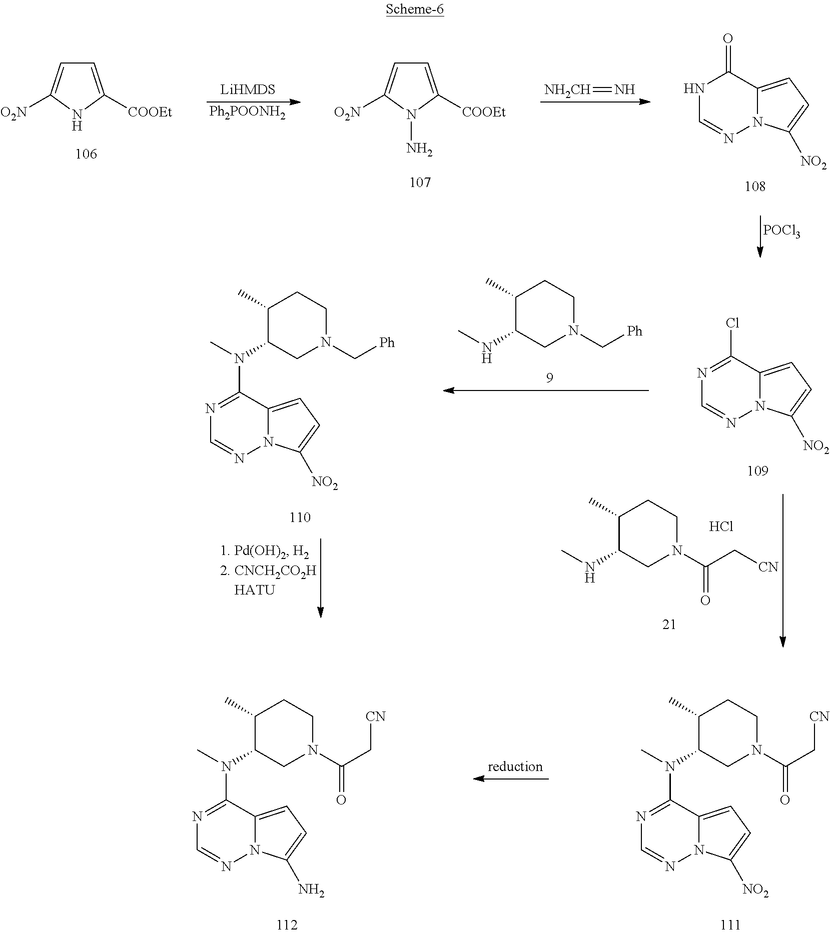

A compound of formula 106 can be prepared according to the procedure reported by Marques et al., Helvetica Chimica Acta, 85(12), 4485-4517 (2002).

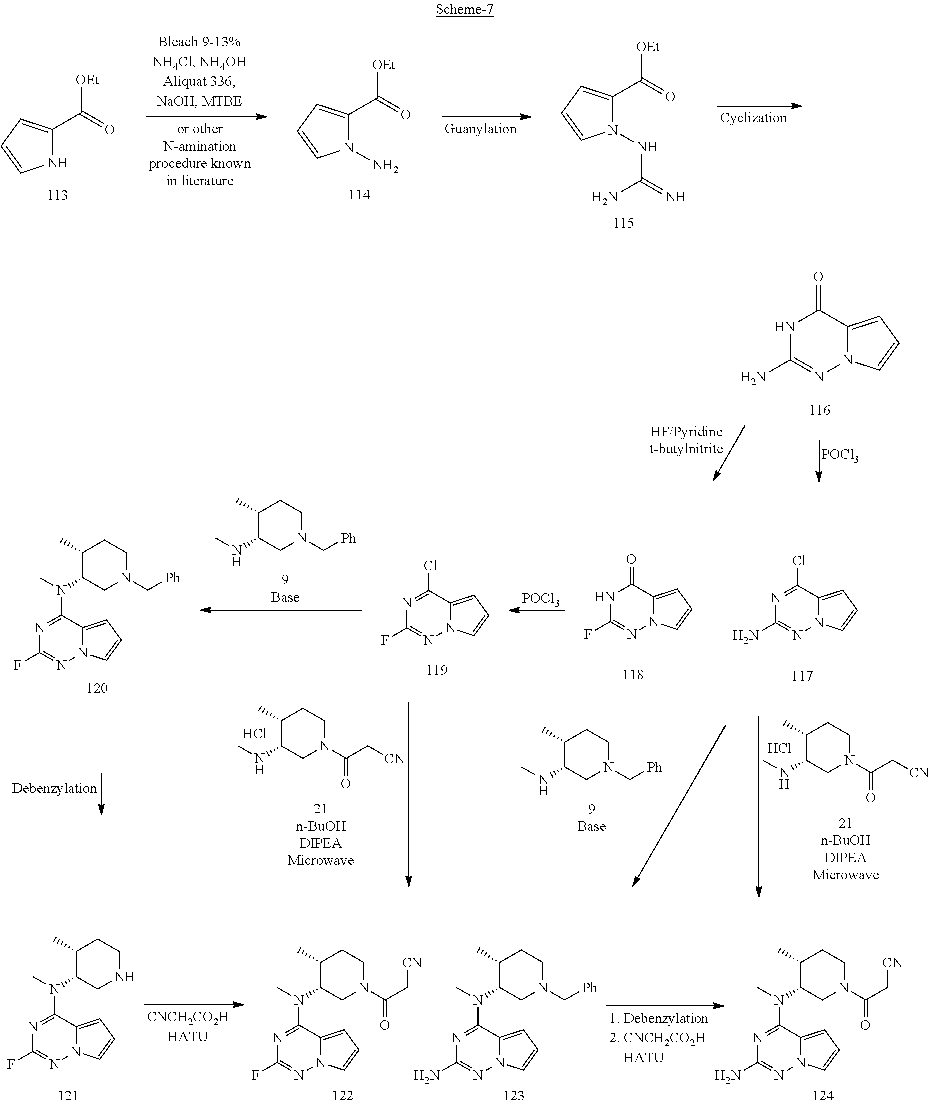

In one embodiment the invention provides a novel process or intermediate compound illustrated in any one of Schemes 1-7.

In another embodiment the invention provides a method for preparing a compound of formula I or a salt thereof comprising:

a. reacting a corresponding compound of formula 20:

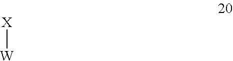

wherein X is a suitable leaving group with a corresponding compound of formula 102:

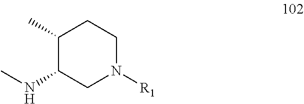

to provide the compound of formula I or the salt thereof; or

b. reacting a corresponding compound of formula 104:

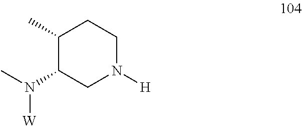

with a corresponding compound of formula R₁—X, wherein X is a suitable leaving group, to provide the compound of formula I.

In one embodiment, the invention provides a method for preparing a salt of a compound of formula I, comprising reacting the compound of formula I with an acid under conditions suitable to provide the salt.

In one embodiment, the invention provides a method for preparing a pharmaceutical composition comprising a compound of formula I, or a pharmaceutically acceptable salt thereof, in combination with a pharmaceutically acceptable diluent or carrier, comprising combining the compound of formula I, or the pharmaceutically acceptable salt thereof, with the pharmaceutically acceptable diluent or carrier to provide the pharmaceutical composition.

The compounds of formula I can be formulated as pharmaceutical compositions and administered to a mammalian host, such as a human patient, in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula Ito the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Compounds of the invention can also be administered in combination with other therapeutic agents, for example, other agents that are useful for immunosuppression. Accordingly, in one embodiment the invention also provides a composition comprising a compound of formula I, or a pharmaceutically acceptable salt thereof, at least one other therapeutic agent, and a pharmaceutically acceptable diluent or carrier. The invention also provides a kit comprising a compound of formula I, or a pharmaceutically acceptable salt thereof, at least one other therapeutic agent, packaging material, and instructions for administering the compound of formula I or the pharmaceutically acceptable salt thereof and the other therapeutic agent or agents to an animal to suppress an immune response in the animal.

The ability of a compound of the invention to bind to Jak-3 may be determined using pharmacological models which are well known to the art, or using Test A described below.

### Test A.

Binding constants (K_{d}'s) were determined against JAK3 (JH1domain-catalytic) kinase. Assays were performed as described in Fabian et al. (2005) Nature Biotechnology, vol. 23, p. 329 and in Karaman et al. (2008) Nature Biotechnology, vol. 26, p. 127. K_{d}s were determined using an 11 point dose response curves which were performed in duplicate. Typically, the observed K_{d} for representative compounds of formula I was less than 10 uM.

The ability of a compound of the invention to provide an immunomodulatory effect can also be determined using pharmacological models which are well known to the art. The ability of a compound of the invention to provide an anti-cancer effect can also be determined using pharmacological models which are well known to the art.

The invention will now be illustrated by the following non-limiting Examples.

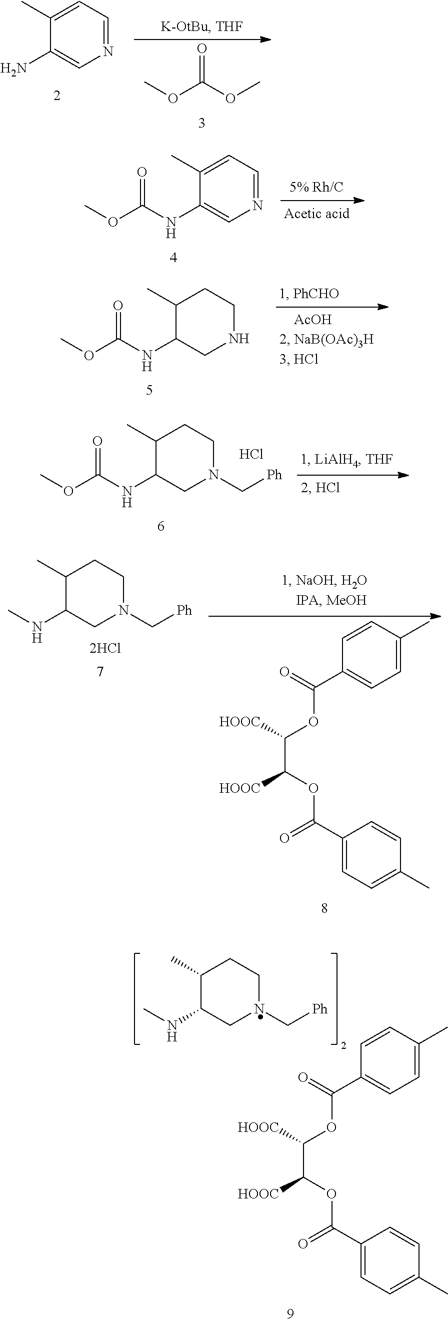

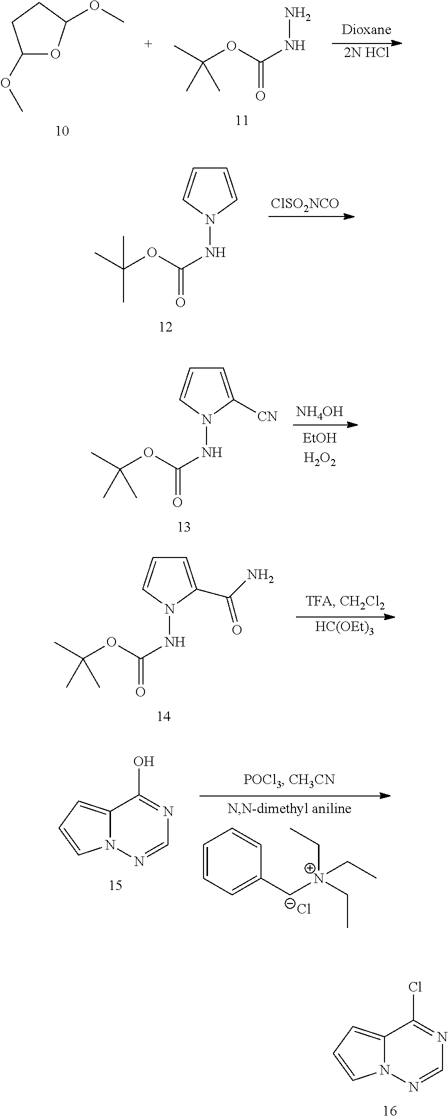

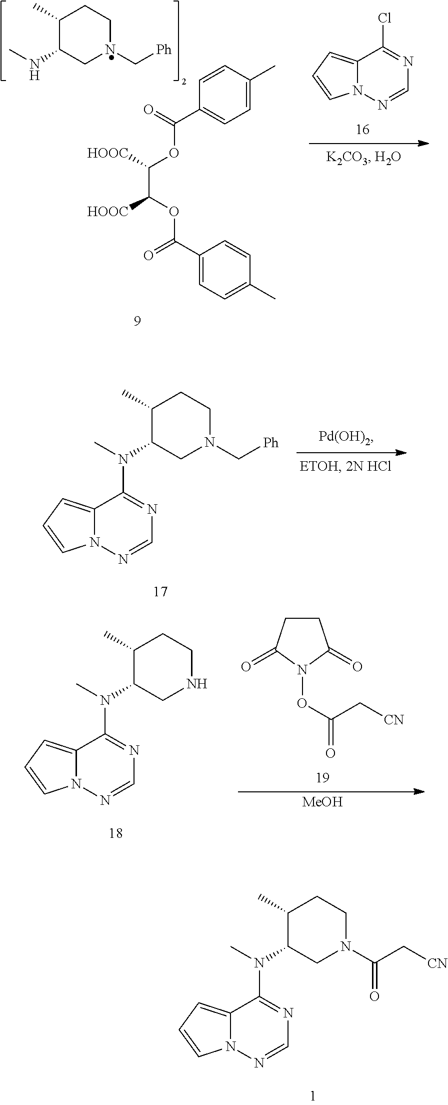

### Example 1

### 3-((3R,4R)-4-methyl-3-(methyl(pyrrolo[1,2-f][1,2,4]triazin-4-yl)amino)piperidin-1-yl)-3-oxopropanenitrile (1)

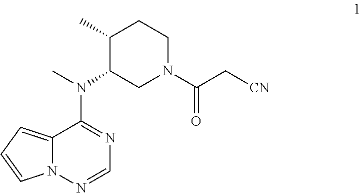

To a stirred suspension of cyano acetic acid (5 g, 58.78 mmol) and N-hydroxysuccinimide (6.76 g, 58.78 mmol) in dichloromethane (100 mL) was added dicychohexyl carbodiimide (12.12 g, 58.78 mmol) at 0° C. The reaction was stirred for 18 hrs at 20° C. The solid separated was filtered and the filtrate was concentrated to afford crude 2,5-dioxopyrrolidin-1-yl 2-cyanoacetate 19 (6.5 g, crude). This was used as such in next step.

To a solution of N-methyl-N-((3R,4R)-4-methylpiperidin-3-yl)pyrrolo[1,2-f][1,2,4]triazin-4-amine 18 (0.1 g, 0.40 mmol) in methanol (5 mL) was added 2,5-dioxopyrrolidin-1-yl 2-cyanoacetate 19 (0.2 g) at 20° C. and stirred at the same temperature for 18 h. Additional 2,5-dioxopyrrolidin-1-yl 2-cyanoacetate 19 (0.2 g) was added and stirred for additional 4 h. The reaction mixture was concentrated in vacuum to remove methanol and the residue obtained was suspended in dichloromethane (20 mL) and filtered. The filtrate was washed with saturated sodium bicarbonate (5 mL), water (15 mL), brine (5 mL), dried, filtered and concentrated in vacuum. The residue obtained was purified by flash chromatography (silica gel, eluting with a mixture of ethyl acetate and methanol (9:1) in hexanes (0 to 50%)) to furnish pure 3-((3R,4R)-4-methyl-3-(methyl(pyrrolo[1,2-f][1,2,4]triazin-4-yl)amino)piperidin-1-yl)-3-oxopropanenitrile (1) (67 mg, 53.6%) as a colorless solid. ¹H NMR (300 MHz, DMSO) δ 7.82 (d, J=4.4, 1H), 7.72 (dd, J=1.5, 2.6, 1H), 6.93 (s, 1H), 6.68 (dd, J=2.7, 4.6, 1H), 4.90 (s, 1H), 4.19-4.02 (m, 2H), 4.00-3.89 (m, 1H), 3.85-3.59 (m, 2H), 3.38 (dd, J=6.8, 18.0, 4H), 2.40 (d, J=6.8, 1H), 1.89-1.65 (m, 1H), 1.65-1.49 (m, 1H), 1.03 (d, J=7.2, 3H); MS (ES⁺): 313.1 (M⁺¹), 335.1 (M+23). HPLC (Zorbax SBC3, 3.0×150 mm, 5 μm, with ZGC SBC3, 2.1×12.5 mm guard cartridge. Mobile phase: 0.1 M ammonium acetate/Acetonitrile) Rt=16.125, (100%).

### Preparation of intermediate compound 18

a. To a stirred solution of potassium tert-butoxide (64.85 g, 577.95 mmol) in tetrahydrofuran (160 mL) was added dimethyl carbonate (36.41 g, 404.56 mmol) by maintaining the temperature below 30° C. To this mixture a solution of 3-amino-4-methylpyridine (25 g, 231.18 mmol) in tetrahydrofuran (100 mL) was added at a rate that maintained the temperature below 30° C. The viscous reaction mixture was diluted with tetrahydrofuran (250 mL) and stirred for 18 h. The reaction was quenched with water (200 mL), the organic layer was separated and washed with brine (100 mL). The aqueous layers were extracted with ethyl acetate (200 mL); washed with water (100 mL) and brine (50 mL). The organic layers were combined dried and concentrated in vacuum. The crude residue obtained was recrystallized from dichloromethane (100 mL) and hexanes (400 mL) to give pure methyl 4-methylpyridin-3-ylcarbamate 4 (34.8 g, 90.5%) as a cream color solid. ¹H NMR (300 MHz, DMSO) δ 9.11 (s, 1H, D2O exchangeable), 8.49 (s, 1H), 8.22 (d, J=4.9, 1H), 7.23 (d, J=4.9, 1H), 3.67 (s, 3H), 2.22 (s, 3H); MS (ES⁺): 167.2 (M⁺¹).189.2 (M+23). Analysis: Calc for C₈H₁₀N₂O₂: C, 57.82; H, 6.06; N, 16.85.

Found: C, 57.70; H, 6.12; N, 16.79.

b. A solution of methyl 4-methylpyridin-3-ylcarbamate 4 (34 g, 204.60 mmol) in acetic acid (400 mL) was degassed for 2 h by bubbling with nitrogen gas. To the solution was added Rhodium on carbon (5%, 50% wet, 5 g) and hydrogenated (150 psi, Hydrogen) at 100° C. (external jacket temperature) for 72 h. The reaction mixture was filtered through celite and concentrated in vacuum. The residue obtained was azeotroped with toluene to furnish crude methyl 4-methylpiperidin-3-ylcarbamate 5 as an acetate salt (57 g). ¹H NMR (300 MHz, DMSO) δ 6.87 (d, J=9.0, 1H, D2O exchangeable), 3.53 (m, 4H, 1H D2O exchangeable), 2.86-2.78 (m, 1H), 2.74 (dd, J=3.4, 13.0, 1H), 2.59 (dd, J=2.7, 12.8, 1H), 2.42 (dt, J=7.9, 21.3, 2H), 1.78-1.60 (m, 1H), 1.34-1.19 (m, 2H), 0.78 (d, J=6.8, 3H); MS (ES⁺): 173.3 (M⁺¹).
c. To a stirred solution of methyl 4-methylpiperidin-3-ylcarbamate 5 (56.17 g, 326.59 mmol) and acetic acid (20 mL) in toluene (500 mL) was added benzaldehyde (51.98 g, 489.89 mmol) at 20° C. The reaction was stirred at the same temperature for 2.5 h. The imine obtained was added to a stirred solution of sodium triacetoxyborohydride (103.82 g, 489.89 mmol) in toluene (300 mL) at 20° C. The reaction was stirred for 18 h at the same temperature and pH was adjusted between 7.0 and 7.5 using aqueous sodium hydroxide (2N). The aqueous layer was separated and extracted with toluene (2×200 mL). The toluene layers were combined, added conc. HCl (70 mL) and heated to 80° C. for about 2 h. The solution was concentrated to dryness and the residue obtained was triturated with toluene. The solid obtained was collected by filtration and dried to afford methyl 1-benzyl-4-methylpiperidin-3-ylcarbamate hydrochloride 6 (36.5 g, 60% from 4) as a colorless crystalline solid.

¹H NMR (300 MHz, CDCl₃) δ 12.31 (s, 1H, D2O exchangeable), 7.62-7.52 (m, 3H), 7.48-7.42 (m, 2H), 4.33-4.14 (m, 2H), 4.06 (d, J=12.9, 1H), 3.65 (s, 3H), 3.52 (d, J=10.8, 1H), 3.31 (d, J=11.5, 1H), 2.91-2.60 (m, 2H), 2.28 (d, J=13.6, 1H), 1.83 (s, 1H), 1.66 (d, J=15.1, 1H), 0.97 (d, J=6.5, 3H); MS (ES⁺): 263.2 (M⁺¹).

d. To a stirred suspension of 1-benzyl-4-methylpiperidin-3-ylcarbamate hydrochloride 6 (35 g, 117 mmol) in tetrahydrofuran (150 mL) was added a solution of lithium aluminum hydride (6.7 g, 175.70 mmol) in tetrahydrofuran (175 mL) at −15° C. The reaction mixture was refluxed for 2 h and cooled to 0° C. The reaction mixture was carefully quenched by adding water and the inorganic salt obtained were filtered off and washed with tetrahydrofuran (100 mL). The filtrate was concentrated in vacuum and to the residue obtained was added isopropanol (500 mL) and added concentrated HCl (50 mL). The mixture was heated at 80° C. for 1.5 h, cooled to room temperature and concentrated in vacuum. The solid obtained was triturated with isopropanol and collected by filtration dried in vacuum to afford cis-1-benzyl-N,4-dimethylpiperidin-3-amine dihydrochloride 7 (29.5 g, 86.4%) as a colorless crystalline solid. ¹H NMR (300 MHz, CH3CN+D2O) δ 7.52 (s, 5H), 4.51-4.23 (m, 2H), 3.62 (d, J=11.4, 2H), 3.18 (d, J=27.3, 3H), 2.70 (s, 3H), 2.51 (s, 1H), 2.03-1.98 (m, 1H), 1.85 (d, J=15.2, 1H), 1.07 (d, J=7.2, 3H); MS (ES⁺): 219.3 (M⁺¹).
e. To a solution of cis-1-benzyl-N,4-dimethylpiperidin-3-amine dihydrochloride 7 (29 g, 99.57 mmol) in water (48.5 mL) was added aqueous sodium hydroxide (2N, 100.56 mL, 201.13 mmol). The slurry was dissolved by adding isopropanol (130.51 mL) and methanol (33.52 mL). To the solution was added Di-p-toluoyl-L-tartaric acid 8 (19.22 g, 49.78 mmol) and heated to reflux until homogenous, cooled to 20° C. and stirred at same temperature for 16 h. The solid separated was collected by filtration and dried in vacuum to afford bis[(1-benzyl-4-methylpiperidin-3-yl)-methylamine] di-p-toluoyl-L-tartarate 9 (16.9 g, 20.6%) as a colorless crystalline solid. ¹H NMR (300 MHz, CD3OD) δ 8.05 (d, J=8.2, 2H), 7.38-7.22 (m, 7H), 5.85 (s, 1H), 4.88 (s, 3H), 3.63 (d, J=12.8, 1H), 3.41 (d, J=12.8, 1H), 3.09 (s, 1H), 2.98-2.80 (m, 2H), 2.40 (s, 3H), 2.22 (dd, J=9.0, 16.2, 2H), 1.91 (d, J=4.2, 1H), 1.66-1.45 (m, 2H), 1.02 (d, J=7.1, 3H); MS (ES⁺): 219.3 (M⁺¹). Analysis: Calc for C₄₈H₆₂N₄O₈(H₂O)_{1.25} C, 68.18; H, 7.68; N, 6.62.

Found: C, 67.92; H, 7.46; N, 6.44.

f. To a stirred solution of tert-butyl hydrazinecarboxylate 11 (50 g, 412.37 mmol) and 2,5-dimethoxytetrahydrofuran 10 (54.5 g, 412.37 mmol) in dioxane (300 mL) was added aqueous hydrochloric acid (5 mL, 2N). The reaction was set up using a dean-stark apparatus and heated at 90° C. for 20 h. Reaction mixture was cooled to 20° C., neutralized with saturated sodium bicarbonate (18 mL) and filtered to remove inorganics. The filtrate was concentrated in vacuum and triturated with ether. The solid obtained was collected by filtration to furnish on drying tert-butyl 1H-pyrrol-1-ylcarbamate 12 (43 g, 57.2%) as a yellow brown solid. ¹H NMR (300 MHz, CD3OD) δ 6.62 (t, J=2.3, 2H), 6.02 (t, J=2.3, 2H), 1.48 (s, 9H); MS (ES⁺): 181.1 (M⁻¹). HPLC (Zorbax SBC3, 3.0×150 mm, 5 μm, with ZGC SBC3, 2.1×12.5 mm guard cartridge. Mobile phase: 0.1 M ammonium acetate/Acetonitrile) Rt=18.44, (100%). Analysis: Calc for C₉H₁₄N₂O₂: C, 59.32; H, 7.74; N, 15.37. Found: C, 59.32; H, 7.65; N, 15.02.
g. To a stirred solution of tert-butyl 1H-pyrrol-1-ylcarbamate 12 (40 g, 219.52 mmol), in acetonitrile (350 mL) was added chlorosulfonyl isocyanate (32.62 g, 230.50 mmol) slowly at 0° C. and continued stirring at 0° C. for 30 min. To the solution N,N-dimethyl formamide (40 mL) was added below 5° C. and continued stirring at 0° C. for 1 hr. The reaction mixture was poured into a mixture of crushed ice (1 L) and ethyl acetate (1 L). The layers were separated and the organic layer was washed with water (500 mL), brine (250 mL), dried and concentrated in vacuum to furnish crude (43 g) product. The crude was purified by flash chromatography (silica gel, eluting with ethyl acetate in hexane 0-50%) to afford pure tert-butyl 2-cyano-1H-pyrrol-1-ylcarbamate 13 (30 g, 66%) as a colorless solid. ¹H NMR (300 MHz, DMSO) δ 10.80 (s, 1H, D₂O exchangeable), 7.23 (dd, J=1.7, 2.9, 1H), 6.94 (dd, J=1.7, 4.3, 1H), 6.20 (dd, J=2.9, 4.3, 1H), 1.45 (s, 9H). HPLC (Zorbax SBC3, 3.0×150 mm, 5 μm, with ZGC SBC3, 2.1×12.5 mm guard cartridge. Mobile phase: 0.1 M ammonium acetate/Acetonitrile) Rt=16.216, (98.14%). Analysis: Calc for C₁₀H₁₃N₃O₂: C, 57.95; H, 6.32; N, 20.27. Found: C, 58.02; H, 6.45; N, 20.18.
h. To a stirred solution of tert-butyl 2-cyano-1H-pyrrol-1-ylcarbamate 13 (5 g, 24.12 mmol) in ethyl alcohol (100 ml) was added concentrated aqueous ammonium hydroxide solution (50 mL) at 20° C. followed by hydrogen peroxide (7.4 mL, 72.38 mmol, 30% in water) slowly at 20° C. and stirred at the same temperature for 16 h. Reaction mixture was concentrated in vacuum and diluted with ethyl acetate (150 mL), washed with water (2×50 mL). The aqueous layer was extracted with ethyl acetate (150 mL). The combined ethyl acetate layers were washed with water (100 mL), brine (50 mL), dried, filtered, and concentrated in vacuum. The residue obtained was crystallized from diisopropyl ether and hexane to afford tert-butyl 2-carbamoyl-1H-pyrrol-1-ylcarbamate 14 (4.0 g, 73.6%) as a colorless solid. ¹H NMR (300 MHz, DMSO) δ 9.89 (s, 1H, D₂O exchangeable), 7.31 (d, J=38.5, 1H), 6.84 (dd, J=1.9, 2.8, 2H, 1H is D₂O exchangeable), 6.76 (dd, J=1.9, 4.2, 1H), 5.97 (dd, J=2.8, 4.2, 1H), 1.40 (s, 9H). HPLC (Zorbax SBC3, 3.0×150 mm, 5 μm, with ZGC SBC3, 2.1×12.5 mm guard cartridge. Mobile phase: 0.1 M ammonium acetate/Acetonitrile) Rt=12.817, (97.6861%). Analysis: Calc for C₁₀H₁₅N₃O₃: C, 53.32; H, 6.71; N, 18.65. Found: C, 53.40; H, 6.74; N, 18.55.
i. To a solution of tert-butyl 2-carbamoyl-1H-pyrrol-1-ylcarbamate 14 (2 g, 8.87 mmol) in dichloromethane (15 ml) was added trifluoroacetic acid (15 mL) at 20° C. and stirred for 30 min. The reaction mixture was concentrated to dryness to remove excess trifluoroacetic acid and diluted with dichloromethane. Triethylorthoformate (30 mL) was added to the residue and was heated to 79° C. overnight. Reaction mixture was concentrated to dryness and triturated with hexanes, the solid obtained was collected by filtration dried in vacuum to give crude pyrrolo[1,2-f][1,2,4]triazin-4-ol 15 (1.1 g, 91%) as a dark brown solid. ¹H NMR (300 MHz, DMSO) δ 11.63 (s, 1H, D₂O exchangeable), 7.83 (d, J=4.0, 1H), 7.59 (dd, J=1.7, 2.6, 1H), 6.89 (dd, J=1.6, 4.3, 1H), 6.54 (dd, J=2.7, 4.3, 1H); MS (ES⁺): 136.2 (M+1). HPLC (SBC3, 3.0×150 mm, 5 μm, with ZGC SBC3, 2.1×12.5 mm guard cartridge. Mobile phase: 0.1 M ammonium acetate/Acetonitrile) Rt=12.817, (95.9%).
j. The stirred solution of pyrrolo[1,2-f][1,2,4]triazin-4-ol 15 (1 g, 7.40 mmol), benzyltriethylammonium chloride (3.29 g, 14.80 mmol), and N,N-dimethylaniline (1.35 g, 11.10 mmol) in acetonitrile (25 mL) was heated to 80° C. and at this temperature phosphorous oxy chloride (6.88 g, 44.40 mmol) was added and stirred at 80° C. for 16 h. The reaction was concentrated to remove Acetonitrile and phosphorus oxy chloride. The reaction was quenched by adding ice water (20 mL). Extracted with ethyl acetate (2×100 mL). The combined ethyl acetate extracts were washed with hydrochloric acid (1 N, 30 mL) water (50 mL), saturated sodium bicarbonate (1×20 mL), water (50 mL), brine (20 mL) dried and concentrated. The crude residue was purified by flash chromatography [silica gel, eluting with ethyl acetate in hexanes (0 to 5%)] to furnish pure 4-chloropyrrolo[1,2-f][1,2,4]triazine 16 (0.7 g, 61.6%) as a colorless oil, which solidified on standing in refrigerator.

¹H NMR (300 MHz, DMSO) δ 8.44 (s, 1H), 8.27 (dd, J=1.5, 2.5, 1H), 7.12 (qd, J=2.0, 4.6, 2H).

k. To a stirred suspension of bis[(1-benzyl-4-methylpiperidin-3-yl)-methylamine] di-p-toluoyl-L-tartarate 9 (0.61 g, 0.74 mmol), 4-chloropyrrolo[1,2-f][1,2,4]triazine 16 (0.227 g, 1.482 mmol) and potassium carbonate (0.61 g, 4.44 mmol) in water (5 mL) were stirred at 100° C. for 4 days. The reaction mixture was cooled to 20° C. and diluted with water (10 mL) and extracted with ethyl acetate (2×50 mL). the combined organic layers were washed with sodium hydroxide solution (1 N, 10 mL), water (10 mL), and brine (10 mL), dried and concentrated in vacuum. The crude residue was purified by flash chromatography to afford pure N-((3R,4R)-1-benzyl-4-methylpiperidin-3-yl)-N-methylpyrrolo[1,2-f][1,2,4]triazin-4-amine 17 (0.35 g, 72.1%) as a sticky syrup. ¹H NMR (300 MHz, DMSO) δ 7.77 (s, 1H), 7.68 (dd, J=1.5, 2.6, 1H), 7.32 (d, J=4.3, 4H), 7.24 (dt, J=4.4, 8.9, 1H), 6.92 (s, 1H), 6.65 (dd, J=2.7, 4.6, 1H), 5.20 (s, 1H), 3.49 (d, J=2.0, 2H), 3.33 (s, 3H), 2.82 (dd, J=5.7, 11.6, 1H), 2.67 (s, 1H), 2.55 (d, J=9.6, 1H), 2.27 (s, 1H), 2.13 (s, 1H), 1.65 (d, J=7.6, 2H), 0.91 (d, J=7.0, 3H). MS (ES⁺): 336.2 (M+1). HPLC (BCX-5101 method, Zorbax SBC3, 3.0×150 mm, 5 μm, with ZGC SBC3, 2.1×12.5 mm guard cartridge. Mobile phase: 0.1 M ammonium acetate/Acetonitrile) Rt=20.32, (96.7%).
l. To a solution of N-((3R,4R)-1-benzyl-4-methylpiperidin-3-yl)-N-methylpyrrolo[1,2-f][1,2,4]triazin-4-amine 17 (0.323, 0.964 mmol) in ethanol (10 mL) was added aqueous hydrochloric acid (2 N, 1 mL) and palladium hydroxide (0.25 g, 20 wt %, dry basis). The suspension was hydrogenated in par shaker at 50 psi for 48 hrs. The reaction mixture was diluted with methanol (50 mL) and filtered through a pad of celite and concentrated. The crude residue was purified by flash chromatography [silica gel, eluting with CMA 80 in chloroform (0 to 25%)] to furnish pure N-methyl-N-((3R,4R)-4-methylpiperidin-3-yl)pyrrolo[1,2-f][1,2,4]triazin-4-amine 18 (0.21 g, 68.6%) as a pale yellow thick syrup. ¹H NMR (300 MHz, DMSO) δ 7.80 (s, 1H), 7.68 (dd, J=1.5, 2.6, 1H), 6.89 (s, 1H), 6.66 (dd, J=2.7, 4.5, 1H), 4.91 (s, 1H), 3.47 (s, 3H), 3.33 (s, 1H), 3.14 (dd, J=8.5, 12.1, 1H), 2.81 (ddd, J=3.6, 11.0, 12.7, 2H), 2.62 (dt, J=4.5, 12.3, 1H), 2.31 (s, 1H), 1.70 (s, 1H), 1.53-1.42 (m, 1H), 0.99 (d, J=7.2, 3H); MS (ES⁺): 246.2 (M+1).

Compound 7 can be prepared as described in Organic Process Research and Development 2005, 9, 51-56. Compound 13 can be prepared as described in International Patent Application Publication Number WO2007/064931.

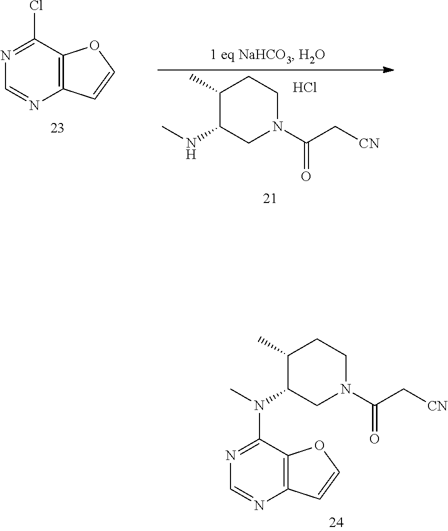

### Example 2

### 3-((3R,4R)-3-(furo[3,2-d]pyrimidin-4-yl(methyl)amino)-4-methylpiperidin-1-yl)-3-oxopropanenitrile (24)

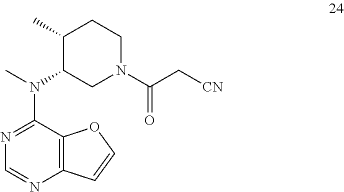

To a solution of 4-chloro-furo[3,2-d]pyrimidine 23 (0.1 g, 0.64 mmol) in dioxane (2 mL) was added 3-((3R,4R)-4-methyl-3-(methylamino)piperidin-1-yl)-3-oxopropanenitrile hydrochloride 21 (0.149 g, 0.64 mmol) in water (1 mL) and sodium bicarbonate (54 mg, 0.64 mmol) in water (5 mL). The reaction mixture was stirred at 100° C. for 1 h. After diluting with water, it was extracted with ethyl acetate (2×50 mL). The organic layers were combined and washed with water (20 mL), brine (10 mL), dried (MgSO₄), filtered and the filtrate was concentrated. The residue was purified by column chromatography (silica gel 12 g, eluting with 0-50% CMA 80 in chloroform) to furnish the desired compound 24 as a white solid. ¹H NMR (300 MHz, DMSO) (350° K) δ 8.34 (s, 1H), 8.16 (d, J=2.2, 1H), 6.92 (d, J=2.1, 1H), 4.87 (dd, J=12.0, 6.9 Hz, 1H), 4.09-3.89 (m, 2H), 3.82 (s, 2H), 3.45 (s, 2H), 3.31 (s, 3H), 2.37 (s, 1H), 1.85-1.58 (m, 2H), 1.01 (d, J=7.1 Hz, 3H). MS (ES⁺) 314.1 (100%: M⁺¹), 336.1 (30%, M+23).

### Preparation of Intermediate Compound 21

a. To a solution of bis[(1-benzyl-4-methylpiperidin-3-yl)-methylamine] di-p-toluoyl-L-tartrate 9 (16.46 g, 40 mmol) in dioxane/water (2:1) (100 mL) was added 2N NaOH (32 mL, 64 mmol)) and boc anhydride (9.82 g, 44 mmol). The reaction was stirred at room temperature overnight and concentrated in vacuo to remove dioxane. The reaction mixture was diluted with water (50 mL) and extracted twice with ethyl acetate (150 mL). The organic layers were combined washed with brine (100 mL), dried over MgSO₄ and filtered. The filtrate was concentrated in vacuo and the residue obtained was purified by flash column chromatography (silica gel, 240 g eluting with ethyl acetate in hexanes 0-40%) to furnish tert-butyl (3R,4R)-1-benzyl-4-methylpiperidin-3-yl(methyl)carbamate (10.45 g, 82%) as colorless oil. ¹H NMR (300 MHz, DMSO) δ 7.47-7.18 (m, 5H), 4.03 (d, J=7.1 Hz, 1H), 3.42 (q, J=13.1 Hz, 2H), 3.01 (s, 3H), 2.66 (m, 2H), 2.36 (m, 1H), 2.12 (m, 1H), 1.86 (m, 1H), 1.51 (m, 2H), 1.37 (s, 9H), 0.86 (d, J=7.0 Hz, 3H); MS (ES⁺): 319.2 (100%, M⁺¹). Analysis: Calc for C₁₉H₃₀N₂O₂.0.25 H₂O: C, 70.66; H, 9.52; N, 8.67. Found: C, 70.72; H, 9.43; N, 8.65.
b. To a solution of tert-butyl (3R,4R)-1-benzyl-4-methylpiperidin-3-yl(methyl)carbamate (10 g, 31.4 mmol) in ethanol (200 mL) was added Pd/C (10% on carbon, 1.5 g) and hydrogenated on the Parr Shaker at 60 psi for 72 h. The reaction mixture was filtered through a pad of Celite and the filtrate was concentrated in vacuo to furnish tert-butyl ((3R,4R)-4-methylpiperidin-3-yl)carbamate (6.17 g, 87%) as a colorless oil. ¹H NMR (300 MHz, DMSO) δ 3.89 (s, 1H), 3.44 (q, J=7.0 Hz, 1H), 3.00-2.85 (m, 4H), 2.72 (dd, J=4.1 Hz, 12.2, 2H), 2.53 (d, J=15.0 Hz, 1H), 2.03 (m, 1H), 1.51 (m, 1H), 1.39 (s, 9H), 1.06 (t, J=7.0 Hz, 1H), 0.90 (d, J=7.2 Hz, 3H); MS (ES⁺): 229.2 (100%, M⁺¹).
c. To a solution of tert-butyl ((3R,4R)-4-methylpiperidin-3-yl)carbamate (5.64 g, 24 7 mmol) in methylene chloride (150 mL) cooled to 0° C. was added cyanoacetic acid (3.4 g, 40 mmol), EDCI (7.67 g, 40 mmol), and triethylamine (5.6 mL, 40 mmol). The reaction was allowed to warm to room temperature overnight. The reaction mixture was washed with water (150 mL), brine (100 mL), dried over MgSO₄, and concentrated in vacuo. The residue obtained was purified by flash column chromatography (silica gel 150 g, eluting with flash with ethyl acetate in hexanes 0-50%) to give tert-butyl (3R,4R)-1-(2-cyanoacetyl)4-methylpiperidin-3-yl(methyl)carbamate (3.6 g, 50%) as a white solid. ¹H NMR (300 MHz, DMSO) δ 4.16-4.01 (m, 2H), 4.00-3.85 (m, 1H), 3.71 (dd, J=6.9, 13.3, 1H), 3.66-3.38 (m, 2H), 3.25 (d, J=4.4, 1H), 2.75 (d, J=7.2, 3H), 2.10 (s, 1H), 1.69-1.44 (m, 2H), 1.40 (s, 9H), 0.93 (d, J=7.1, 3H); MS (ES⁺): 613.3 (100%, 2M^{+Na}).
d. To a solution of tert-butyl (3R,4R)-1-(2-cyanoacetyl)-4-methylpiperidin-3-yl(methyl)carbamate (2.66 g, 9 mmol) in THF (22.5 mL) was added 4M HCl in dioxane (22.5 mL, 9 mmol). The reaction was stirred at room temperature overnight. The solid obtained was collected by filtration washed with ether and dried in vacuo to give 3-((3R,4R)-4-methyl-3-(methylamino)piperidin-1-yl)-3-oxopropanenitrile hydrochloride 21 (1.95 g, 94%) as a white solid. ¹H NMR (300 MHz, DMSO) δ 9.64-8.23 (m, 2H, exchangeable), 4.31 (dd, J=3.0, 10.8 Hz, 1H), 4.03 (m, 2H), 3.55 (m, 1H), 3.25 (m, 1H), 3.16 (m, 2H), 2.63 (d, J=8.0 Hz, 3H), 2.14 (m, 1H), 1.57 (m, 2H), 1.04 (d, J=7.8 Hz, 3H); MS (ES⁺): 196.3 (100%, M⁺¹).

Preparation of Intermediate Compound 23.

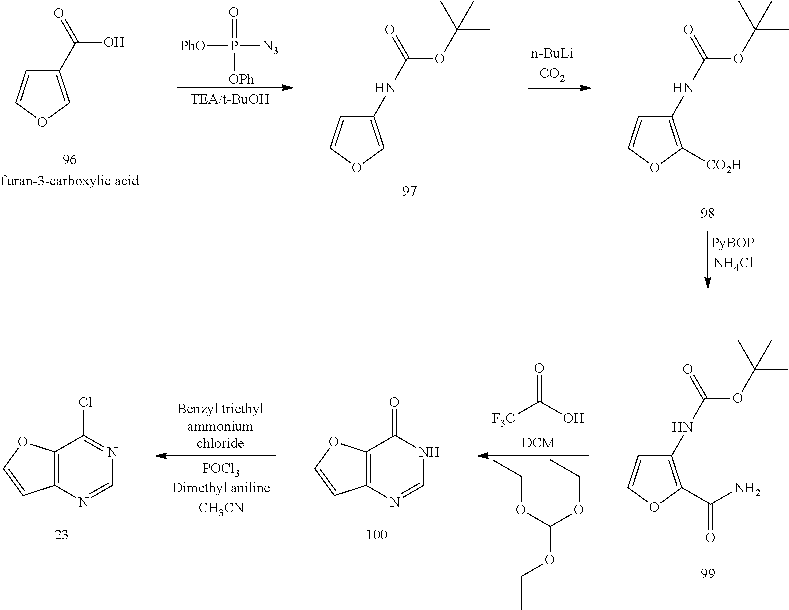

e. To a solution of 3-furoic acid 96 (54.4 g, 485 mmol), triethylamine (105 ml, 753 mmol), tert-butanol (25.2 mL, 786 mmol) in toluene (800 mL) was added dropwise at room temperature over 45 min period diphenyl phosphoryl azide (157.8 mL, 732 mmol). The resulting solution was heated at reflux for 6 h and at room temperature overnight. The reaction was diluted with water (1000 mL) and extracted twice with ethyl acetate (1000 ml). The organic layers were combined washed with water (800 mL), brine (800 mL), decolorized with activated charcoal, dried, filtered and concentrated in vacuo to furnish a brown semisolid. The semisolid was crystallized from dichloromethane (300 mL) and hexanes (600 mL) to furnish tert-butyl furan-3-ylcarbamate 97 (61.5 g, 78%). ¹H NMR (300 MHz, CDCl₃) δ 7.71 (s, 1H), 7.30-7.24 (m, 1H), 6.43 (s, 1H), 6.27 (s, 1H), 1.75-1.32 (s, 9H).
f. To a solution of tert-butyl furan-3-ylcarbamate 97 (5.49 g, 30 mmol) in THF (60 mL) cooled to −40° C. was added n-butyl lithium (1.6 M, 45 mL, 72 mmol) dropwise. The reaction was stirred at −40° C. for 4 h and quenched into dry CO₂ (100 mL) in ether (300 mL). The reaction mixture was poured into water (300 mL) with stirring and the aqueous layer was separated. The aqueous layer was washed with ether (100 mL). The combined organic layer was extracted with water (2×100 mL). The aqueous layers were combined acidified with cone. HCl and extracted with ethyl acetate (3×200 mL). The ethyl acetate layers were combined dried, filtered and concentrated in vacuo to furnish yellow solid (5.48 g). The yellow solid was triturated with hexanes and solid obtained was collected by filtration to furnish 3-(tert-butoxycarbonylamino)furan-2-carboxylic acid 98 (3.6 g, 53%) as a light yellow solid. ¹H NMR (300 MHz, DMSO) δ 13.23 (s, 1H), 8.35-8.23 (m, 1H), 7.77 (t, J=10.0, 1H), 7.07 (s, 1H), 1.53-1.40 (m, 9H).
g. To a solution of 2-(tert-butoxycarbonylamino)furan-3-carboxylic acid 98 (1.0 g, 4.4 mmol), in DMF (15 mL0) was added DIPEA (3.8 g, 22 mmol), PyBOP (2.75 g, 5.28 mmol) and ammonium chloride (0.47 g, 8.8 mmol) and stirred at room temperature for 2 h. The reaction was poured into 0.4 M aqueous HCl (70 mL) and extracted with dichloromethane (3×50 mL). The combined organic layers were washed with water (40 mL), brine (40 mL), dried, filtered and concentrated in vacuo. The residue obtained was purified by flash column chromatography (silica gel, 20 g, eluting with 0 to 100% ethyl acetate in hexane) yielding tert-butyl 2-carbamoylfuran-3-ylcarbamate 99 (0.75 g, 75%) as a white solid: MP 140-143° C. ¹H NMR (300 MHz, DMSO) δ 8.85 (s, 1H), 7.85-7.45 (m, 3H), 7.04 (s, 1H), 1.57-1.39 (m, 9H)
h. To a solution of tert-butyl 2-carbamoylfuran-3-ylcarbamate 99 (2.47 g, 10.87 mmol) in dichloromethane (20 mL) was added trifluoroacetic acid (20 mL) and stirred at room temperature for 30 min. The reaction mixture was concentrated in vacuo and the residue was suspended in triethyl orthoformate (40 mL) and refluxed at 80° C. for 5 h. The reaction mixture was concentrated in vacuo and the white solid obtained was titurated with ether (250 mL) and collected by filtration to furnish furo[3,2-d]pyrimidin-4(3H)-one 100 (1.547 g, 100%) as a solid on drying in vacuo. ¹H NMR (300 MHz, DMSO) δ 12.87-12.25 (m, 1H), 8.23 (d, J=2.1 Hz, 1H), 8.07 (s, 1H), 7.00 (d, J=2.1 Hz, 1H).
i. To a solution of above furo[3,2-d]pyrimidin-4(3H)-one 100 (1.547 g, 11.37 mmol), benzyltriethyl ammonium chloride (5.18 g, 22.73 mmol) and dimethyl aniline (2.16 mL, 17.06 mmol) in acetonitrile (40 mL) at 80° C. was added phosphorous oxychloride (6.6 mL) and stirred at 80° C. for 4 h. The reaction mixture was concentrated in vacuo and quenched with ice-cold water and stirred for 0.5 h. The aqueous layer was extracted with ethyl acetate (2×100 mL). The organic layers were combined, washed with 1N HCl (150 mL), saturated NaHCO₃ solution (150 mL), brine (150 mL), dried over MgSO₄, filtered and concentrated in vacuo to furnish crude product. The crude product was purified by flash column chromatography (silica gel, 40 g, eluting with 0-100% [9:1] ethyl acetate/methanol in hexanes) to furnish 23 (0.836 g, 50%) as an off-white solid; mp 122.5° C.; ¹H NMR (300 MHz, DMSO) δ 8.92 (s, 1H), 8.68 (d, J=2.3 Hz, 1H), 7.40 (d, J=2.3 Hz, 1H).

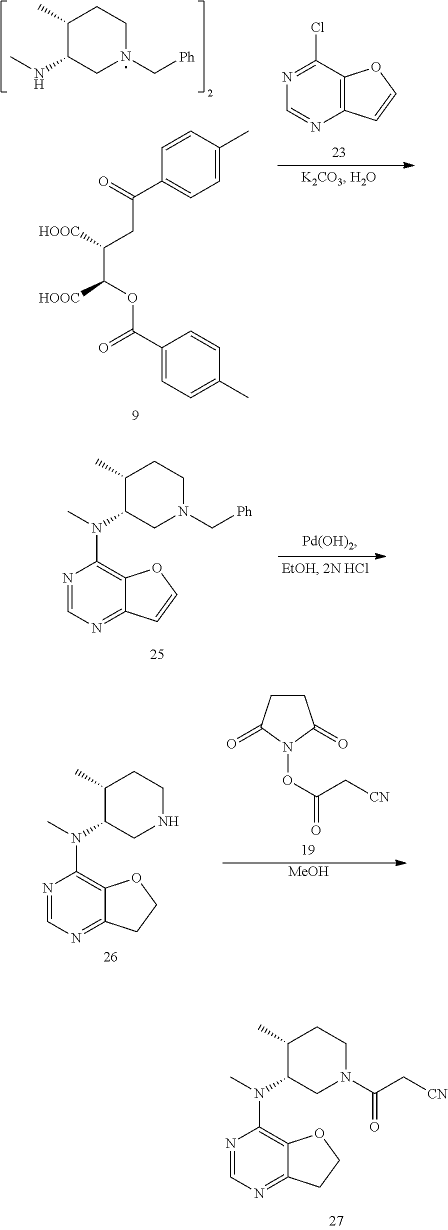

### Example 3

### 3-((3R,4R)-3-((6,7-dihydrofuro[3,2-d]pyrimidin-4-yl)(methyl)amino)-4-methylpiperidin-1-yl)-3-oxopropanenitrile (27)

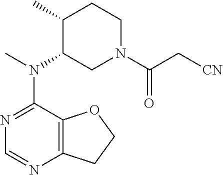

To a stirred suspension of cyano acetic acid (5 g, 58.78 mmol) and N-hydroxysuccinimide (6.76 g, 58.78 mmol) in dichloromethane (100 mL) was added dicychohexyl carbodiimide (12.12 g, 58.78 mmol) at 0° C. The reaction was stirred for 18 hrs at 20° C. The solid separated was filtered and the filtrate was concentrated to afford crude 2,5-dioxopyrrolidin-1-yl 2-cyanoacetate (19) (6.5 g, crude). This was used as such in next step.

To a solution of N-methyl-N-((3R,4R)-4-methylpiperidin-3-yl)-6,7-dihydrofuro[3,2-d]pyrimidin-4-amine (26) (0.089 g, 0.35 mmol) in methanol (5 mL) was added at room temperature 2,5-dioxopyrrolidin-1-yl 2-cyanoacetate (0.32 g) and stirred for 18 h. Reaction mixture was concentrated in vacuum to remove methanol and the residue obtained was suspended in Ethyl acetate (20 mL) and filtered. The filtrate was washed with water (20 mL), brine (20 mL), dried and concentrated in vacuum. The residue obtained was purified by flash chromatography [silica gel, eluting with ethyl acetate and methanol (9:1) in hexanes 0 to 50%] to afford 3-((3R,4R)-3-((6,7-dihydrofuro[3,2-d]pyrimidin-4-yl)(methyl)amino)-4-methylpiperidin-1-yl)-3-oxopropanenitrile (27) (46 mg, 41.7%) as a colorless solid. ¹HNMR (300 MHz, DMSO) δ 8.13 (s, 1H), 4.63-4.46 (m, 3H), 3.79-3.68 (m, 2H), 3.50-3.21 (m, 5H), 3.09 (2s, 3H), 2.31-2.18 (m, 1H), 1.76-1.65 (m, 1H), 1.63-1.49 (m, 2H), 0.97 (2d, J=5.6, 3H); IR (KBr): 2254 cm⁻¹; MS (ES⁺): 316.1 (M+1), 338.1 (M+23).

Preparation of Intermediate Compound (26)

a. A stirred suspension of bis[(1-benzyl-4-methylpiperidin-3-yl)-methylamine] di-p-toluoyl-L-tartarate (9) (0.88 g, 1.06 mmol), 4-chlorofuro[3,2-d]pyrimidine (23) (0.33 g, 2.13 mmol) and potassium carbonate (0.945 g, 6.84 mmol) in water (10 mL) was heated at 100° C. for 20 h. The reaction mixture was cooled and diluted with water (10 mL). The aqueous layer was extracted with ethyl acetate (2×50 mL). The organic layer were combined washed with saturated aqueous sodium hydrogen carbonate solution (10 mL), water (10 mL), and brine (10 mL), dried and concentrated in vacuum. The crude residue obtained was purified by flash chromatography to afford N-((3R,4R)-1-benzyl-4-methylpiperidin-3-yl)-N-methylfuro[3,2-d]pyrimidin-4-amine (25) (0.35 g, 72.1%) as an oil. ¹HNMR (300 MHz, CDCl₃) δ 8.41 (s, 1H), 7.68 (d, J=2.2, 1H), 7.35-7.22 (m, 5H), 6.80 (d, J=2.2, 1H), 5.05 (t, J=24.2, 1H), 3.61 (s, 3H), 3.56-3.43 (m, 2H), 2.88 (dd, J=5.2, 11.7, 1H), 2.80-2.68 (m, 1H), 2.60 (dd, J=4.1, 11.7, 1H), 2.35-2.24 (m, 1H), 2.20-2.07 (m, 1H), 1.83-1.62 (m, 2H), 0.93 (d, J=7.0, 3H); MS (ES⁺): 337.2 (M+1); Analysis: Calcd for C₂₀H₂₄N₄O.0.25 H₂O: C, 70.45; H, 7.23; N, 16.43. Found: C, 70.08; H, 7.23; N, 15.46.
b. To a solution of N-((3R,4R)-1-benzyl-4-methylpiperidin-3-yl)-N-methylfuro[3,2-d]pyrimidin-4-amine (25) (0.3 g, 0.89 mmol) in ethanol (10 mL) was added aqueous hydrochloric acid (2 N, 1 mL) and palladium hydroxide (0.25 g, 20 wt %, dry basis). The suspension was hydrogenated in a parr shaker at 50 psi for 16 h. The reaction mixture was diluted with methanol (50 mL), filtered through a pad of celite to remove the catalyst and the filtrate was concentrated in vacuum. The crude residue obtained was purified by flash chromatography (silica gel, eluting with CMA 80 in chloroform 0 to 25%) to afford N-methyl-N-((3R,4R)-4-methylpiperidin-3-yl)-6,7-dihydrofuro[3,2-d]pyrimidin-4-amine (26) (0.180 g, 81.2%) as a pale yellow syrup. ¹H NMR (300 MHz, DMSO) δ 8.20-8.01 (m, 1H), 4.59-4.41 (m, 3H), 3.20 (s, 3H), 3.14-3.01 (m, 3H, D₂O exchangeable, 1H), 2.85-2.70 (m, 2H), 2.62-2.52 (m, 1H), 2.14 (td, J=5.7, 11.8, 2H), 1.61 (ddt, J=4.4, 9.1, 13.5, 1H), 1.43 (dtd, J=3.4, 5.7, 9.1, 1H), 0.92 (d, J=7.2, 3H); MS (ES⁺): 249.2 (M+1).

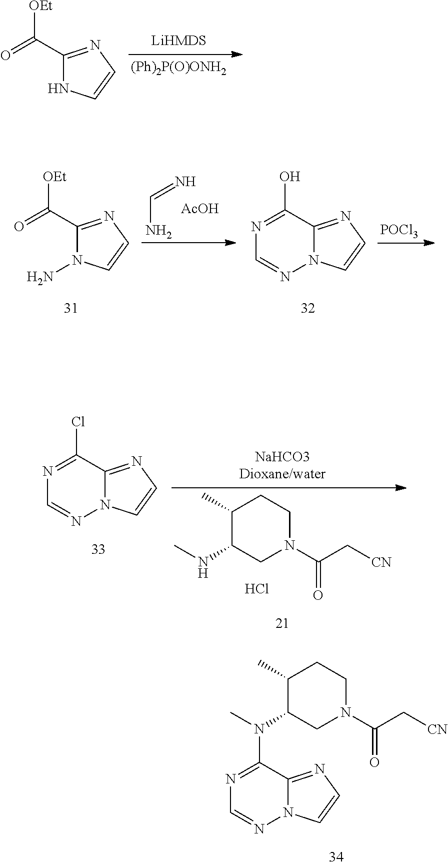

### Example 4

### 3-((3R,4R)-3-(Imidazo[1,2-f][1,2,4]triazin-4-yl(methyl)amino)-4-methylpiperidin-1-yl)-3-oxopropanenitrile (34)

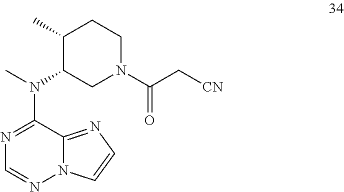

To a solution of 4-chloroimidazo[1,2-f][1,2,4]triazine (33) (0.23 mg, 1 mmol) and 3-((3R,4R)-4-methyl-3-(methylamino)piperidin-1-yl)-3-oxopropanenitrile (21) (0.15 g, 1 mmol) in dioxane water (3:8 mL) was added NaHCO₃ (0.084 g, 1 mmol). The mixture was heated in a microwave at 100° C. for 30 min and concentrated in vacuum. The residue obtained was purified by flash column chromatography (silica gel 12 g, eluting with 0-20% CMA-80 in chloroform) to furnish 3-((3R,4R)-3-(Imidazo[1,2-f][1,2,4]triazin-4-yl(methyl)amino)-4-methylpiperidin-1-yl)-3-oxopropanenitrile (34) (0.05 g, 16%) as an off-white solid; mp 72.0° C. ¹H NMR (300 MHz, DMSO) δ 8.11 (s, 1H), 8.05 (s, 1H), 7.62 (s, 1H), 6.45-5.73 (m, 1H), 4.08 (s, 2H), 3.87 (s, 3H), 3.40 (s, 3H), 2.47-2.34 (m, 1H), 1.89-1.50 (m, 2H), 1.01 (s, 3H); MS 314.1 (100%, M+1, ES⁺).

Preparation of Intermediate Compound (33)

a. To a solution of ethyl 1H-imidazole-2-carboxylate (Aldrich, 3.0 g, 21.40 mmol) in anhydrous DMF (10 mL) at −10° C. was added dropwise Lithium hexamethyldisilazane (1.10 mL, 1 M solution in THF, 1.1 mmol). After the mixture was stirred for 10 min, diphenylphosphinyl)hydroxylamine (6.49 g, 27.83 mmol) was added at 0° C., followed by stirring at room temperature overnight. The reaction was quenched with water until a clear solution was formed and concentrated in vacuum to dryness. The residue obtained was extracted with ethyl acetate (5×100). The organic extracts were combined and concentrated in vacuo to furnish ethyl 1-amino-1H-imidazole-2-carboxylate (31) (3.1 g, 94%) as a brown oil. This was pure enough to be used in next step.
b. A mixture of above ethyl 1-amino-1H-imidazole-2-carboxylate (31) (3.1 g) and formamidine acetate (11.16 g, 107.2 mmol) in ethanol was heated at reflux overnight. The reaction mixture was concentrated in vacuum to dryness, diluted with water (75 mL) and extracted with ethyl acetate (2×75 mL). The ethyl acetate layers were combined and concentrated in vacuum to furnish imidazo[1,2-f][1,2,4]triazin-4-ol (32)(2 g, 68.7%) as a white solid. This was pure enough to be used for next step. ¹HNMR (300 MHz, DMSO) δ 12.34 (s, 1H), 8.11 (s, 1H), 8.00 (d, J=1.1, 1H), 7.52 (d, J=1.1, 1H).
c. A stirred solution of imidazo[1,2-f][1,2,4]triazin-4-ol (32) (0.5 g, 3.67 mmol) in phosphorous oxy chloride (15 mL) was heated at reflux for 16 h. The reaction was concentrated to remove phosphorus oxy chloride, quenched by adding ice water (20 mL) and extracted with ethyl acetate (2×50 mL). The ethyl acetate extracts were combined and washed with saturated sodium bicarbonate (20 mL), water (20 mL); brine (20 mL) dried and concentrated in vacuum. The crude residue obtained was purified by flash chromatography (silica gel, eluting with ethyl acetate in hexanes 0 to 5%) to furnish 4-chloroimidazo[1,2-f][1,2,4]triazine (33) (0.34 g, 60%) as a brown solid. ¹HNMR (300 MHz, DMSO) δ 8.81 (s, 1H), 8.65 (d, J=1.1, 1H), 8.08 (d, J=1.0, 1H).

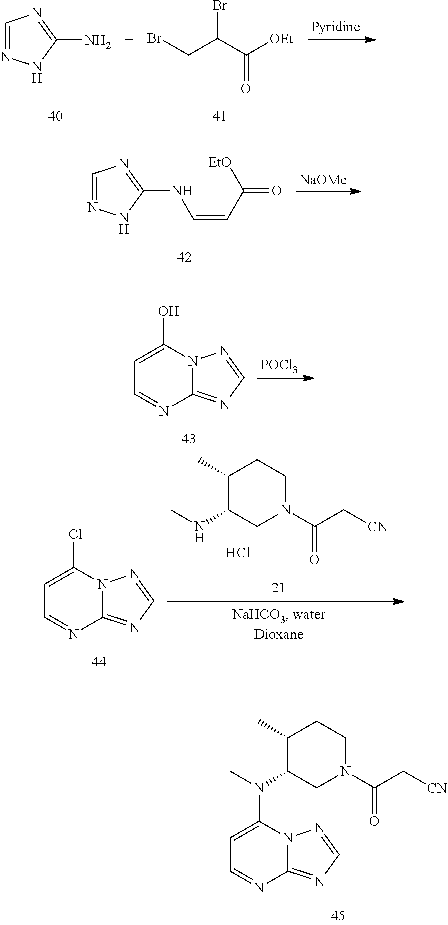

### Example 5

### 3-((3R,4R)-3-([1,2,4]-Triazolo[1,5-a]pyrimidin-7-ylmethyl)amino)-4-methylpiperidin-1-yl)-3-oxopropanenitrile (45)

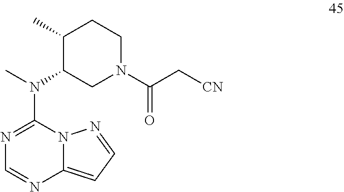

To a stirred solution of 7-chloro-[1,2,4]triazolo[1,5-a]pyrimidine (44) (0.2 g, 1.29 mmol) in dioxane (5 mL) was added 3-((3R,4R)-4-methyl-3-(methylamino)piperidin-1-yl)-3-oxopropanenitrile (21) (0.299 g, 1.23 mmol), sodium hydrogen carbonate (0.108 g, 1.29 mmol) and water (5 mL). The reaction mixture was subjected to microwave irradiation (100° C., Power Max, Power 75w) for 30 min. The reaction mixture was concentrated in vacuum and the residue obtained was purified by flash chromatography (silica gel 24 g, eluting with CMA 80 in chloroform 0 to 100%). The product obtained was repurified by flash chromatography [silica gel 12 g, eluting with ethyl acetate and methanol (9:1) in hexanes 0 to 100%] to afford 3-((3R,4R)-3-([1,2,4]Triazolo[1,5-a]pyrimidin-7-yl(methyl)amino)-4-methylpiperidin-1-yl)-3-oxopropanenitrile (45) (25 mg, 6.18%) as a colorless solid. ¹H NMR (300 MHz, DMSO) δ 8.85 (d, J=7.8, 1H), 8.18 (s, 1H), 6.87-6.75 (m, 1H), 4.61 (s, 1H), 4.21-4.02 (m, 2H), 3.91 (dd, J=13.7, 22.8, 1H), 3.84-3.53 (m, 2H), 3.41 (d, J=5.0, 1H), 3.06 (2S, 3H), 2.30 (d, J=19.2, 1H), 1.84 (d, J=6.4, 1H), 1.57 (d, J=8.8, 1H), 0.99 (2d, J=7.0, 3H); MS (ES⁺): 314.1 (M+1), 336.1 (M+23), (ES⁻): 312.0 (M−1).

Preparation of Intermediate Compound (44)

a. To a stirred solution of 1H-1,2,4-triazol-5-amine (40) (17 g, 202.18 mmol) in pyridine (100 mL) was added ethyl 2,3-dibromopropanoate (41) (52.5 g, 202.18 mmol) and heated at reflux for 4 h. The reaction mixture was cooled to room temperature and diluted with water (150 mL). The solid obtained was collected by filtration to afford on drying in vacuum Ethyl 3-(1H-1,2,4-triazol-5-ylamino)acrylate (42) (5 g, 27.4%) as a cream colored solid. ¹HNMR (300 MHz, DMSO) δ 8.20 (dd, J=1.1, 13.3, 1H), 7.63 (dd, J=3.1, 15.1, 1H), 7.43-7.23 (m, 2H, D₂O exchangeable), 6.07 (t, J=13.3, 1H), 4.25-4.08 (m, 2H), 1.24 (t, J=7.1, 3H); MS (ES⁺): 183.2 (M+1).
b. To a stirred solution of Ethyl 3-(1H-1,2,4-triazol-5-ylamino)acrylate (42) (2.98 g, 16.35 mmol) in methanol (45 mL) was added sodium methoxide (14 mL, 65.4 mmol, 25% solution in methanol) and stirred at room temperature for 18 h. The solid obtained was collected by filtration to afford on drying in vacuum [1,2,4]triazolo[1,5-a]pyrimidin-7-ol (43) (1.9 g, 85.4%) as a white solid. ¹HNMR (300 MHz, DMSO) δ 8.13 (d, J=6.0, 1H), 7.72 (s, 1H), 5.77 (d, J=7.4, 1H); MS (ES⁻): 135.0 (M−1).
c. A solution of [1,2,4]triazolo[1,5-a]pyrimidin-7-ol (43) (1 g, 7.34 mmol) in phosphorous oxy chloride (22.53 g, 146.93 mmol) was heated at reflux for 6 h. The reaction was cooled to room temperature and concentrated in vacuum to dryness. The residue obtained was dissolved in chloroform (50 mL) and washed with cold water (50 mL). The aqueous layer was extracted with chloroform (2×100 mL). The organic layers were combined washed with water (100 mL), brine (50 mL), dried and concentrated in vacuum to afford 7-chloro-[1,2,4]triazolo[1,5-a]pyrimidine (44) (0.3 g 26.4%) as a colorless solid. ¹H NMR (300 MHz, DMSO) δ 9.48 (d, J=7.1, 1H), 8.72 (s, 1H), 7.53 (d, J=7.1, 1H).

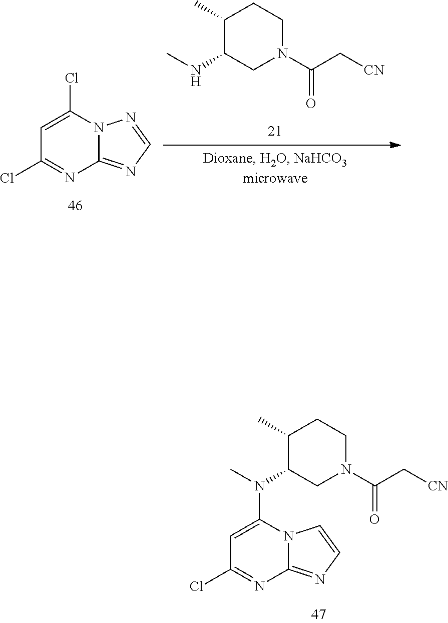

### Example 6

### 3-((3R,4R)-3-((7-Chloroimidazo[1,2-a]pyrimidin-5-yl)(methyl)amino)-4-methylpiperidin-1-yl)-3-oxopropanenitrile (47)

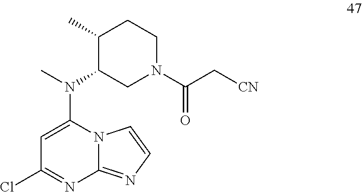

To a stirred solution of 5,7-dichloroimidazo[1,2-a]pyrimidine (46) (0.082 g, 0.43 mmol) in dioxane (2 mL) was added 3-((3R,4R)-4-methyl-3-(methylamino)piperidin-1-yl)-3-oxopropanenitrile (21) (0.10 g, 0.43 mmol), sodium hydrogen carbonate (0.036 g, 0.43 mmol) and water (2 mL). The mixture was subjected microwave irradiation (100° C., Power Max, Power 50 w) for 30 minutes. The reaction mixture was concentrated in vacuum and the residue obtained was purified by flash chromatography (silica gel 12 g, eluting with CMA-80 in chloroform 0 to 100%). The product obtained was repurified by flash chromatography [silica gel 12 g, eluting with a mixture of ethyl acetate and methanol (9:1) in hexanes (0 to 100%)] to afford 3-((3R,4R)-3-((7-Chloroimidazo[1,2-c]pyrimidin-5-yl)(methyl)amino)-4-methylpiperidin-1-yl)-3-oxopropanenitrile (47) (14 mg, 9.38%) as a colorless solid. ¹HNMR (300 MHz, DMSO, 380K) δ 7.68 (d, J=1.6, 1H), 7.61 (d, J=1.6, 1H), 6.65 (s, 1H), 3.93 (d, J=5.1, 2H), 3.88-3.79 (m, 2H), 3.65 (dd, J=8.3, 13.7, 1H), 3.46 (d, J=35.6, 2H), 3.00 (s, 3H), 2.32 (d, J=6.9, 1H), 1.80-1.58 (m, 2H), 1.03 (d, J=7.0, 3H); MS (ES⁺): 347.1 (M+1), 369.0 (M+23).

Compound 46 is commercially available from Toronto Research Chemicals, or it can be prepared as described by, Revankar, Ganaphthi R. et al., Journal of Medicinal Chemistry, 1975, 18(12); or G. R. Revankar and R. K. Robins, Ann. N.Y. Acad. Sci., 1975, 255, 166.

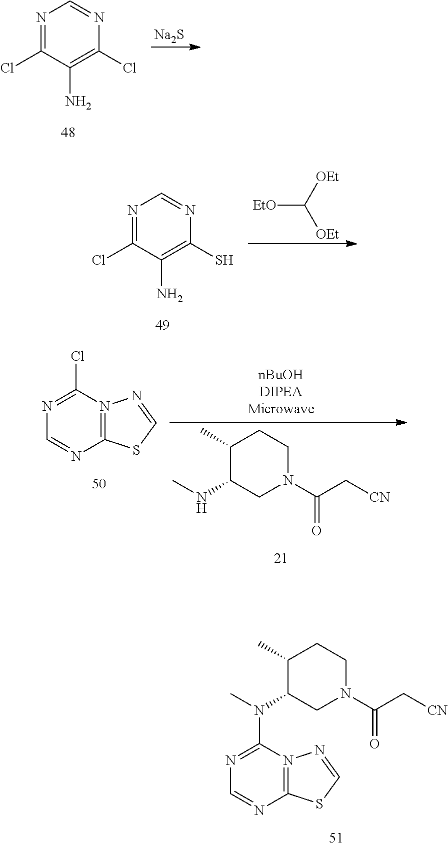

### Example 7

### 3-((3R,4R)-4-Methyl-3-(methyl(thiazolo[5,4-d]pyrimidin-7-yl)amino)piperidin-1-yl)-3-oxopropanenitrile (51)

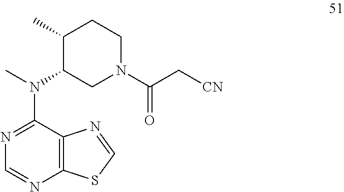

A mixture of 7-chlorothiazolo[5,4-d]pyrimidine (50) (0.171 g, 1.0 mmol) 3-((3R,4R)-4-methyl-3-(methylamino)piperidin-1-yl)-3-oxopropanenitrile (21) (0.255 g, 1.1 mmol) and DIPEA (0.7 mL, 4 mmol) in n-butanol (2 mL) was heated in a microwave at 125° C. for 30 min. The reaction mixture was concentrated in vacuo and purified by flash column chromatography [silica gel 12 g, eluting with 0-100% ethyl acetate/methanol (9:1) in hexanes] to furnish 3-((3R,4R)-4-Methyl-3-(methyl(thiazolo[5,4-d]pyrimidin-7-yl)amino)piperidin-1-yl)-3-oxopropanenitrile (51) (0.11 g, 33%) as a beige solid. ¹HNMR (300 MHz, DMSO) δ 9.23 (d, J=1.4, 1H), 8.45 (d, J=3.1, 1H), 5.40 (s, 1H), 4.19-4.03 (m, 3H), 4.01-3.90 (m, 1H), 3.88-3.66 (m, 2H), 3.42 (dd, J=4.6, 15.7, 3H), 2.41 (d, J=6.5, 1H), 1.86-1.52 (m, 2H), 1.03 (2d′ s, J=7.2, 3H); MS 364.5 (100%, M+Cl; ES⁻); HPLC [Zorbax SBC3, 3.0×150 mm, 5 μm, with a ZGC SBC3, 2.1×12 5 mm guard cartridge, “A” Buffer=(98% of 0.1 M Ammonium Acetate in 2% acetonitrile) “B” Buffer=100% Acetonitrile, UV Absorbance; R_{t=15.984} (97.87%)]; Analysis: Calcd for C₁₅H₁₈N₆OS.0.25 H₂O: C, 53.79; H, 5.57; N, 25.09; S, 9.57. Found: C, 53.73; H, 5.63; N, 24.86; S, 9.72.

Preparation of Intermediate Compound (50)

a. To a stirred solution of 5-amino-4,6-dichloropyrimidine (48) (5 gm, 30.5 mmol) in DMSO (40 ml) was added sodium sulfide (4.8 gm, 36.9 mmol) and stirred at room temperature for 12 h. The reaction mixture was diluted with water (40 ml) and acidified with conc. HCl (1 ml). The solid obtained was collected by filtration washed with water and dried in vacuum to furnish 5-amino-6-chloropyrimidine-4-thiol (49) (4.09 gm, 83.13%) as a brown solid, which was pure enough to be used for next step.
b. A solution of 5-amino-6-chloropyrimidine-4-thiol (49) (4 gm, 24.75 mmol) in triethylorthoformate was heated to reflux for 1 h. The reaction mixture was concentrated to 60% of the original volume and cooled in a refrigerator. The solid obtained was collected by filtration and dried in vacuum to furnish 7-chlorothiazolo[5,4-d]pyrimidine (50) (2.8 g, 66.04%) as a tan solid. ¹H NMR (300 MHz CDCl₃): δ 99.22 (s, 1H), 8.97 (s, 1H).

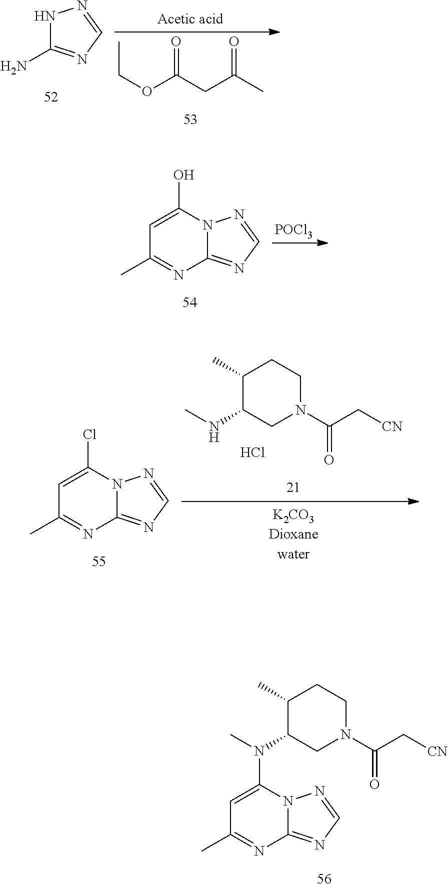

### Example 8

### 3-((3R,4R)-4-Methyl-3-(methyl(5-methyl-1,2,41-triazolo[1,5-a]pyrimidin-7-yl)amino)piperidin-1-yl)-3-oxopropanenitrile (56)

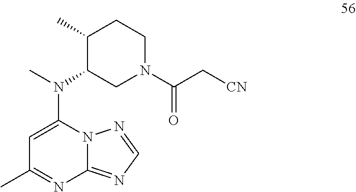

To a solution of 7-chloro-5-methyl-[1,2,4]triazolo[1,5-a]pyrimidine (55) (0.145 g, 0.865 mmol) in dioxane (2 mL) was added 3-((3R,4R)-4-methyl-3-(methylamino)piperidin-1-yl)-3-oxopropanenitrile hydrochloride (21) (0.2 g, 0.86 mmol), potassium carbonate (0.119 g, 0.86 mmol), water (5 mL) and heated with stirring at 100° C. for 4 h. The reaction mixture was diluted with water (10 mL) and extracted with ethyl acetate (2×100 mL). The organic layers were combined washed with water (20 mL), brine (10 mL), dried and concentrated in vacuum. The crude residue obtained was purified by flash column chromatography (silica gel 12 g, eluting with 0-50% CMA 80 in chloroform) to afford (56) which was re-crystallized from ethyl acetate to furnish 3-((3R,4R)-4-Methyl-3-(methyl(5-methyl-[1,2,4]triazolo[1,5-a]pyrimidin-7-yl)amino)piperidin-1-yl)-3-oxopropanenitrile (56) (18 mg, 6.35%) as a white solid; mp 119.3° C. ¹HNMR (300 MHz, DMSO) δ 8.37 (2s, 1H), 6.43 (2s, 1H), 5.26-5.04 (m, 1H), 4.22-4.02 (m, 2H), 3.93-3.72 (m, 2H), 3.67-3.40 (m, 1H), 3.30-3.14 (m, 1H), 3.11 (2s, 3H), 2.47 (2s, 3H), 2.40-2.27 (m, 1H), 1.79-1.48 (m, 2H), 1.05 (2d, J=7.2, 3H); MS (ES⁺) 328.2 (100%: M⁺¹); HPLC [(Zorbax SBC3, 3.0×150 mm, 5 μm, with a ZGC SBC3, 2.1×12.5 mm guard cartridge, “A” Buffer=(98% of 0.1 M Ammonium Acetate in 2% acetonitrile) “B” Buffer=100% Acetonitrile, UV Absorbance; Rt=26.69, (99.51%); Analysis: Calcd for C₁₆H₂₁N₇O.0.25 H₂O: C, 57.90; H, 6.52; N, 29.54. Found: C, 57.95; H, 6.48; N, 29.29.

Preparation of Intermediate Compound (55)

a. A solution of ethylacetoacetate (53) (23.21 g, 178.40 mmol) and 1H-1,2,4-triazol-5-amine (52) (15 g, 178.40) in acetic acid (90 mL) was heated at reflux for 18 h. The reaction mixture was cooled to room temperature and diluted with water (100 mL). The solid obtained was collected by filtration and dried in vacuum to afford 5-methyl-[1,2,4]triazolo[1,5-a]pyrimidin-7-ol (54) (12.5 g, 46.6%) as a colorless solid. ¹H NMR (300 MHz, DMSO) δ 13.21 (s, 1H, D₂O exchangeable), 8.16 (d, J=20.0, 1H), 5.82 (t, J=10.0, 1H), 2.42-2.21 (m, 3H); MS (ES⁺), 173.1 (M+Na), (ES⁻): 185.0 (M+Cl); Analysis: Calcd for C₆H₆N₄O: C, 47.99; H, 4.02; N, 37.31. Found: C, 47.62; H, 3.80; N, 37.11

b. A solution of 5-methyl-[1,2,4]triazolo[1,5-a]pyrimidin-7-ol (54) (2 g, 13.32 mmol) in phosphorous oxy chloride (8.23 g, 53.64 mmol) was heated at reflux for 1.5 h. The reaction mixture was cooled to room temperature and concentrated in vacuum to dryness. The residue obtained was quenched by adding ice water and extracted with ethyl acetate (2×100 mL). The organic layers were combined washed with water (2×50 mL), brine (50 mL), dried and concentrated in vacuum. The residue obtained was purified by flash column chromatography [silica gel 12 g, eluting with ethyl acetate and methanol (9:1) in hexanes 0 to 50%] to afford 7-chloro-5-methyl-[1,2,4]triazolo[1,5-a]pyrimidine (55) (900 mg, 40.0%) as light yellow solid. ¹HNMR (300 MHz, DMSO) δ 8.76-8.61 (m, 1H), 7.64 (d, J=14.6, 1H), 2.63 (s, 3H); MS (ES⁺), 169.2 (M+1), 191.1 (M+Na); Analysis: Calcd for C₆H₅ClN₄: C, 42.74; H, 2.98; N, 33.23. Found: C, 42.83; H, 2.91; N, 33.25.

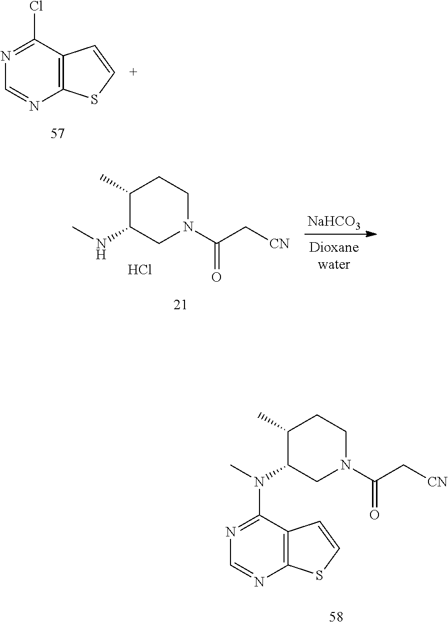

### Example 9

### 3-((3R,4R)-4-methyl-3-(methyl(thieno[2,3-d]pyrimidin-4-yl)amino)piperidin-1-yl)-3-oxopropanenitrile (58)

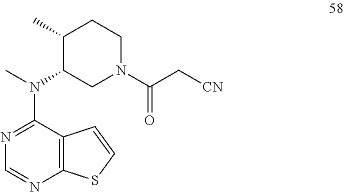

To a solution of 4-chlorothieno[2,3-d]pyrimidine (57) (0.1 g, 0.58 mmol) in dioxane (2 mL) was added 3-((3R,4R)-4-methyl-3-(methylamino)piperidin-1-yl)-3-oxopropanenitrile hydrochloride (21) (0.135 g, 0.58 mmol), sodium hydrogen carbonate (0.049 g, 0.58 mmol) and water (2.5 mL). The reaction mixture was heated in a microwave for 1 h (100° C., Power max on, Power 50 w). The reaction mixture was concentrated in vacuum and the residue obtained was purified by flash column chromatography (silica gel, 12 g, eluting with 0-50% CMA 80 in chloroform) to afford 3-((3R,4R)-4-methyl-3-(methyl(thieno[2,3-d]pyrimidin-4-yl)amino)piperidin-1-yl)-3-oxopropanenitrile (58) (0.017 g, 8.95%) as a white solid; mp 74.3° C. ¹HNMR (300 MHz, DMSO, 350K) δ 8.34 (s, 1H), 7.62 (d, J=6.2, 1H), 7.51 (d, J=6.2, 1H), 4.95 (dd, J=5.9, 12.0, 1H), 4.07-3.91 (m, 2H), 3.79 (s, 2H), 3.44 (s, 2H), 3.05 (s, 3H), 2.42 (s, 1H), 1.79 (s, 1H), 1.64 (s, 1H), 1.03 (d, J=7.1, 3H); MS (ES⁺) 330.1 (100%: M⁺¹).

Compound 57 is commercially available from Maybridge, or it can be prepared as described by, Hwang, Ki-Jun, et al., Archives of Pharmacal Research. 2001, 24(4), 270-275; Hesse, Stephanie, et al., Tetrahedron Letters, 2007, 48(30), 5261-5264; or Robba, Max, et al., Comptes Rendus des Seances de l'Academie des Sciences, Serie C: Sciences Chimiques, 1967, 264(2), 207-9.

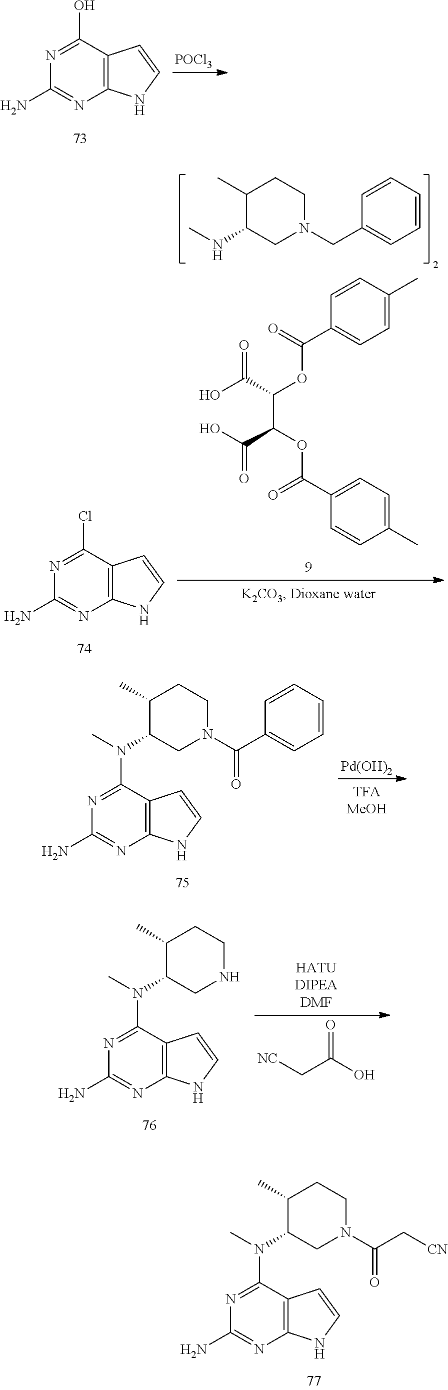

### Example 10

### 3-((3R,4R)-3-((2-amino-7H-pyrrolo[2,3-d]pyrimidin-4-yl)(methyl)amino)-4-methylpiperidin-1-yl)-3-oxopropanenitrile (77)

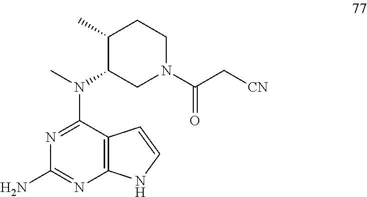

To a stirred solution of N-methyl-N-((3R,4R)-4-methylpiperidin-3-yl)-7H-pyrrolo[2,3-d]pyrimidin-4-amine trifluoroacetic acid salt (76) (0.82 g, 1.498 mmol) in dimethylformamide (10 mL) was added cyanoacetic acid (0.15 g, 1.79 mmol), diisopropylethyl amine (0.968 g, 7.49 mmol) and cooled to 0° C. To this mixture (2-(7-Aza-1H-benzotriazole-1-yl)-1,1,3,3-tetramethyluronium hexafluorophosphate) (HATU, 0.399 g, 1.051 mmol) was added and stirred at 20° C. for 18 h. The reaction mixture was quenched with water (10 mL), and concentrated in vacuum. The residue obtained was purified by flash column chromatography (silica gel 12 g, eluting with CMA 80 in chloroform 0 to 100%,), followed by another column chromatography [silica gel 12 g, eluting with a (9:1) ethyl acetate and methanol in hexanes 0 to 100%] to afford 3-((3R,4R)-3-((2-amino-7H-pyrrolo[2,3-d]pyrimidin-4-yl)(methyl)amino)-4-methylpiperidin-1-yl)-3-oxopropanenitrile (77) (165 mg, 33.6%) as a off-white solid. ¹HNMR (300 MHz, DMSO) δ 10.60 (s, 1H), 6.69 (d, J=3.4, 1H), 6.31 (d, J=3.5, 1H), 5.23 (s, 2H), 4.85-4.78 (m, 1H), 4.03-3.89 (m, 2H), 3.85-3.65 (m, 2H), 3.50-3.38 (m, 2H), 3.19 (s, 3H), 2.43-2.33 (m, 1H), 1.83-1.70 (m, 1H), 1.67-1.54 (m, 1H), 1.00 (d, J=7.1, 3H); MS (ES⁺): 328.1 (M+1), 350.1 (M+23); HPLC [Zorbax SBC3, 3.0×150 mm, 5 μm, with a ZGC SBC3, 2.1×12.5 mm guard cartridge, “A” Buffer=(98% of 0.1 M Ammonium Acetate in 2% acetonitrile) “B” Buffer=100% Acetonitrile, UV Absorbance; Rt=15.58 (97.32%)]

Preparation of Intermediate Compound (76)

a. To a mixture of 2,4-diamino-6-hydroxypyrimidine (50.0 g, 400 mmol) and sodium acetate (65.0 g, 792 mmol) in water (750 mL) heated at 65° C. was added a aqueous solution of chloroacetaldehyde (55 mL, 432 mmol, 50% in water). The reaction mixture was heated at 65° C. for 2 h and cooled to room temperature. The filtrate was decanted and concentrated in vacuum to 65% of the original volume and cooled in refrigerator overnight. The solid obtained was collected by filtration washed with water and dried in vacuum to furnish 2-amino-7H-pyrrolo[2,3-d]pyrimidin-4-ol (73) (52 g, contaminated by 15% NaOAc as seen from NMR for acetate peak). ¹H NMR (300 MHz, DMSO) δ 10.96 (s, 1H), 10.22 (s, 1H), 6.61 (dd, J=2.3, 3.4, 1H), 6.18 (dd, J=2.2, 3.4, 1H), 6.05 (s, 2H).
b. To a solution of 2-amino-7H-pyrrolo[2,3-d]pyrimidin-4-ol (73) (5.0 gm, 33 3 mmol from above step contaminated with sodium acetate 15%), dimethylaniline (4.22 mL, 41.0 mmol) and benzyltriethylammonium chloride (15.2 g, 66.6 mmol) in acetonitrile (25 mL) was added at room temperature POCl₃ (18.6 mL, 200 mmol). The reaction mixture was heated at reflux for 3 h and cooled to room temperature. The reaction mixture was concentrated in vacuum and the pH was adjusted to 5-6 using cold aqueous conc. NH₄OH solution. The reaction mixture was diluted with water (20 mL) and the solid obtained was collected by filtration dried in vacuum to furnish 4-chloro-7H-pyrrolo[2,3-d]pyrimidin-2-amine (74), which was pure enough to be taken to next step. ¹H NMR (300 MHz, DMSO) δ 11.46 (s, 1H), 7.09 (d, J=3.6, 1H), 6.49 (s, 2H), 6.25 (d, J=3.6, 1H).
c. A mixture of 4-chloro-7H-pyrrolo[2,3-d]pyrimidin-2-amine (74) (0.253 g, 1.5 mmol) bis[(1-benzyl-4-methylpiperidin-3-yl)-methylamine] di-p-toluoyl-L-tartarate (9) (0.74 g, 0.9 mmol) and K₂CO₃ (0.73 g, 5.25 mmol) in dioxane/water (1:1, 10 mL) was heated at reflux for 60 h. The reaction mixture was concentrated in vacuo and residue obtained was purified by flash column chromatography [silica gel, 24 g, eluting with 0-100% ethyl acetate/methanol (9:1) in hexane] to furnish N4-((3R,4R)-1-benzyl-4-methylpiperidin-3-yl)-N4-methyl-7H-pyrrolo[2,3-d]pyrimidine-2,4-diamine (75) (0.071 g, 14%) as a beige solid. ¹HNMR (300 MHz, DMSO) δ 10.68 (2s, 1H), 7.31 (d, J=4.4, 3H), 7.22 (dt, J=7.4, 14.6, 2H), 6.67 (dd, J=4.7, 7.1, 1H), 6.39 (2s, 1H), 5.37 (s, 2H), 5.01 (s, 1H), 3.56-3.37 (m, 4H), 2.73 (t, J=9.0, 1H), 2.60 (s, 1H), 2.27 (s, 1H), 2.09 (s, 1H), 1.69 (s, 1H), 1.60 (s, 1H), 0.88 (t, J=7.3, 3H); MS (ES+) 351.2 (M+1).
d. To a solution of N4-((3R,4R)-1-benzyl-4-methylpiperidin-3-yl)-N4-methyl-7H-pyrrolo[2,3-d]pyrimidine-2,4-diamine (75) (0.525 g, 1.5 mmol) in methanol (20 mL) was added trifluoroacetic acid (0.512 g, 4.49 mmol) and palladium hydroxide (0.55 g, 20 wt %, dry basis). The suspension was hydrogenated in a Parr shaker at 50 psi for 3.5 h. The reaction mixture was diluted with methanol (50 mL) and filtered through Celite to remove catalyst. The filtrate was concentrated in vacuum to furnish trifluoroacetic acid salt of N-methyl-N-((3R,4R)-4-methylpiperidin-3-yl)-7H-pyrrolo[2,3-d]pyrimidin-4-amine (76). MS (ES⁺): 261.1 (M+1).

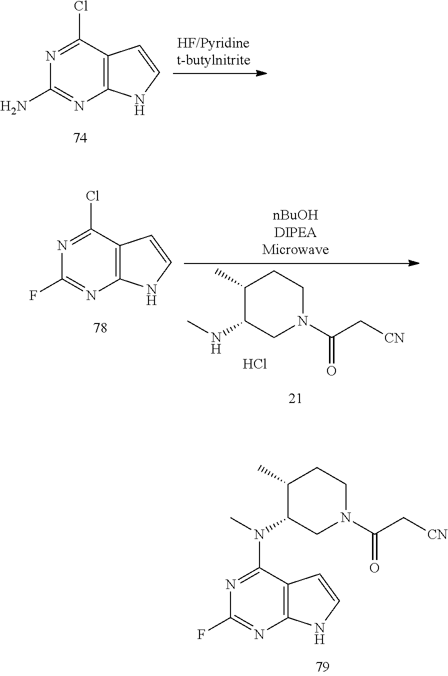

### Example 11

### 3-((3R,4R)-3-((2-Fluoro-7H-pyrrolo[2,3-d]pyrimidin-4-yl)(methyl)amino)-4-methylpiperidin-1-yl)-3-oxopropanenitrile (79)

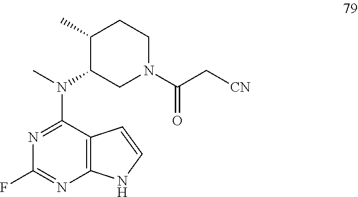

A mixture of 4-Chloro-2-fluoro-7H-pyrrolo[2,3-d]pyrimidine (78) (0.117 g, 0.68 mmol) 3-((3R,4R)-4-methyl-3-(methylamino)piperidin-1-yl)-3-oxopropanenitrile (21) (0.189 g, 0.82 mmol) and DIPEA (0.475 mL, 2.72 mmol) in n-butanol (2 mL) was heated in a microwave at 125° C. for 3 h. The reaction mixture was concentrated in vacuo and purified by flash column chromatography [silica gel 24 g, eluting with 0-100% ethyl acetate/methanol (9:1) in hexanes] to furnish 3-((3R,4R)-3-((2-Fluoro-7H-pyrrolo[2,3-d]pyrimidin-4-yl)(methyl)amino)-4-methylpiperidin-1-yl)-3-oxopropanenitrile (79) (0.02 g, 9%) as a off-white solid. ¹HNMR (300 MHz, DMSO) δ 11.80 (s, 1H), 7.12 (s, 1H), 6.60 (s, 1H), 4.70 (s, 1H), 4.12 (d, J=5.9, 2H), 3.96-3.59 (m, 2H), 3.38 (d, J=11.0, 2H), 3.26 (s, 3H), 2.39 (s, 1H), 1.82 (s, 1H), 1.59 (s, 1H), 1.01 (d, J=7.1, 3H); ¹⁹FNMR (300 MHz, DMSO) δ −54.03; HPLC [Zorbax SBC3, 3.0×150 mm, 5 μm, with a ZGC SBC3, 2.1×12.5 mm guard cartridge, “A” Buffer=(98% of 0.1 M Ammonium Acetate in 2% acetonitrile) “B” Buffer=100% Acetonitrile, UV Absorbance; Rt=16.10 (98.29%)].

Preparation of Intermediate Compound (78)

a. To a solution of 4-chloro-7H-pyrrolo[2,3-d]pyrimidin-2-amine (74) (0.464 g, 2.75 mmol) in HF in pyridine (10 mL, 70% HF in 30% pyridine) in a Teflon bottle cooled to −60° C. was added dropwise t-butyl nitrite (0.98 mL, 8.25 mmol). The reaction was allowed to warm to −40° C. over a 2-h period and diluted with chloroform (100 mL). The reaction mixture was carefully poured into ice cold solution of water containing K₂CO₃ (3 g). The reaction mixture was neutralized with saturated aqueous solution of NaHCO₃. The organic layer was separated, washed with brine (25 mL), dried, filtered and concentrated in vacuo. The residue obtained was purified by flash column chromatography (silica gel, 24 g, eluting with 0-100% ethyl acetate in hexane) to furnish 4-Chloro-2-fluoro-7H-pyrrolo[2,3-d]pyrimidine (78) (0.25 g, 53%) as an off-white solid; mp 180.0° C.; ¹H NMR (300 MHz, DMSO) δ 12.72 (s, 1H), 7.68 (d, J=3.6, 1H), 6.67 (d, J=3.6, 1H); ¹⁹F NMR (300 MHz, DMSO) δ −54.77. MS: Analysis: Calcd for C₆H₃ClFN₃: C, 42.01; H, 1.76; N, 24.49; Cl, 20.67. Found: C, 42.23; H, 1.70; N, 24.58; Cl, 20.40.

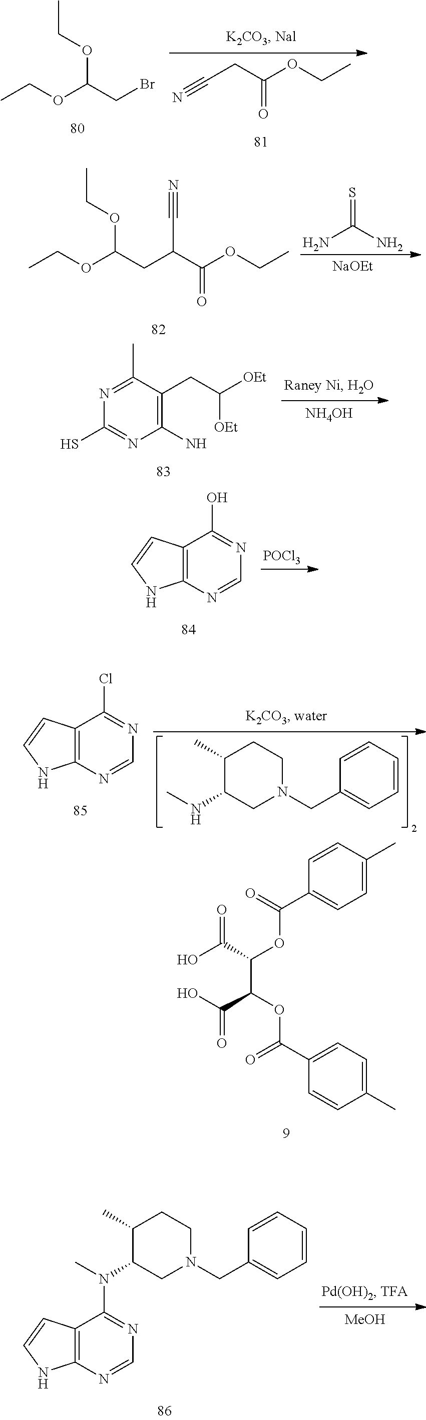

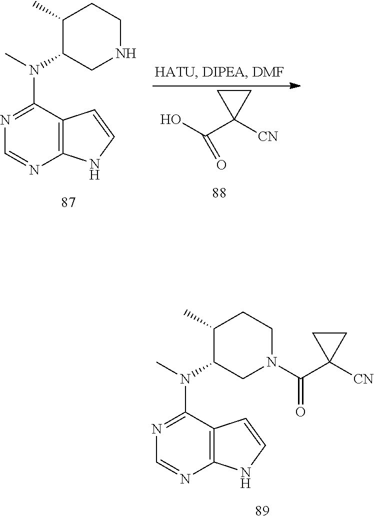

### Example 12

### 1-((3R,4R)-4-Methyl-3-(methyl(7H-pyrrolo[2,3-d]pyrimidin-4-yl)amino)piperidine-1-carbonyl)cyclopropanecarbonitrile (89)

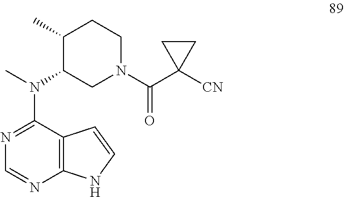

To a stirred solution of N-methyl-N-((3R,4R)-4-methylpiperidin-3-yl)-7H-pyrrolo[2,3-d]pyrimidin-4-amine (87) (0.129 g, 0.52 mmol) in Dimethylformamide (1 mL) was added 1-cyanocyclopropanecarboxylic acid (88) (0.089 g, 1.051 mmol), diisopropylethylamine (0.27 g, 2.10 mmol) and cooled to −10° C. To this mixture (2-(7-Aza-1H-benzotriazole-1-yl)-1,1,3,3-tetramethyluronium hexafluorophosphate) (HATU, 0.399 g, 1.051 mmol) was added and stirred at 10° C. for 1.5 h. The reaction mixture was quenched with water (10 mL), extracted with a (9:1) mixture of ethyl acetate and methanol (3×50 mL). The organic layers were combined washed with water (2×15 mL), brine (10 mL), dried and concentrated in vacuum. The residue obtained was purified by flash column chromatography (silica gel 12 g, eluting with CMA 80 in chloroform 0 to 100%) to afford 1-((3R,4R)-4-Methyl-3-(methyl(7H-pyrrolo[2,3-d]pyrimidin-4-yl)amino)piperidine-1-carbonyl)cyclopropanecarbonitrile (89) (100 mg, 56.82%) as a light beige solid. ¹HNMR (300 MHz, DMSO) δ 11.66 (s, 1H), 8.10 (s, 1H), 7.18-7.09 (m, 1H), 6.58 (s, 1H), 4.94 (s, 1H), 4.37-3.63 (m, 4H), 3.33 (s, 3H), 2.47-2.35 (m, 1H), 1.93-1.79 (m, 1H), 1.84-1.45 (m, 5H), 1.04 (d, J=7.1, 3H); MS (ES⁺): 339.1 (M+1); HPLC [Zorbax SBC3, 3.0×150 mm, 5 um, with a ZGC SBC3, 2.1×12.5 mm guard cartridge, “A” Buffer=(98% of 0.1 M Ammonium Acetate in 2% acetonitrile) “B” Buffer=100% Acetonitrile, UV Absorbance; Rt=16.65 (97.71%)]; Analysis: Calcd for C₁₈H₂₂N₆O.0.25 H₂O: C, 63.04; H, 6.61; N, 24.50. Found: C, 63.40; H, 6.54; N, 24.28.

Preparation of Intermediate Compound (87)

a. A mixture of ethyl cyanoacetate 81 (227.97 g, 2015.52 mmol), bromo acetaldehyde diethyl ether (80) (80 g, 405.94 mmol), potassium carbonate (55.99 g, 405.13 mmol) and sodium iodide (4 g, 26.67 mmol) was refluxed for 20 h (CO₂ evolution was observed during the reaction). The reaction mixture was stirred at reflux for additional 4 h after the evolution of CO₂ has ceased. The reaction was cooled to room temperature, diluted with water (400 mL) and diethyl ether (400 mL). The organic layer was separated and the aqueous layer was extracted with diethyl ether (250 mL). The ether layers were combined washed with water (2×100 mL), brine (200 mL), dried, filtered and concentrated in vacuum. The product obtained was distilled under vacuum to furnish ethyl-2-cyano-4,4-diethoxybutanoate (82) (47.5 g, 51.0%) as a colorless oil; B.P: 103° C./1 mm Hg. ¹HNMR (300 MHz, DMSO) δ 4.61 (t, J=5.7, 1H), 4.24-4.08 (m, 3H), 3.67-3.54 (m, 2H), 3.53-3.40 (m, 2H), 2.12 (t, J=6.0, 2H), 1.23 (t, J=7.1, 3H), 1.11 (td, J=4.9, 7.0, 6H); IR (neat): 3482, 2980, 2901, 2361, 2252, 1749, 1446, 1374, 1262, 1218, 1128, 1062 and 857 cm⁻¹; MS (ES⁻): 263.6 (M+35); Analysis: Calc for C₁₁H₁₉NO₄.0.25 H₂O: C, 56.51; H, 8.40; N, 5.99. Found: C, 56.71; H, 8.16; N, 5.96.
b. To a freshly prepared solution of sodium ethoxide [ethanol (250 mL) and sodium metal (9.02 g, 392.55 mmol)] was added ethyl 2-cyano-4,4-diethoxybutanoate (82) (45 g, 196.27 mmol) and thiourea (14.94 g, 196.27 mmol) in ethanol (200 mL). The reaction mixture was heated with stirring at reflux for 3.5 h. The reaction mixture was allowed to cool to room temperature and stirred overnight. The reaction was quenched with water (100 mL) and concentrated in vacuum to remove ethanol. The residue obtained was dissolved in water (100 mL) and neutralized to pH 7 using dilute aqueous hydrochloric acid (3N) by maintaining the temperature below 10° C. The solid obtained was collected by filtration to afford on drying in vacuum 6-amino-5-(2,2-diethoxyethyl)-2-mercaptopyrimidin-4-ol (83) (30.6 g, 60.19%) as a pale yellow solid. ¹H NMR (300 MHz, DMSO) 11.75 (s, 1H, D₂O exchangeable), 11.44 (s, 1H, D₂O exchangeable), 6.07 (s, 2H, D₂O exchangeable), 4.50 (t, J=5.6, 1H), 3.59 (dq, J=7.0, 9.5, 2H), 3.40 (dq, J=7.0, 9.6, 2H), 2.44 (d, J=5.6, 2H), 1.07 (t, J=7.0, 6H); IR (KBr): 3226, 2973, 2909, 1624, 1569, 1474, 1376, 1287, 1213, 1114, 1049, 993, 892, 822, 789 and 763 cm⁻¹; MS (ES⁺¹) 260.1 (M+1), 282.1 (M+23), (ES⁻): 258.3 (M−1); HPLC [(Column: Zorbax SBC3, 3.0×150 mm, 5 with a ZGC SBC3, 2.1×12 5 mm guard cartridge. Mobile phase: 0.1 M Ammonium Acetate/Acetonitrile) Rt=11.408 min (99.64%]); Analysis: Calculated for C₁₀H₁₇N₃O₃S: C, 46.45; H, 6.72; N, 16.06. Found: C, 46.31; H, 6.60; N, 16.20.
c. To the slurry of 6-amino-5-(2,2-diethoxyethyl)-2-mercaptopyrimidin-4-ol (83) (29 g, 111.96 mmol) and Raney Ni (87 g) in water (1000 mL) was added conc. aqueous ammonium hydroxide (90 mL) at room temperature. The reaction mixture was heated at reflux for 1 h and filtered through celite to remove catalyst. The filtrate was concentrated to 770 mL and neutralized with conc. Hydrochloric acid (13 mL). The reaction was stirred for 16 h and the solid obtained was collected by filtration to afford on drying in vacuum 7H-pyrrolo[2,3-d]pyrimidin-4-ol (84) (12.6 g, 83.3%) as a colorless solid. ¹HNMR (300 MHz, DMSO) δ 11.85 (s, 1H, D₂O exchangeable), 11.77 (s, 1H, D₂O exchangeable), 7.82 (s, 1H), 7.08-6.98 (m, 1H), 6.43 (dd, J=2.1, 3.3, 1H); MS (ES⁺¹) 136.2 (M+1), 158.2 (M+23); HPLC [Column: Zorbax SBC3, 3.0×150 mm, 5 gm, with a ZGC SBC3, 2.1×12.5 mm guard cartridge. Mobile phase: 0.1 M Ammonium Acetate/Acetonitrile) Rt=5.214 min (100%)]; Analysis: Calculated for C₆H₅N₃O: C, 53.33; H, 3.72; N, 31.09. Found: C, 52.97; H, 3.66; N, 30.77.
d. A suspension of 7H-pyrrolo[2,3-d]pyrimidin-4-ol (84) (5 g, 37.00 mmol) in Phosphorous oxy chloride (50 mL) was heated at reflux with stirring for 1.5 h. The reaction mixture was cooled and concentrated in vacuum to remove Phosphorous oxy chloride. To the residue obtained was added ice cold water and stirred for 30 min. The reaction mixture was extracted with diethyl ether (2×500 mL). The organic layers were combined, washed with water (2×200 mL); brine (100 mL) dried and concentrated in vacuum. The residue was triturated with hexanes, and the solid obtained was collected by filtration to afford on drying in vacuum 4-chloro-7H-pyrrolo[2,3-d]pyrimidine (85) (2.467 g, 43.4%) as a white crystalline solid. ¹HNMR (300 MHz, DMSO) δ 12.58 (s, 1H, D₂O exchangeable), 8.60 (s, 1H), 7.70 (d, J=3.5, 1H), 6.61 (d, J=3.5, 1H); HPLC [Column: Zorbax SBC3, 3.0×150 mm, 5 μm, with a ZGC SBC3, 2.1×12.5 mm guard cartridge. Mobile phase: 0.1 M Ammonium Acetate/Acetonitrile) Rt=12.76 min. (97.97%).
e. A stirred suspension of bis[(1-benzyl-4-methylpiperidin-3-yl)-methylamine] di-p-toluoyl-L-tartarate (9) (0.685 g, 0.83 mmol), 4-chloro-7H-pyrrolo[2,3-d]pyrimidine (85) (0.24 g, 1.60 mmol) and potassium carbonate (0.66 g, 4.80 mmol) in water (5 mL) was heated at 100° C. for 108 h. The reaction mixture was cooled to room temperature, diluted with water (10 mL), Toluene (100 mL) and filtered. The toluene layer was washed with aqueous 1 N sodium hydroxide solution (2×20 mL), water (2×20 mL), brine (20 mL), dried, filtered and concentrated in vacuo. The crude residue obtained was purified by flash chromatography [silica gel 12 g, eluting with ethyl acetate/methanol (9:1) in hexanes 0 to 100%] to afford N-((3R,4R)-1-Benzyl-4-methylpiperidin-3-yl)-N-methyl-7H-pyrrolo[2,3-d]pyrimidin-4-amine (86) (0.237 g, 44.1%) as an off-white solid. ¹HNMR (300 MHz, DMSO) δ 11.59 (s, 1H), 8.06 (s, 1H), 7.35-7.19 (m, 5H), 7.12-7.08 (m, 1H), 6.55 (s, 1H), 5.10 (s, 1H), 3.57-3.43 (m, 5H), 2.78 (dd, J=6.3, 11.5, 1H), 2.68-2.53 (m, 2H), 2.35-2.24 (m, 1H), 2.19-2.04 (m, 1H), 1.66 (d, J=23.6, 2H), 0.89 (d, J=7.0, 3H); MS (ES⁺): 336.2 (M+1).
f. To a solution of N-((3R,4R)-1-benzyl-4-methylpiperidin-3-yl)-N-methyl-7H-pyrrolo[2,3-d]pyrimidin-4-amine (86) (0.16 g, 0.47 mmol) in methanol (10 mL) was added trifluoroacetic acid (0.108 g, 0.95 mmol) and palladium hydroxide (0.16 g, 20 wt %,). The suspension was hydrogenated in a Parr shaker at 50 psi for 5.5 h. The reaction mixture was diluted with methanol (50 mL), filtered through a pad of Celite to remove catalyst and the filtrate was concentrated in vacuum. The crude residue obtained was purified by flash column chromatography (silica gel, eluting with 0-25% CMA 80 in chloroform) to furnish N-Methyl-N-((3R,4R)-4-methylpiperidin-3-yl)-7H-pyrrolo[2,3-d]pyrimidin-4-amine (87) (0.45 g, 39%) as a colorless solid; mp 158.4° C. ¹H NMR (300 MHz, DMSO) δ 11.59 (s, 1H, D₂O exchangeable), 8.08 (d, J=5.6 Hz, 1H), 7.12 (d, J=1.6 Hz, 1H), 6.54 (d, J=3.0 Hz, 1H), 4.79 (s, 1H), 3.32 (s, 4H, CH₃, NH, D₂O, exchangeable), 3.13 (dd, J=9.1, 12.0 Hz, 1H), 2.88-2.71 (m, 2H), 2.63 (dt, J=4.2, 12.4 Hz, 1H), 2.37-2.26 (m, 1H), 1.74 (ddd, J=4.4, 9.5, 14.5 Hz, 1H), 1.54-1.42 (m, 1H), 0.98 (d, J=7.2 Hz, 3H); MS (ES⁺): 246.1 (M+1); Analysis: Calculated for C₁₃H₁₉N₅: C, 63.64; H, 7.80; N, 28.54. Found: C, 63.95; H, 7.83; N, 28.20.

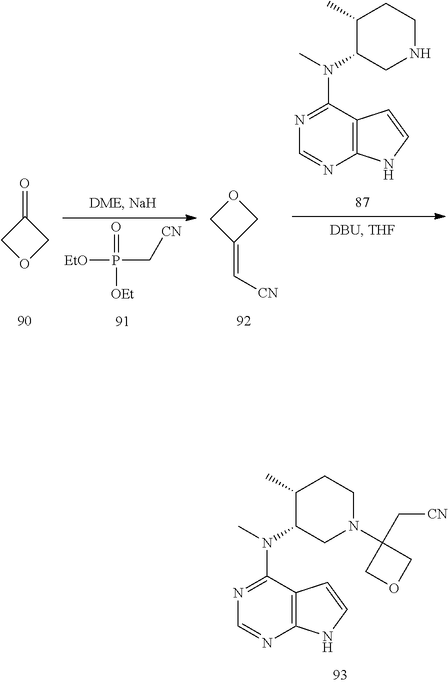

### Example 13

### 2-(3-((3R,4R)-4-Methyl-3-(methyl(7H-pyrrolo[2,3-d]pyrimidin-4-yl)amino)piperidin-1-yl)oxetan-3-yl)acetonitrile (93)

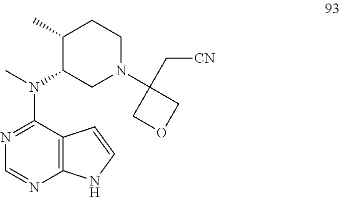

To a stirred solution of N-methyl-N-((3R,4R)-4-methylpiperidin-3-yl)-7H-pyrrolo[2,3-d]pyrimidin-4-amine (87) (0.1 g, 0.407 mmol) in tetrahydrofuran (10 mL) was added 2-(oxetan-3-ylidene)acetonitrile (92) (0.038, 0.407 mmol) and 1,4-Diazabicyclo[5.4.0]undec-7-ene (0.062 g, 0.407 mmol) at 20° C. The reaction mixture was heated at reflux for 18 h, cooled to room temperature and quenched with water (5 mL). The reaction mixture was extracted with ethyl acetate (2×50 mL). The organic layers were combined washed with water (2×20 mL), brine (2×20 mL), dried, filtered and concentrated in vacuum. The crude residue obtained was purified twice by flash chromatography [silica gel 12 g, eluting with ethyl CMA 80 in chloroform 0 to 20%, second time eluting with ethyl acetate/methanol (9:1) in hexanes 0 to 100%] to afford 2-(3-((3R,4R)-4-Methyl-3-(methyl(7H-pyrrolo[2,3-d]pyrimidin-4-yl)amino)piperidin-1-yl)oxetan-3-yl)acetonitrile (93) (0.004 g, 2.88%) as an off-white solid. ¹HNMR (300 MHz, MeOD) δ 8.08 (s, 1H), 7.09 (d, J=3.6, 1H), 6.66 (d, J=3.6, 1H), 5.15 (d, J=3.9, 1H), 4.65 (t, J=6.2, 2H), 4.50 (dd, J=6.4, 10.3, 2H), 3.65 (s, 3H), 3.00 (s, 2H), 2.91 (dd, J=6.0, 11.3, 1H), 2.71 (dd, J=3.7, 11.3, 2H), 2.48-2.35 (m, 1H), 2.23 (s, 1H), 1.92-1.68 (m, 2H), 0.99 (d, J=7.1, 3H); MS (ES⁺): 341.1 (M+1), 363.1 (M+23). HPLC [Zorbax SBC3, 3.0×150 mm, 5 μm, with a ZGC SBC3, 2.1×12.5 mm guard cartridge, “A” Buffer=(98% of 0.1 M Ammonium Acetate in 2% acetonitrile) “B” Buffer=100% Acetonitrile, UV Absorbance; Rt=16.75 (100%)].

Preparation of Intermediate Compound (92)

a. To slurry of sodium hydride (4.12 g, 102.83 mmol) in DME (120 mL) at 0-5° C. was added diethylcyanomethyl phosphonate (91) (16.2 mL, 99.8 mmol) at a rate maintaining reaction temperature at 5° C. The heterogeneous mixture became homogenous after stirring for 30 mins at 0-5° C. To this mixture was added, a solution of oxetan-3-one (90) (10.1 g, 83 2 mmol) in DME (20 mL) dropwise at 5° C. and the mixture was allowed to warm to room temperature overnight. The reaction was quenched with water (250 mL) and extracted with ethyl acetate (200 mL, 100 mL). The organic layers were combined and washed with brine (200 mL), dried over MgSO₄, filtered and the filtrate was concentrated in vacuum to dryness to furnish 2-(Oxetan-3-ylidene)acetonitrile (92) (8.0 g, 60%) as an oil, which solidifies on standing. ¹HNMR (300 MHz, DMSO-d₆): δ 5.43-5.35 (m, 2H), 5.35-5.23 (m, 3H); ¹³C NMR (300 MHz, DMSO) δ 163.57, 114.17, 90.88, 78.66, 78.53. IR (KBr) 2219 cm⁻¹; Analysis: Calculated for C₅H₅NO: C, 63.15; H, 5.30; N, 14.73. Found: C, 63.00; H, 5.36; N, 14.44.

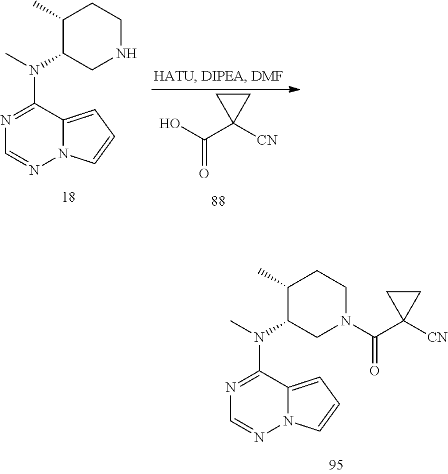

### Example 14

### 1-((3R,4R)-4-methyl-3-(methyl(pyrrolo[1,2-f][1,2,4]triazin-4-yl)amino)piperidine-1-carbonyl)cyclopropanecarbonitrile (95)

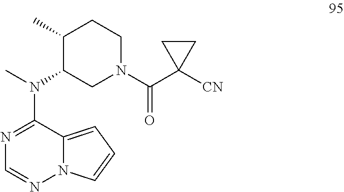

To a solution of N-methyl-N-((3R,4R)-4-methylpiperidin-3-yl)pyrrolo[1,2-f][1,2,4]triazin-4-amine (18) (0.20 g, 0.81 mmol) in Dimethylformamide (5 mL) was added 1-cyanocyclopropanecarboxylic acid (88) (0.099 g, 0.89 mmol), diisopropylethyl amine (0.26 g, 2.03 mmol) and cooled to −10° C. To this mixture (2-(7-Aza-1H-benzotriazole-1-yl)-1,1,3,3-tetramethyluronium hexafluorophosphate) (HATU, 0.34 g, 0.89 mmol) was added and stirred below 10° C. for 1 h. The reaction mixture was quenched with water (15 mL) and extracted with of ethyl acetate (3×50 mL). The organic layers were combined washed with water (2×15 mL), brine (10 mL), dried and concentrated in vacuo. The residue obtained was purified by flash column chromatography (silica gel 12 g, eluting with CMA 80 in chloroform 0 to 100%) to afford 1-((3R,4R)-4-methyl-3-(methyl(pyrrolo[1,2-f][1,2,4]triazin-4-yl)amino)piperidine-1-carbonyl)cyclopropanecarbonitrile (95) (125 mg, 45.6%) as a light beige solid; ¹H NMR (300 MHz, DMSO) δ 7.83 (s, 1H), 7.72 (dd, J=1.5, 2.6, 1H), 6.95 (d, J=3.9, 1H), 6.68 (dd, J=2.7, 4.6, 1H), 4.99 (s, 1H), 4.03-3.70 (m, 4H), 3.40 (s, 3H), 2.49-2.38 (m, 1H), 1.94-1.76 (m, 1H), 1.75-1.59 (m, 3H), 1.56-1.45 (m, 2H), 1.07 (d, J=7.2, 3H); MS (ES⁺): 339.1 (M+1); HPLC (Zorbax SBC3, 3.0×150 mm, 5 μm, with a ZGC SBC3, 2.1×12.5 mm guard cartridge, “A” Buffer=(98% of 0.1 M Ammonium Acetate in 2% acetonitrile) “B” Buffer=100% Acetonitrile, UV Absorbance; Rt==17.207 (97.84%)); Analysis; Calculated for C₁₈H₂₂N₆O.0.5 H₂O: C, 62.22; H, 6.67; N, 24.19. Found: C, 62.07; H, 6.85; N, 24.00.

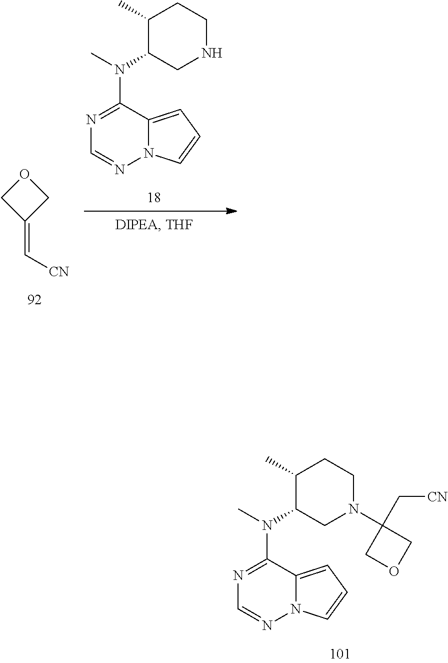

### Example 15

### 2-(3-((3R,4R)-4-methyl-3-(methyl(pyrrolo[1,2-f][1,2,4]-triazin-4-yl)amino)piperidin-1-yl)oxetan-3-yl)acetonitrile (101)

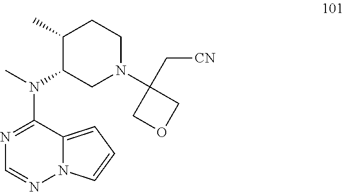

To a solution of N-methyl-N-((3R,4R)-4-methylpiperidin-3-yl)pyrrolo[1,2-f][1,2,4]triazin-4-amine (18) (0.30 g, 1.22 mmol) in THF (20 mL) was added 2-(oxetan-3-ylidene)acetonitrile (92) (0.127, 1.34 mmol), and diisopropylethyl amine (0.43 mL, 2.44 mmol) and stirred at room temperature for 48 h. The reaction mixture was concentrated in vacuo. The residue obtained was purified by flash column chromatography (silica gel 12 g, eluting with CMA 80 in chloroform 0 to 100%) to afford 2-(3-((3R,4R)-4-methyl-3-(methyl(pyrrolo[1,2-f][1,2,4]triazin-4-yl)amino)piperidin-1-yl)oxetan-3-yl)acetonitrile (101) (10 mg, 3%) as a off white solid; ¹H NMR (300 MHz, CDCl₃) δ 7.81 (s, 1H), 7.59 (dd, J=1.5, 2.6, 1H), 6.81 (d, J=3.7, 1H), 6.64 (dd, J=2.7, 4.6, 1H), 5.31 (s, 1H), 4.65 (dd, J=6.3, 15.8, 2H), 4.42 (dd, J=6.3, 25.7, 2H), 3.79 (s, 3H), 2.91-2.82 (m, 3H), 2.79-2.69 (m, 2H), 2.47-2.37 (m, 1H), 2.27-2.12 (M, 1H), 1.87-1.71 (m, 2H), 1.00 (d, J=7.0, 3H); IR (KBr) 2243 cm⁻¹; MS (ES⁻): 375.0 (M+35); HPLC [Modified 5191 method, Zorbax SBC3, 3.0×150 mm, 5 μm, with a ZGC SBC3, 2.1×12.5 mm guard cartridge, “A”Buffer=(98% of 0.1 M Ammonium Acetate in 2% acetonitrile) “B” Buffer=100% Acetonitrile, UV Absorbance; Rₜ=17.361 (95.62%)].

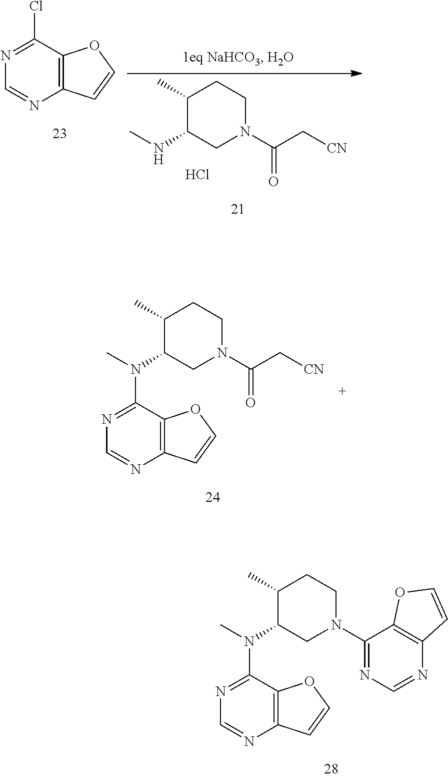

### Example 16

### N-((3R,4R)-1-(Furo[3,2-d]pyrimidin-4-yl)-4-methylpiperidin-3-yl)-N-methylfuro[3,2-d]pyrimidin-4-amine (28)

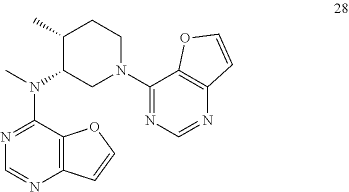

To a solution of 4-chlorofuro[3,2-d]pyrimidine (23) (0.233 g, 1.5 mmol) in dioxane (2 mL) was added 3-((3R,4R)-4-methyl-3-(methylamino)piperidin-1-yl)-3-oxopropanenitrile hydrochloride (21) (0.349 g, 1.5 mmol), sodium bicarbonate (126 mg, 1.5 mmol) and water (5 mL). The reaction mixture was heated with stirring at reflux for 1 h, cooled to room temperature, diluted with water (10 mL), and extracted with ethyl acetate (2×100 mL). The organic layers were combined, washed with water (20 mL), brine (10 mL), dried and concentrated in vacuum. The residue obtained was purified by flash column chromatography (silica gel 12 g, eluting with 0-50% CMA 80 in chloroform) to furnish N-((3R,4R)-1-(Furo[3,2-d]pyrimidin-4-yl)-4-methylpiperidin-3-yl)-N-methylfuro[3,2-d]pyrimidin-4-amine (28) (7 mg, 1.3%) as a white solid. ¹HNMR (300 MHz, DMSO) δ 8.33 (s, 2H), 8.00 (d, J=6.2, 2H), 6.86 (s, 2H), 5.18 (s, 1H), 4.56-4.49 (m, 1H), 4.44-4.29 (m, 2H), 4.20-4. (s, 1H), 3.42 (s, 3H), 2.59-2.46 (m, 1H), 2.04-1.94 (m, 1H), 1.92-1.78 (s, 1H), 1.16 (d, J=7.0, 3H). MS (ES⁺) 365.1 (100%: M⁺¹), 387 (50%, M+23). Further elution gave 3-((3R,4R)-3-(furo[3,2-d]pyrimidin-4-yl(methyl)amino)-4-methylpiperidin-1-yl)-3-oxopropanenitrile (24) (34 mg, 7.23%) as a white solid; mp 107.7° C. ¹H NMR (300 MHz, DMSO) (350° K) δ 8.34 (s, 1H), 8.16 (d, J=2.2, 1H), 6.92 (d, J=2.1, 1H), 4.87 (dd, J=12.0, 6.9 Hz, 1H), 4.09-3.89 (m, 2H), 3.82 (s, 2H), 3.45 (s, 2H), 3.31 (s, 3H), 2.37 (s, 1H), 1.85-1.58 (m, 2H), 1.01 (d, J=7.1 Hz, 3H); MS (ES⁻) 3.4.1 (100%: M⁻¹), 336 (30%, M+23).

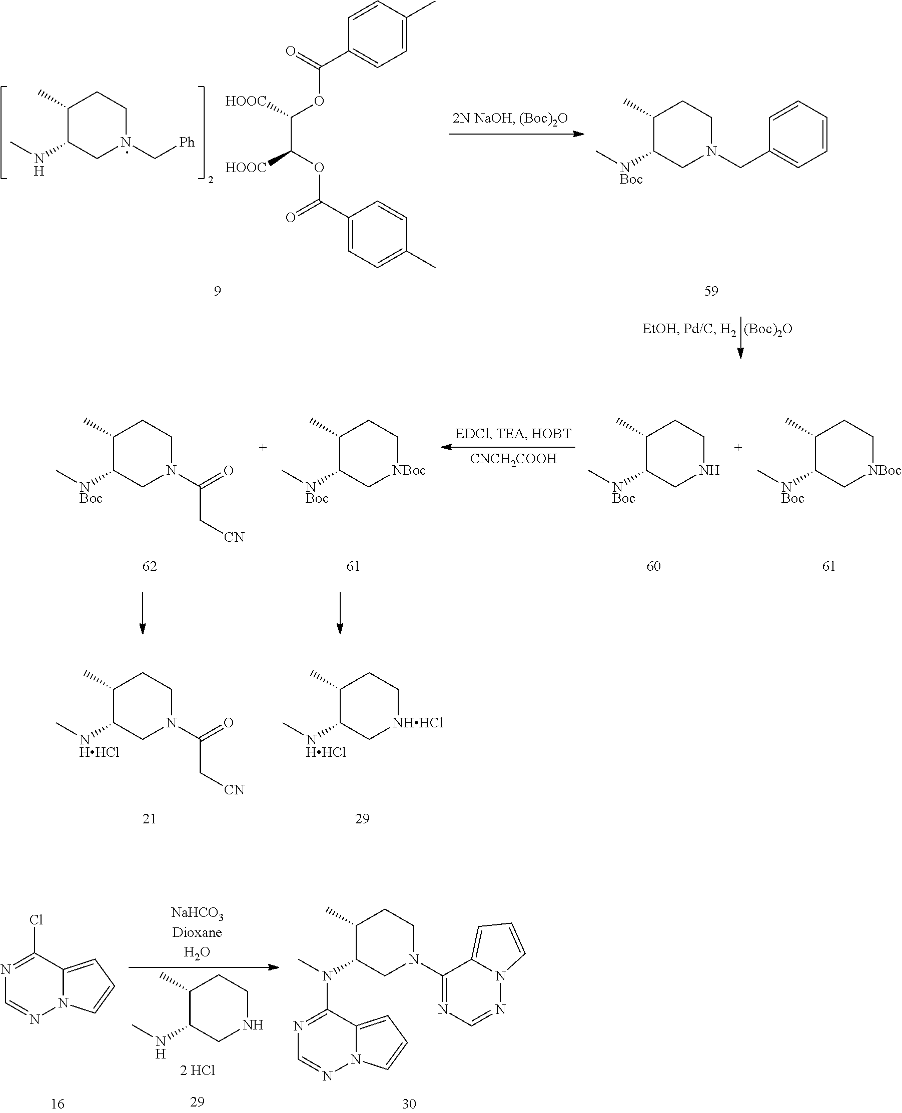

### Example 17

### N-Methyl-N-((3R,4R)-4-methyl-1-(pyrrolo[1,2-f][1,2,4]triazin-4-yl)piperidin-3-yl)pyrrolo[1,2-f][1,2,4]triazin-4-amine (30)

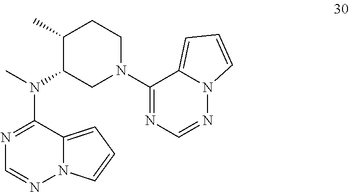

A mixture of (3R,4R)—N,4-dimethylpiperidin-3-amine dihydrochloride (29) (0.1 g, 0.49 mmol), 4-chloropyrrolo[1,2-f][1,2,4]triazine (16) (0.16 g, 1.04 mmol), sodium hydrogen carbonate (0.093 g, 1.11 mmol) in dioxane (2 mL) and water (2 mL) was subjected to microwave irradiation at 100° C., for 10 minutes. Additional 4-chloropyrrolo[1,2-f][1,2,4]triazine (0.05 g, 0.32 mmol) and sodium hydrogen carbonate (0.05 g, 0.59 mmol) were added and continued microwave heating at 100° C. for 50 min. The reaction mixture was concentrated in vacuum and the residue obtained was purified by flash column chromatography (silica gel 12 g, eluting with ethyl acetate in hexanes 0 to 100%) to furnish N-Methyl-N-((3R,4R)-4-methyl-1-(pyrrolo[1,2-f][1,2,4]triazin-4-yl)piperidin-3-yl)pyrrolo[1,2-f][1,2,4]triazin-4-amine (30) (0.12 g, 67.5%) as a white solid; mp 103.4° C. ¹HNMR (300 MHz, DMSO)S 7.87 (s, 1H), 7.80 (s, 1H), 7.72 (td, J=1.4, 2.9, 2H), 6.96 (dd, J=1.3, 4.6, 2H), 6.67 (td, J=2.7, 4.5, 2H), 5.11 (s, 1H), 4.41 (dd, J=3.8, 13.1, 1H), 4.29-4.10 (m, 2H), 4.02-3.88 (m, 1H), 3.41 (s, 3H), 1.87 (dd, J=4.4, 8.9, 1H), 1.81-1.64 (m, 2H), 1.11 (d, J=7.1, 3H); MS (ES+) 363.1 (100%: M⁺¹). HPLC [(Zorbax SBC3, 3.0×150 mm, 5 μm, with a ZGC SBC3, 2.1×12.5 mm guard cartridge. mobile phase: 0.1 M ammonium acetate/acetonitrile) Rt=19.482 min, (98.92%)]; Analysis: Calcd for C₁₉H₂₂N₈.0.25 H₂O: C, 62.19; H, 6.18; N, 30.53. Found: C, 62.11; H, 6.01; N, 30.14.

Preparation of Intermediate Compound (29)

a. To a solution of di-((3R,4R)-1-benzyl-N,4-dimethylpiperidin-3-amine) di-p-toluoyl-L-tartarate (9) (20.0 g, 48 mmol) in dioxane/water (2:1, 120 mL) was added 3 N NaOH (25.6 mL, 76.8 mmol)) and boc anhydride (11.52 g, 52.8 mmol). The reaction was stirred at room temperature overnight. TLC analysis showed no reaction (pH was not basic). To the reaction mixture was added 3N NaOH (16 mL, 48 mmol), boc anhydride (10.5 g, 48 mmol) and stirred at room temperature for 2 h. The reaction mixture was concentrated in vacuo to remove dioxane diluted with water (50 mL) and extracted twice with ethyl acetate (150 mL). The organic layers were combined, washed with brine (100 mL), dried over MgSO₄ and filtered. The filtrate was concentrated in vacuo and the residue obtained was purified by flash column chromatography (silica gel, 240 g eluting with ethyl acetate in hexanes 0-40%) to furnish tert-butyl (3R,4R)-1-benzyl-4-methylpiperidin-3-yl(methyl)carbamate (59) (17.9 g, 82%) as a colorless oil, which was contaminated with boc anhydride (From NMR analysis). This was used as such for next step. ¹H NMR (300 MHz, DMSO) δ 7.47-7.18 (m, 5H), 4.03 (d, J=7.1 Hz, 1H), 3.42 (q, J=13.1 Hz, 2H), 3.01 (s, 3H), 2.66 (m, 2H), 2.36 (m, 1H), 2.12 (m, 1H), 1.86 (m, 1H), 1.51 (m, 2H), 1.37 (s, 9H), 0.86 (d, J=7.0 Hz, 3H); MS (ES⁺): 319.2 (100%, M⁺¹).
b. To a solution of above tert-butyl (3R,4R)-1-benzyl-4-methylpiperidin-3-yl(methyl)carbamate (59) (17.9 g) in ethanol (200 mL) was added Pd/C (10% on carbon, 1.5 g) and hydrogenated on a Parr Shaker at 60 psi for 72 h. The reaction mixture was filtered through a pad of Celite and the filtrate was concentrated in vacuo to furnish a mixture of tert-butyl methyl ((3R,4R)-4-methylpiperidin-3-yl)carbamate (60) and (3R,4R)-tert-butyl 3-(tert-butoxycarbonyl(methyl)amino)-4-methylpiperidine-1-carboxylate (61) (12.18 g) as a colorless oil, which was used as such for next step. An analytical sample of tert-butyl methyl((3R,4R)-4-methylpiperidin-3-yl)carbamate was obtained by purification of this crude colorless oil by flash column chromatography. ¹H NMR (300 MHz, DMSO) δ 3.89 (s, 1H), 3.44 (q, J=7.0 Hz, 1H), 3.00-2.85 (m, 4H), 2.72 (dd, J=4.1, 12.2 Hz, 2H), 2.53 (d, J=15.0 Hz, 1H), 2.03 (m, 1H), 1.51 (m, 1H), 1.39 (s, 9H), 1.06 (t, J=7.0 Hz, 1H), 0.90 (d, J=7.2 Hz, 3H). MS (ES⁺): 229.2 (100%, M⁺¹).
c. To a solution containing mixture of tert-butyl methyl((3R,4R)-4-methylpiperidin-3-yl)carbamate (60) and (3R,4R)-tert-butyl 3-(tert-butoxycarbonyl(methyl)amino)-4-methylpiperidine-1-carboxylate (61) from the above step (11.4 g, 50 mmol) in methylene chloride (250 mL) cooled to 0° C. was added cyanoacetic acid (6.8 g, 80 mmol), EDCI (15.3 g, 80 mmol), triethylamine (14 mL, 100 mmol), HOBT (6.7 g, 50 mmol) and DMAP (0.6 g, 5 mmol). The reaction was allowed to warm to room temperature and stirred at room temperature overnight. The reaction mixture was washed with water (2×100 mL), dried over MgSO₄, and concentrated in vacuo. The residue obtained was purified by flash column chromatography (silica gel, 400 g, eluting with ethyl acetate in hexanes 0-70%) to furnish (3R,4R)-tert-butyl 3-(tert-butoxycarbonyl(methyl)amino)-4-methylpiperidine-1-carboxylate (61) (4.2 g, 28%) as an oil. ¹H NMR (300 MHz, DMSO) δ 3.91 (s, 1H), 3.53 (s, 2H), 3.39 (s, 1H), 3.2-3.05 (m, 1H), 2.77 (s, 3H), 2.03 (s, 1H), 1.49 (d, J=4.7 Hz, 2H), 1.39 (d, J=1.1 Hz, 18H), 0.91 (d, J=7.1 Hz, 3H). MS (ES⁺): 679.32 (100%, 2M^{+Na}); Analysis: Calcd for C₁₇H₃₂N₂O₄: C, 62.17; H, 9.82; N, 8.53. Found: C, 61.79; H, 9.72; N, 8.73. Further elution gave tert-butyl (3R,4R)-1-(2-cyanoacetyl)-4-methylpiperidin-3-yl(methyl)carbamate (62) (6.58 g, 45%) as a white solid; mp 118.3° C. ¹H NMR (300 MHz, DMSO) δ 4.16-4.01 (m, 2H), 4.00-3.85 (m, 1H), 3.71 (dd, J=6.9, 13.3 Hz, 1H), 3.66-3.38 (m, 2H), 3.25 (d, J=4.4 Hz, 1H), 2.75 (d, J=7.2 Hz, 3H), 2.10 (s, 1H), 1.69-1.44 (m, 2H), 1.40 (s, 9H), 0.93 (d, J=7.1 Hz, 3H); MS (ES⁺): 613.3 (100%, 2M^{+Na}); Analysis: Calcd for C₁₅H₂₅N₃O₃: C, 60.99; H, 8.53; N, 14.23. Found: C, 61.12; H, 8.60; N, 14.04.
d. To a solution of (3R,4R)-tert-butyl 3-(tert-butoxycarbonyl(methyl)amino)-4-methylpiperidine-1-carboxylate (61) (4.18 g, 12.73 mmol) in THF (32 mL) was added 4 M HCl in dioxane (64 mL, 254.6 mmol). The reaction was stirred at room temperature overnight. The solid obtained was collected by filtration, washed with ether and dried in vacuum to give (3R,4R)—N,4-Dimethylpiperidin-3-amine dihydrochloride (29) (2.49 g, 97%) as a white solid; mp 236.9° C. ¹H NMR (300 MHz, DMSO) δ 9.56 (s, 3H), 9.17 (s, 1H), 3.40 (d, J=13.5 Hz, 2H), 3.23 (s, 1H), 3.04 (s, 2H), 2.59 (s, 3H), 2.40 (s, 1H), 1.87 (s, 1H), 1.72 (s, 1H), 1.06 (d, J=7.1 Hz, 3H). MS (ES⁺): 129.3 (25%, M″); Analysis: Calcd for C₇H₁₈Cl₂N₂: C, 41.80; H, 9.02; N, 13.93; Cl, 35.25. Found: C, 41.60; H, 9.07; N, 13.45; Cl, 35.68.

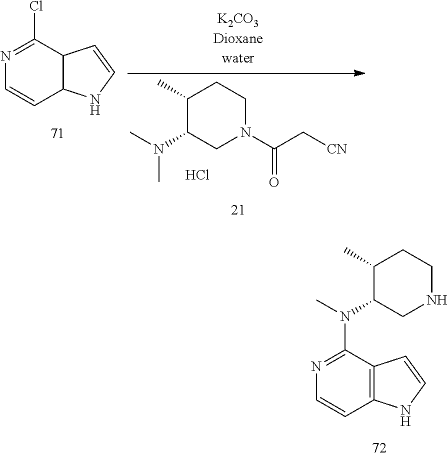

### Example 18

### N-Methyl-N-((3R,4R)-4-methylpiperidin-3-yl)-1H-pyrrolo[3,2-c]pyridin-4-amine (72)

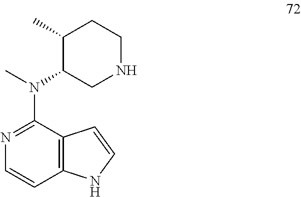

To a solution of 4-chloro-3a,7a-dihydro-1H-pyrrolo[3,2-c]pyridine (71) (WO2003009852, 0.1 g, 0.655 mmol) in dioxane (2 mL) was added 3-((3R,4R)-4-methyl-3-(methylamino)piperidin-1-yl)-3-oxopropanenitrile hydrochloride (21) (0.2 g, 0.86 mmol), potassium carbonate (0.475 g, 3.44 mmol), water (5 mL) and heated with stirring at 100° C. for 96 h. The reaction mixture was diluted with water (10 mL) and extracted with ethyl acetate (2×100 mL). The organic layers were combined washed with water (20 mL), brine (10 mL), dried and concentrated in vacuum. The residue obtained was purified by flash column chromatography (silica gel, 12 g, eluting with 0-50% CMA 80 in chloroform) to afford N-Methyl-N-((3R,4R)-4-methylpiperidin-3-yl)-1H-pyrrolo[3,2-c]pyridin-4-amine (72) (65 mg, 40.6%) as a beige solid; mp 126.9° C. ¹HNMR (300 MHz, DMSO) δ 11.29 (s, 1H), 7.68 (d, J=5.7, 1H), 7.32-7.14 (m, 1H), 6.81 (dd, J=0.8, 5.7, 1H), 6.57-6.41 (m, 1H), 3.66 (dt, J=6.9, 13.7, 2H), 3.44-3.36 (m, 2H), 3.34 (s, 3H), 2.59-2.52 (m, 1H), 2.00-1.86 (m, 1H), 1.58 (tdd, J=4.0, 9.2, 17.0, 3H), 0.96 (d, J=6.9, 3H); MS (ES⁺) 245.2 (100%: M⁺¹).

### Example 19

The following illustrate representative pharmaceutical dosage forms, containing a compound of formula I (‘Compound X’), for therapeutic or prophylactic use in humans.

| (i) Tablet 1 | mg/tablet |
|---|---|
| Compound X = | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |
| (ii) Tablet 2 | mg/tablet |
| Compound X = | 20.0 |
| Microcrystalline cellulose | 410.0 |
| Starch | 50.0 |
| Sodium starch glycolate | 15.0 |
| Magnesium stearate | 5.0 |
| | 500.0 |
| (iii) Capsule | mg/capsule |
| Compound X = | 10.0 |
| Colloidal silicon dioxide | 1.5 |
| Lactose | 465.5 |
| Pregelatinized starch | 120.0 |
| Magnesium stearate | 30 |
| | 600.0 |
| (iv) Injection 1 (1 mg/ml) | mg/ml |
| Compound X = (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |
| (v) Injection 2 (10 mg/ml) | mg/ml |
| Compound X = (free acid form) | 10.0 |
| Monobasic sodium phosphate | 0.3 |
| Dibasic sodium phosphate | 1.1 |
| Polyethylene glycol 400 | 200.0 |
| 01N Sodium hydroxide solution | q.s. |
| (pH adjustment to 7.0-7.5) | |
| Water for injection | q.s. ad 1 mL |
| (vi) Aerosol | mg/can |
| Compound X = | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A compound of formula I: 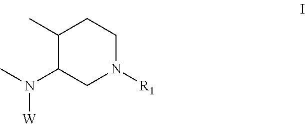
wherein
R₁ is H, alkyl, cycloalkyl, (cycloalkyl)alkyl, heterocycle, heteroaryl, aryl, wherein any alkyl, cycloalkyl, (cycloalkyl)alkyl, or heterocycle of R₁ may be optionally substituted with one or more Rₐ, and wherein any heteroaryl or aryl, of R₁ may be optionally substituted with one or more R_{c}; or R₁ is —C(R_{g})(R_{b})—C(Rₖ)(Rₘ)—CN;
each Rₐ group is independently selected from halogen, aryl, heteroaryl, heterocycle, R_{b}, OH, CN, OR_{b}, —O-aryl, —O-heterocycle, —O-heteroaryl, —OC(O)R_{b}, —OC(O)NHR_{b}, oxo, SH, SR_{b}, —S-aryl, —S-heteroaryl, —S(O)R_{b}, —S(O)aryl, —S(O)heteroaryl, —S(O)₂OH, —S(O)₂R_{b}, —S(O)₂aryl, —S(O)₂heteroaryl, —S(O)₂NH₂, —S(O)₂NHR_{b}, —S(O)₂NR_{b}R_{b}, —NH₂, —NHR_{b}, —NR_{b}R_{b}, —NHCOR_{b}, —NHCOaryl —NHCOheteroaryl, —NHCO₂R_{b}, —NHCONH₂, —NHCONHR_{b}, —NHS(O)₂R_{b}, —NHS(O)₂aryl, —NHS(O)₂NH₂, NO₂, ═NOR_{b}, CHO, —C(O)R_{b}, —C(O)OH, —C(O)OR_{b}, —C(O)NH₂, —C(O)NHR_{b}, —C(O)NR_{b}R_{b}, —C(O)heterocycle, —C(O)heteroaryl and —C(O)C(O)R_{b} and wherein any aryl, heteroaryl, or heterocycle of Rₐ may be optionally substituted with one or more R_{c} groups;
each R_{b} is independently lower alkyl or lower cycloalkyl wherein lower alkyl or lower cycloalkyl may be optionally substituted with one or more groups selected from halogen, CN, OH, —O-lower alkyl, —NH-lower alkyl, —C(O)NH-lower alkyl, —C(O)N(lower alkyl)₂, heterocycle and heteroaryl which heterocycle may be substituted with one or more lower alkyl;
each R_{c} is independently halogen, aryl, R_{d}, OH, CN, OR_{d}, —Oaryl, —OC(O)R_{d}, —OC(O)NHR_{d}, SH, SR_{d}, —S-aryl, —S-heteroaryl, —S(O)R_{d}, —S(O)aryl, —S(O)heteroaryl, —S(O)₂OH, —S(O)₂R_{d}, —S(O)₂aryl, —S(O)₂heteroaryl, —S(O)₂NHR_{d}, —S(O)₂NR_{d}R_{d}, —NH₂, —NHR_{d}, —NR_{d}R_{d}, —NHCOR_{d}, —NHCOaryl, —NHCOheteroaryl, —NHCO₂R_{d}, —NHCONH₂, —NHCONHR_{d}, —NHS(O)₂R_{d}, —NHS(O)₂aryl, —NHS(O)₂NH₂, NO₂, CHO, —C(O)R_{d}, —C(O)OH, —C(O)OR_{d}, —C(O)NH₂, —C(O)NHR_{d}, —C(O)NR_{d}R_{d}, —C(O)cyclic amino, —C(O)C(O)R_{d}, heterocycle or heteroaryl wherein any aryl may be optionally substituted with one or more Rₑ groups;
each R_{d} is independently lower alkyl or lower cycloalkyl wherein lower alkyl or lower cycloalkyl may be optionally substituted with one or more (e.g. 1, 2 or 3) groups selected from halogen, CN, OH, —O-lower alkyl, —NH-lower alkyl, —C(O)NH-lower alkyl, —C(O)N(lower alkyl)₂, heterocycle and heteroaryl which heterocycle may be substituted with one or more (e.g. 1, 2 or 3) lower alkyl;
each Rₑ is independently halogen, aryl, R_{f}, OH, CN, OR_{f}, —Oaryl, —OC(O)R_{f}, —OC(O)NHR_{f}, oxo, SH, SR_{f}, —S-aryl, —S-heteroaryl, —S(O)R_{f}, —S(O)aryl, —S(O)heteroaryl, —S(O)₂OH, —S(O)₂R_{f}, —S(O)₂aryl, —S(O)₂heteroaryl, —S(O)₂NHR_{f}, —S(O)₂NR_{f}R_{f}, —NH₂, —NHR_{f}, —NR_{f}R_{f}, —NHCOR_{f}, —NHCOaryl, —NHCOheteroaryl, —NHCO₂R_{f}, —NHCONH₂, —NHCONHR_{f}, —NHS(O)₂R_{f}, —NHS(O)₂aryl, —NHS(O)₂NH₂, NO₂, CHO, —C(O)R_{f}, —C(O)OH, —C(O)OR_{f}, —C(O)NH₂, —C(O)NHR_{f}, —C(O)NR_{f}R_{d}, —C(O)cyclic amino, —C(O)C(O)R_{d}, heterocycle or heteroaryl;
each R_{f} is independently lower alkyl or lower cycloalkyl wherein lower alkyl or lower cycloalkyl may be optionally substituted with one or more (e.g. 1, 2 or 3) groups selected from halogen, CN, OH, —O-lower alkyl, —NH-lower alkyl, —C(O)NH-lower alkyl, —C(O)N(lower alkyl)₂, heterocycle and heteroaryl which heterocycle may be substituted with one or more (e.g. 1, 2 or 3) lower alkyl;
R_{g} and Rₕ taken together are —CH₂—O—CH₂;
Rₖ and R_{T}, are each H, or taken together with the carbon to which they are attached form a C₃-C₆ spiro-carbocyclic ring; and
W is selected from: 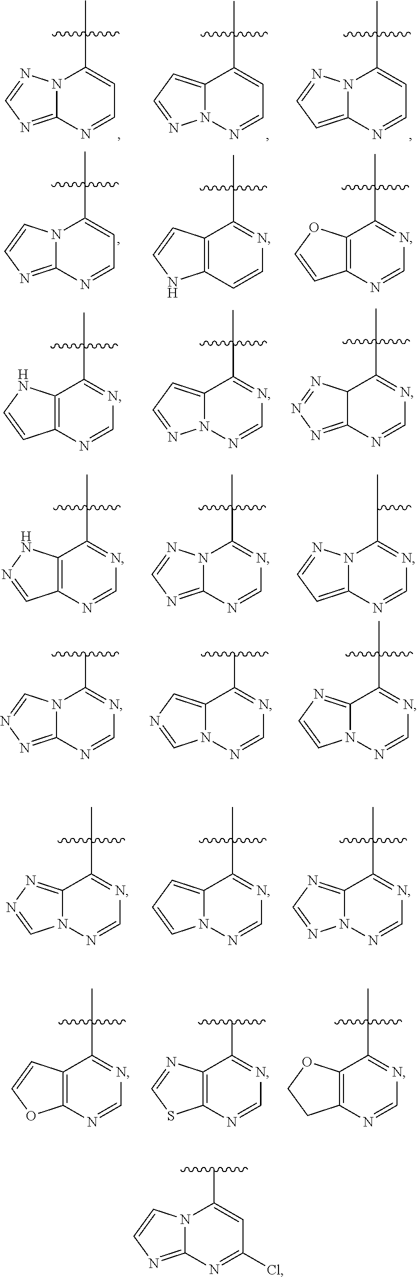 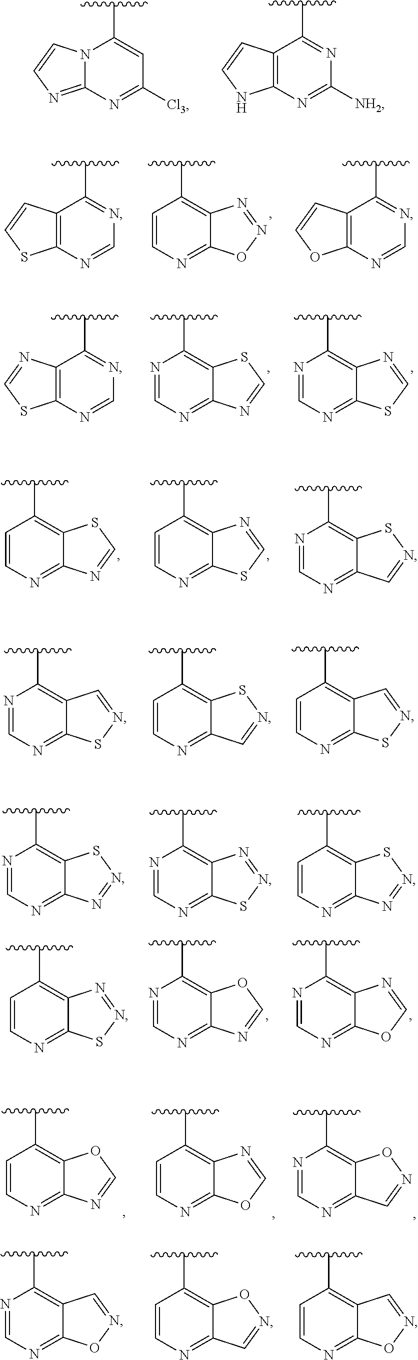 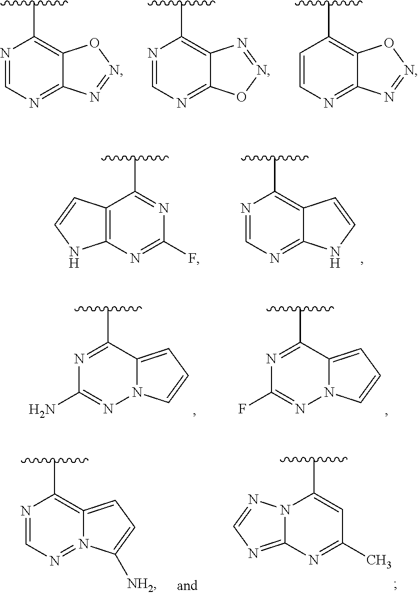
or a salt thereof;
provided the compound of formula I is not:

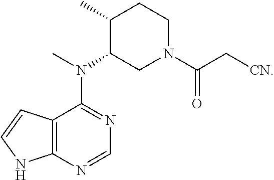

2. The compound of claim 1 which is a compound of formula Ia: 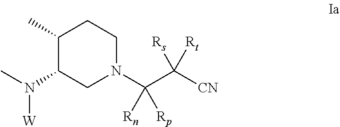
wherein:
Rₙ and Rₚ taken together are oxo (═O) or —CH₂—O—CH₂;
Rₛ and Rₜ are each H, or taken together with the carbon to which they are attached form a C₃-C₆ spiro-carbocyclic ring; and
W has any of the values defined in claim 1;
or a salt thereof.

3. The compound of claim 2 wherein the compound of formula Ia is a compound of formula Ib:

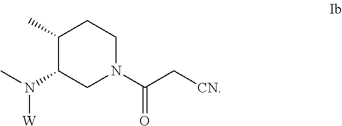

4. The compound of claim 3 wherein W is selected from:

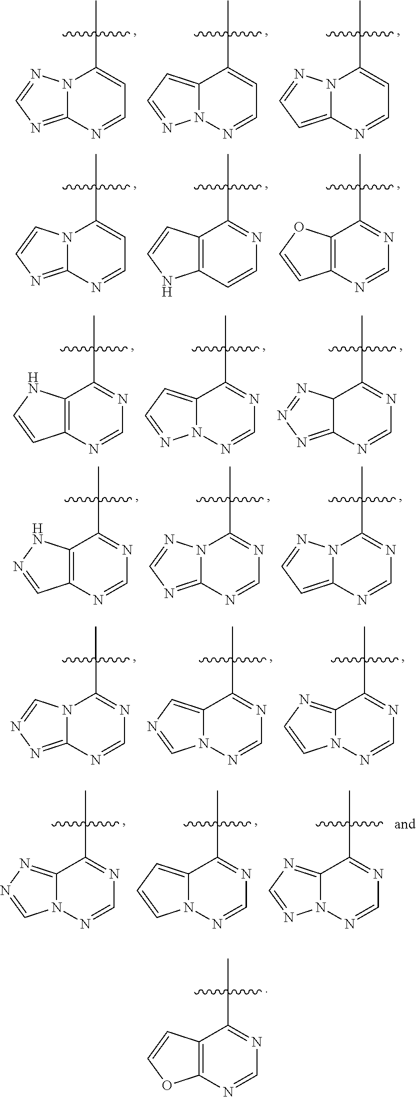

5. The compound of claim 2 wherein Rₙ and Rₚ taken together are oxo (═O).

6. The compound of claim 2 wherein Rₙ and Rₚ taken together are —CH₂—O—CH₂—.

7. The compound of claim 2 wherein Rₛ and Rₜ are each H.

8. The compound of claim 2 wherein Rₛ and Rₜ taken together with the carbon to which they are attached form a C₃-C₆ spiro-carbocyclic ring.

9. The compound of claim 2 wherein Rₛ and Rₜ taken together with the carbon to which they are attached form a C₃ spiro-carbocyclic ring.

10. The compound of claim 1 wherein W is selected from:

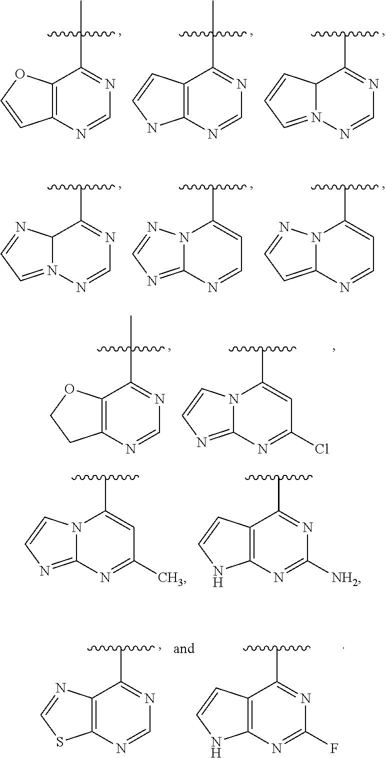

11. The compound of claim 1 wherein the compound of formula I is a compound having the structure

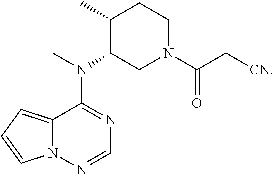

12. The compound of claim 1 wherein the compound of formula I is a compound having the structure

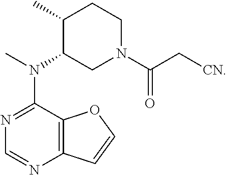

13. The compound of claim 1 which is a compound of formula, 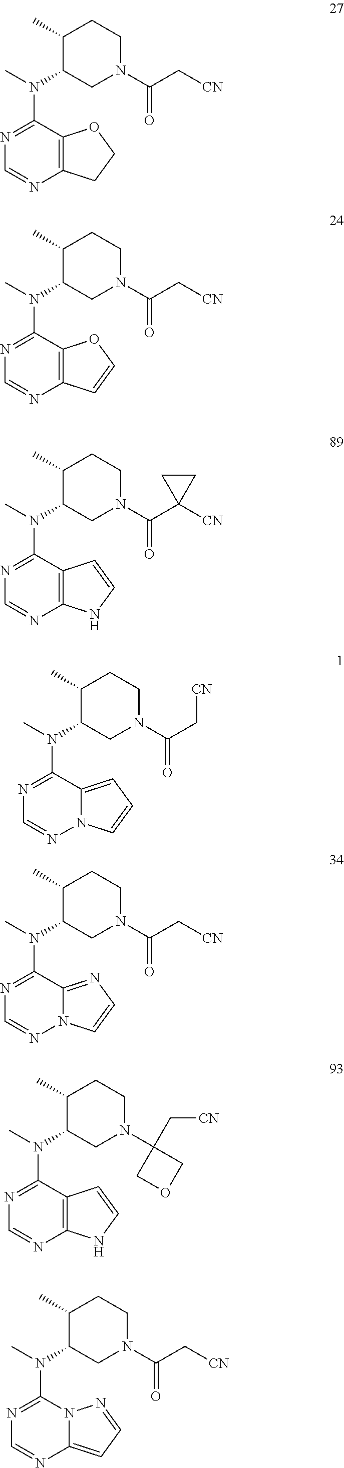 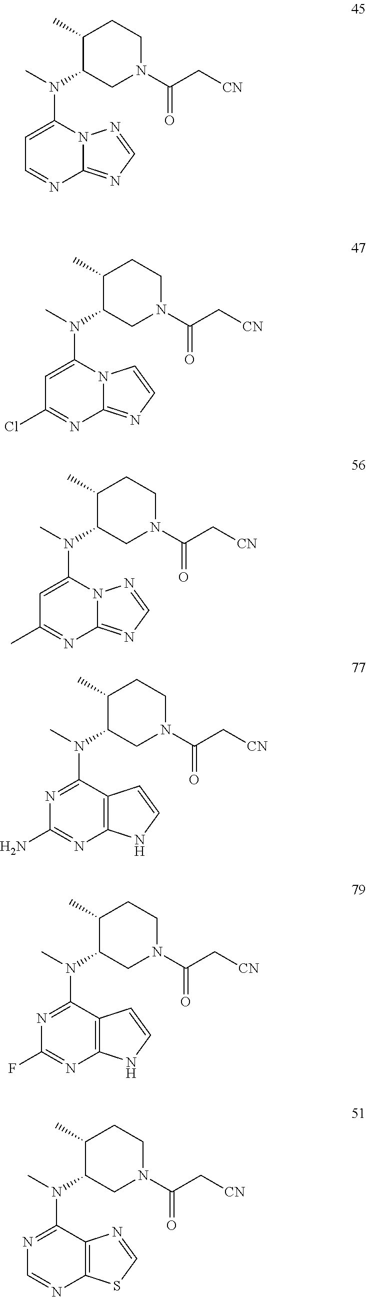 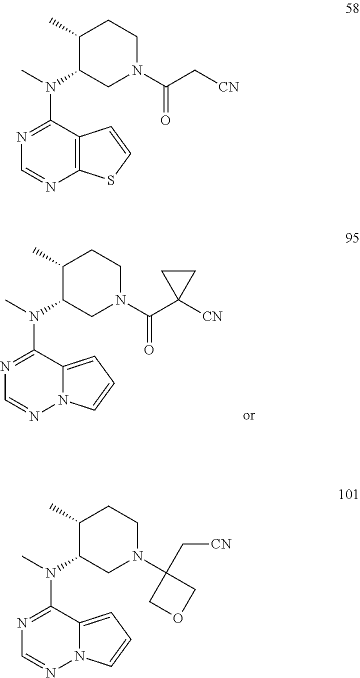
or a salt thereof.

14. The compound of claim 1 which is a compound of formula, 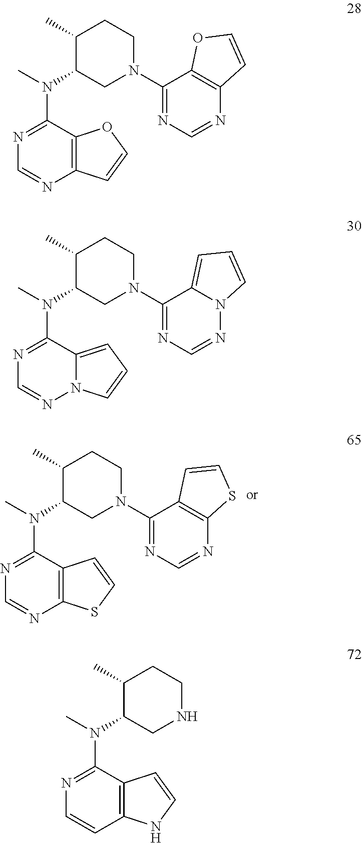
or a salt thereof.

15. The compound of claim 1 which is a compound of formula: 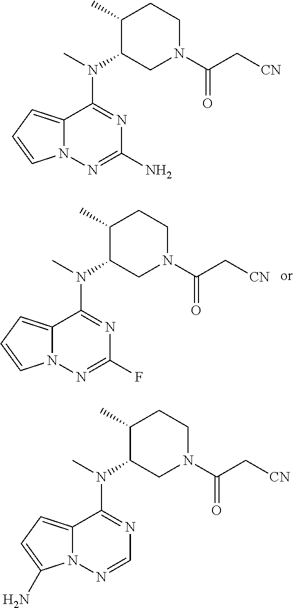
or a salt thereof.

16. A pharmaceutical composition comprising a compound of formula I as described in claim 1, or a pharmaceutically acceptable salt thereof, in combination with a pharmaceutically acceptable diluent or carrier.

17. A method for treating a disease or condition associated with pathologic Jak activation in a mammal, comprising administering a compound of formula I as described in claim 1, or a pharmaceutically acceptable salt thereof, to the mammal.

18-20. (canceled)

21. The method of claim 17, wherein the disease or condition associated with pathologic Jak activation is cancer.

22. The method of claim 17, wherein the disease or condition associated with pathologic Jak activation is a hematologic or other malignancy.

23. A method for suppressing an immune response in a mammal, comprising administering a compound of formula I as described in claim 1 or a pharmaceutically acceptable salt thereof, to the mammal.

24-25. (canceled)

26. A method for preparing a compound of formula I or a salt thereof as described in claim 1 comprising: 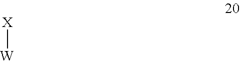
a. reacting a corresponding compound of formula 20:
wherein X is a suitable leaving group with a corresponding compound of formula 102: 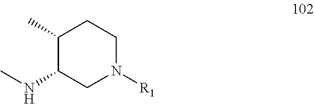
to provide the compound of formula I or the salt thereof; or
b. reacting a corresponding compound of formula 104: 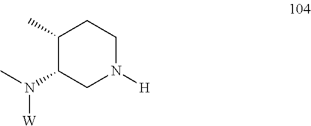
with a corresponding compound of formula R₁—X, wherein X is a suitable leaving group, to provide the compound of formula I.
